(19) United States
(12) Patent Application Publication
(10) Pub. No.: US 2023/0277121 A1
(43) Pub. Date: Sep. 7, 2023

(54) DISEASE BURDEN INDICATION

(71) Applicant: INSPIRE MEDICAL SYSTEMS, INC., Golden Valle, MN (US)

(72) Inventors: Christopher Thorp, Minneapolis, MN (US); Nicholas Nesbitt, Minneapolis, MN (US); John Rondoni, Plymouth, MN (US); David Dieken, Minneapolis, MN (US); Kevin Verzal, Lino Lakes, MN (US); Heather Orser, Farmington, MN (US)

(73) Assignee: INSPIRE MEDICAL SYSTEMS, INC., Golden Valley, MN (US)

(21) Appl. No.: 18/017,797

(22) PCT Filed: Jul. 21, 2021
(86) PCT No.: PCT/US2021/042601

§ 371 Date: Jan. 24, 2023

Related U.S. Application Data
(60) Provisional application No. 63/056,241, filed on Jul. 24, 2020.

(51) Int. Cl. A61B 5/00; A61B 5/0205; A61N 1/36

(52) U.S. Cl. CPC A61B 5/4818; A61B 5/0205; A61B 5/746; A61N 1/36078; A61N 1/36139; A61B 2562/0219

(57) ABSTRACT

Methods and/or devices to identify disease burden indication are disclosed. One type of disease comprises sleep disordered breathing and/or related parameters, which may be sensed via implantable sensors such as an acceleration sensor.

### BACKGROUND

A significant portion of the population suffers from various forms of diseases, the burden of which is assessed and/or for which various therapies are applicable. One example disease comprises sleep disordered breathing (SDB). In some patients, external breathing therapy devices and/or mere surgical interventions may fail to treat the sleep disordered breathing behavior.

### BRIEF DESCRIPTION OF THE DRAWINGS

FIG. 1A is a flow diagram schematically representing an example method of identifying disease burden indication.

FIG. 1B is a diagram including a front view schematically representing a patient’s body to which example methods and/or example devices may be applied.

FIG. 2A is a flow diagram schematically representing an example method of identifying disease burden indication.

FIG. 2B is a diagram including a front view of a patient’s body schematically representing an example method and/or example device for identifying disease burden indication.

FIG. 2C is a diagram schematically representing example deployments of an accelerometer relative to patient body portions.

FIG. 3A is a diagram including a side view, schematically representing an example method and/or example device for detecting respiration via an acceleration sensor implanted at a chest wall.

FIG. 3B is a diagram, including a side view, schematically representing an example method and/or example device for detecting respiration via an acceleration sensor at a chest wall.

FIG. 3C is a diagram including a graph schematically representing an example filtered, sensed acceleration signal.

FIG. 4 is a diagram schematically representing an example method of identifying a disease burden indicator.

FIG. 5 is a diagram schematically representing an example method of applying therapy in association with an identified disease burden indicator.

FIG. 6 is a diagram schematically representing an example method of implementing the identification via a first control portion.

FIG. 7 is a diagram schematically representing an example method of implementing the identification via a second control portion.

FIGS. 8 and 9 are each a diagram schematically representing an example method of constructing and training a data model, respectively.

FIG. 10A is a block diagram schematically representing example types of a data model.

FIG. 10B is a diagram schematically representing an example method of implementing construction of a data model via an external resource.

FIG. 11A is a block diagram schematically representing an example method of constructing a data model.

FIG. 11B is a block diagram schematically representing an example method of determining a disease burden indicator.

FIG. 11C is a flow diagram schematically representing an example method of identifying a disease burden indicator.

FIG. 11D is a block diagram schematically representing an example criteria for identifying a disease burden indicator.

FIG. 12A is a flow diagram schematically representing an example method and/or example device for constructing a data model according to known inputs and known outputs.

FIG. 12B is a flow diagram schematically representing an example method and/or example device determining a current disease burden indicator via a constructed data model.

FIG. 13A is a block diagram schematically representing example measurable physiologic parameters.

FIG. 13B is a diagram schematically representing an example method of constructing a data model.

FIG. 14 is a block diagram schematically representing example known input sources for constructing a data model.

FIG. 15 is a block diagram schematically representing example motion input sources.

FIG. 16A is a block diagram schematically representing example arousal-related input sources.

FIG. 16B is a block diagram schematically representing example known input sources relating to at least breathing.

FIG. 17A is a flow diagram schematically representing an example method of identifying sleep disordered breathing.

FIG. 17B is a block diagram schematically representing an example method of constructing a data model regarding identifying blood oxygen desaturation.

FIG. 17C is a block diagram schematically representing an example method of identifying blood oxygen desaturation via a constructed data model.

FIG. 18 is a flow diagram schematically representing an example method of identifying sleep disordered breathing based on at least blood oxygen desaturation.

FIG. 19 is a block diagram schematically representing an example criteria for identifying sleep disordered breathing.

FIG. 20 is a block diagram schematically representing an example method and/or example device for constructing a data model to identify blood oxygen desaturation.

FIG. 21 is a block diagram schematically representing an example method and/or example device for identifying blood oxygen desaturation based on a constructed data model.

FIG. 22 is a block diagram schematically representing an example method and/or example device for constructing a data model to identify blood oxygen desaturation based on at least some externally sensed known inputs.

FIG. 23 is a diagram schematically representing an example method and/or example device for identifying blood oxygen desaturation based on a constructed data model.

FIG. 24 is a flow diagram schematically representing an example method of identifying sleep disordered breathing via at least identifying surrogates for externally measured blood oxygen desaturation.

FIG. 25A is a flow diagram schematically representing an example criteria for identifying a disease burden indicator in relation to a respiratory signal.

FIG. 25B is a block diagram schematically representing an example criteria for identifying a disease burden indicator.

FIG. 25C is a block diagram schematically representing an example method and/or example device for constructing a data model in association with a sensed respiratory signal.

FIG. 26 is a block diagram schematically representing an example method and/or example device for determining disease burden indication according to a constructed data model.

FIG. 27 is a block diagram schematically representing an example method and/or example device for identifying disease burden indication according to example respiratory-related parameters.

FIG. 28 is a flow diagram schematically representing an example method and/or example device for identifying a disease burden indicator in relation to a duration of a respiratory cycle.

FIG. 29 is a block diagram schematically representing an example method and/or example device for constructing a data model to identify a disease burden indicator based on internally sensed known inputs and at least some externally sensed known inputs.

FIG. 30 is a diagram schematically representing an example method and/or example device for identifying a disease burden indicator based on a constructed data model.

FIG. 31 is a flow diagram schematically representing an example method of identifying a disease burden indicator via identifying surrogates for externally measured respiration information.

FIG. 32A is a flow diagram schematically representing an example method of identifying an arousal.

FIG. 32B is a block diagram schematically representing an example method and/or example device for constructing a data model, in association with sensed physiologic information, to identify an arousal.

FIG. 33A is a block diagram schematically representing an example method and/or example device for constructing a data model to identify an arousal based on internally sensed known inputs and at least some externally sensed known inputs.

FIG. 33B is a block diagram schematically representing at least some example known inputs related to arousals.

FIG. 34 is a diagram schematically representing an example method and/or example device for identifying an arousal according to a constructed data model.

FIGS. 35 and 36 are each a block diagram schematically representing an example method and/or example device for differentiating different types of sleep apnea.

FIG. 37 is a block diagram schematically representing example measurement types regarding disease burden indication.

FIG. 38 is a diagram schematically representing an example method of gathering sensed physiologic information.

FIG. 39 is a flow diagram schematically representing an example method for updating therapy settings and/or sensor settings via at least one external resource.

FIG. 40 is a block diagram schematically representing an example method of performing therapy via updated therapy settings and sensor settings.

FIG. 41 is a flow diagram schematically representing an example method for updating therapy settings and/or sensor settings via updating construction of a data model.

FIG. 42 is a flow diagram schematically representing an example method for importing an updated constructed data model, including updated therapy settings and/or sensor settings, into an implantable medical device.

FIG. 43 is a flow diagram schematically representing an example method for performing therapy via updated therapy settings and/or updated sensor settings.

FIG. 44A is a flow diagram schematically representing an example method for updating construction of a data model using an externally measurable physiologic parameter and importing the updated data model into an implantable medical device.

FIG. 44B is a block diagram schematically representing an example method for updating therapy settings and/or sensor settings via an externally measurable physiologic parameter.

FIG. 44C is a block diagram schematically representing an example method of importing, into an implantable medical device, updated therapy and sensor settings.

FIG. 44D is a block diagram schematically representing an example method of performing, within an implantable medical device, updating therapy and sensor settings.

FIG. 44E is a block diagram schematically representing an example method of updating therapy settings and sensor settings at a location external to the patient’s body.

FIG. 44F is a block diagram schematically representing an example method of updating therapy settings and sensor settings via updating construction of a data model.

FIGS. 44G and 44H are each a block diagram schematically representing an example method of updating construction of a data model.

FIGS. 45, 46, 47, and 48 are each a block diagram schematically representing an example method for reducing disease burden indication via adjusting therapy and/or sensor settings.

FIG. 49 is a block diagram schematically representing an example method for performing a sweep of therapy settings and/or sensor settings over a treatment period.

FIG. 50 is a diagram including a front view of a patient’s body and schematically representing an example method and/or example device for treating disease burden, with an implanted medical device, sensor, and stimulation lead.

FIG. 51 is a diagram including a front view of a patient’s body and schematically representing an example method and/or example device for treating disease burden, with an implanted microstimulator and sensor.

FIG. 52A is a block diagram schematically representing an example care engine.

FIGS. 52B and 52C are each a block diagram schematically representing an example control portion.

FIG. 52D is a block diagram schematically representing an example user interface.

FIG. 52E is a diagram schematically representing an example arrangement of communication between an implantable medical device and various example external devices.

FIG. 53A is a diagram schematically representing an example method and/or example device for constructing a data model for identifying disease burden indication and/or an externally measurable physiologic parameter.

FIG. 53B is a block diagram schematically representing an example class arrangement.

FIG. 53C is a block diagram schematically representing an example trend parameter.

FIG. 54 is a block diagram schematically representing example relationships between measurable physiologic parameters, disease burden indicators, and therapy modalities.

FIGS. 55A, 55B, and 55C are each a flow diagram schematically representing an example method and/or example device of identifying, via a constructed data model, a disease burden indicator and/or physiologic parameter.

FIGS. 56A and 56B are each a diagram, including a side view, schematically representing an example method and/or example device for detecting respiration via an acceleration sensor.

FIG. 56C is a diagram schematically representing example acceleration sensing elements.

FIG. 57A is a diagram including a side view schematically representing an example method and/or example device for detecting respiration with a patient relative to an angled support.

FIGS. 57B and 57C are each a diagram schematically representing an example method and/or example device including a sensing element extending at a particular angle relative to a gravity vector.

FIG. 58 is a diagram including a side view schematically representing an example method and/or example device for detecting respiration with a patient relative to an upright support.

FIG. 59 is a diagram including a front view schematically representing an example method and/or example device in which different sensing elements of an acceleration sensor are oriented relative to a patient’s body.

FIG. 60 is a diagram including a side view schematically representing an example method and/or example device in which different sensing elements of an acceleration sensor are oriented relative to a patient’s body.

FIG. 61A is a diagram including a front view schematically representing an example method and/or example device including an implantable medical device comprising an acceleration sensor.

FIG. 61B is a diagram schematically representing an example method of arranging an acceleration sensor.

FIG. 61C is a diagram schematically representing an example method of identifying a sensing element in relation to a reference angular orientation.

FIG. 61D is a diagram schematically representing an example method of determining a reference angular orientation.

FIG. 61E is a diagram schematically representing an example method of implementing sensing.

FIG. 61F is a diagram schematically representing an example method of determining respiration information via an identified sensing element.

FIG. 61G is a diagram schematically representing an example method of sensing within a range of angular orientations.

FIG. 61H is a diagram schematically representing an example method of identifying a sensing element exhibiting a reference angular orientation.

FIG. 61I is a diagram schematically representing an example method of determining respiration information using an identified sensing element in relation to a greatest range of angular orientations.

FIG. 61J is a diagram schematically representing an example method of identifying a sensing element in relation to a greatest range of values of an AC signal component.

FIG. 61K is a diagram schematically representing an example method of determining respiration information using an identified sensing element in relation to a greatest range of values of an AC signal component.

FIG. 61L is a diagram schematically representing an example method of sensing an AC signal component during breathing.

FIG. 62 is a diagram including a side view of a patient’s chest and which schematically represents an example method of determining respiration information based on sensing rotational movement of the chest during breathing.

FIG. 63 is a diagram including a side view schematically representing different angular orientations upon rotation of an acceleration sensor relative to a gravity vector.

FIGS. 64, 65, 66A are each a diagram including a side view schematically representing an implantable medical device including two spaced apart, acceleration sensors and arranged in different configurations relative to each other.

FIG. 66B is a diagram schematically representing an offset of angular orientation of the respective sensing elements of two spaced apart accelerometers.

FIGS. 67 and 68 are each a diagram including a side view schematically representing an implantable medical device including two spaced apart, acceleration sensors and arranged in different configurations relative to each other.

FIG. 69A is diagram schematically representing an example method and/or example device for detecting noise using two spaced apart accelerometers with one accelerometer in an implantable medical device to sense respiration and the other accelerometer spaced apart from the respiration sensing region.

FIG. 69B is a diagram including a top view schematically representing an example implantable pulse generator including a lead comprising an accelerometer.

FIGS. 70 and 71 are each a diagram including a side view of a patient’s chest and which schematically represents an example method of determining respiration information based on sensing rotational movement of the chest, via a sensor mounted on a side of the chest.

FIG. 72 is a diagram including a front view schematically representing an example method of sensing respiration via a sensor on a side portion of a patient’s chest.

FIG. 73 is a diagram schematically representing an example method of determining respiration information via sensing rotation of a sensing element in relation to rotation of a side of a patient’s chest.

FIG. 74 is a diagram schematically representing an example method and/or example device for determining respiration information via a sensed acceleration signal.

FIG. 75A is a block diagram schematically representing an example confidence factor portion.

FIG. 75B is a block diagram schematically representing an example feature extraction portion.

FIG. 75C is a block diagram schematically representing an example inspiratory phase prediction function.

FIG. 75D is a block diagram schematically representing an example noise model parameter.

FIG. 75E is a block diagram schematically representing an example care engine.

FIG. 76A is a block diagram schematically representing an example method of determining respiration information in relation to sensing rotation of a respiratory body portion.

FIG. 76B is a block diagram schematically representing an example method of determining respiration information in relation to sensing rotation of a chest wall of patient.

FIG. 77 is a block diagram schematically representing an example method of sensing rotation in relation to an earth gravity vector.

FIGS. 78 and 79 each are a block diagram schematically representing an example method of sensing rotational movement in relation to particular orthogonal axes.

FIG. 80 is a block diagram schematically representing an example method of determining respiration information for a first body position.

FIG. 81 is a block diagram schematically representing an example method of determining respiration information without separately identifying translation motion.

FIG. 82 is a block diagram schematically representing an example method of determining respiration information without implant orientation calibration.

FIG. 83 is a block diagram schematically representing an example method of determining respiration information in relation to pitch, yaw, and roll.

FIG. 84 is a block diagram schematically representing an example method of selecting an implant location.

FIG. 85 is a block diagram schematically representing an example method of determining respiration information while excluding information regarding cardiac, muscle, and/or noise.

FIG. 86 is a block diagram schematically representing an example method of determining respiration information via subtracting noise.

FIG. 87 is a block diagram schematically representing an example method of determining respiration information via measuring inclination relative to an earth gravity vector.

FIG. 88 is a block diagram schematically representing an example method of determining respiration information without determining body position.

FIG. 89 is a block diagram schematically representing an example method of determining respiration information while the patient is in different body positions.

FIGS. 90 and 91 each are a block diagram schematically representing an example method of determining respiratory morphology.

FIGS. 92 and 93 each are a block diagram schematically representing an example method of determining respiration information in relation to a confidence information.

FIG. 94 each are a block diagram schematically representing an example method of extracting respiratory phase information in relation to thresholds.

FIG. 95 is a block diagram schematically representing an example method of determining respiration information in relation to body position.

FIG. 96 is a block diagram schematically representing an example method of determining respiration information based on sensing rotational movement of an abdomen.

FIG. 97 is a diagram, including a side view, schematically representing an example method and/or example device for detecting respiration via an acceleration sensor at an abdominal wall.

FIG. 98 is a diagram, including a front view, schematically representing an example method and/or example device for detecting respiration via multiple sensing elements of an acceleration sensor at an abdominal wall.

FIG. 99 is a diagram, including a front view, schematically representing an example method and/or example device for treating sleep disordered breathing via a medical device implanted at an abdomen to stimulate a phrenic nerve in the abdomen and including an acceleration sensor.

FIG. 100 is a diagram, including a front view, schematically representing an example method and/or example device for treating sleep disordered breathing via a medical device implanted in a pectoral region to stimulate a phrenic nerve in the abdomen and an acceleration sensor implanted in the abdomen.

FIG. 101 is a diagram, including a front view, schematically representing an example method and/or example device for treating sleep disordered breathing via a medical device, including an acceleration sensor, implanted in a pectoral region to stimulate a phrenic nerve in the head-and-neck region.

FIG. 102 is a diagram, including a front view, schematically representing an example method and/or example device for treating sleep disordered breathing via a microstimulator implanted in a head-and-neck region to stimulate a phrenic nerve in the head-and-neck region.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to using a sensor(s) to identify and/or track disease burden, which may be expressed as a disease burden indicator in some examples. In some examples, the disease burden indicator may comprise and/or be expressed as a measurable physiologic parameter(s). In some examples, the sensors may be implantable and/or may be externally located from the patient. In some such examples, one example implantable sensor may comprise an implantable accelerometer.

In some examples, the disease burden indicator may comprise a sleep disordered breathing (SDB) indicator and/or related parameters, etc. In some examples, the implantable acceleration sensor may sense physiologic information, including but not limited to, respiratory information to identify the sleep disordered breathing. In some such examples, the sensed physiologic information may comprise respiratory motion, such as but not limited to, rotational chest motion of the patient.

In some instances, identifying a disease burden indicator may comprise using an implantable sensor to estimate physiologic information which typically is measured externally of the patient. In the example of sleep disordered breathing, such physiologic information may comprise parameters such as respiratory airflow, blood oxygen desaturation, and/or related parameters associated with identifying sleep disordered breathing (SDB).

For instance, some example methods and/or devices may use various types of information internally sensed via the implantable sensor(s) (e.g. acceleration sensor) to approximate and/or estimate the measurable physiologic parameters used to identify disease burden indication. To implement this arrangement, example methods and/or devices may determine which internally sensed physiologic information provides the best estimation of the typically externally sensed information. This determination may comprise correlating (or otherwise comparing) the internally sensed physiologic information with the externally sensed physiologic information.

In this way, the use of external sensors may be avoided (in some examples) and the internal sensors may be the sole means of obtaining the physiologic information to identify the disease burden indication, such as but not limited to sleep disordered breathing (SDB). In some examples in which the internally sensed physiologic information is obtained via an implantable accelerometer, the accelerometer may form part of an implantable medical device (IMD) such that little or no tunneling, or no separate invasive implantation procedures are used to implant sensing elements (e.g. pressure sensors, impedance sensors, and the like). In some such examples, the implantable medical device may comprise an implantable pulse generator. Via such arrangements, the implantation of medical device or system may be simplified, thereby reducing cost, time, complexity, etc.

In some examples, example methods and/or devices may utilize data model techniques to determine which internally sensed physiologic information (e.g. inputs) provides the best estimation of the typically externally sensed physiologic information. In some such examples, the data model may comprise a machine learning model (MLM). The data model may be trained according to known inputs and known output(s) prior to applying the data model to identify disease burden indication using current inputs which are internally sensed via implantable sensing elements (e.g. acceleration sensor).

It will be further understood that in some instances, a data model may be used to identify just some of the internally sensed inputs and/or just some of the ways in which the internally sensed inputs may be used to identify sleep disordered breathing, such that non-data-model techniques may be used with (or without) the data model techniques to determine the desired internally sensed inputs.

Accordingly, it will be further understood that aspects of the various example methods involving non-data models and those involving data models may be selectively mixed and matched with each other as desired to achieve the desired and/or optimal manner of identifying disease burden indication via internally sensed physiologic information.

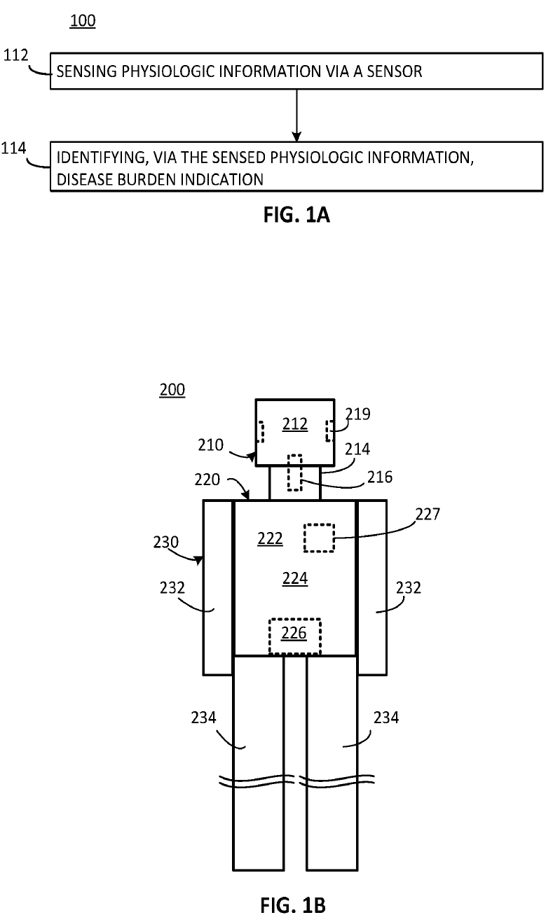

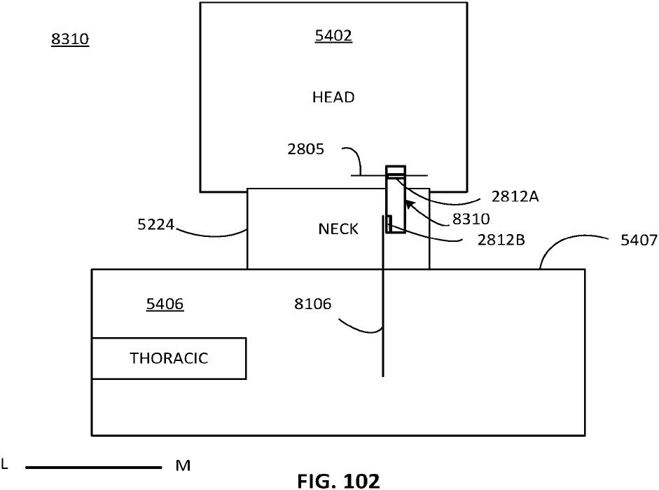

At least these examples, and additional examples, are described in association with at least FIGS. 1A-102.

FIG. 1A is a flow diagram schematically representing an example method 100. As shown at 112 in FIG. 1A, in some examples method 100 comprises sensing physiologic information via a sensor, and as shown at 114, method 100 may further comprise identifying, via the sensed physiologic information, disease burden indication (e.g. a disease burden indicator). In some examples, the disease burden indicator may comprise a sleep disordered breathing (SDB) indicator. In some examples, the sleep disordered breathing (SDB) may comprise an apnea and/or a hypopnea. In some examples, the sleep disordered breathing (SDB) may comprise apneas, which may be obstructive and/or central, as well as hypopneas in some instances. Additional aspects of sleep disordered breathing are further described throughout the present disclosure.

In some examples, the disease burden indicator may comprise indicia of diseases other than sleep disordered breathing, such as but not limited to those described in association with at least FIGS. 4-12B and 53A-55C.

Moreover, it will be apparent from the examples throughout the present disclosure that, in some example methods and/or devices, non-physiologic information may be sensed and/or that sensing of the physiologic information (or non-physiologic information) may be performed via an acceleration sensor(s) and/or sensors other than an acceleration sensor.

FIG. 1B is block diagram schematically representing a patient’s body 200, including example target portions 210-234 at which at least some example sensing elements may be employed to implement at least some examples of the present disclosure.

As shown in FIG. 1B, patient’s body 200 comprises a head-and-neck portion 210, including head 212 and neck 214. Head 212 comprises cranial tissue, nerves, etc., which may include auditory portions 219 (e.g., hearing organs, nerves) and upper airway 216 (e.g., nerves, muscles, tissues), etc. As further shown in FIG. 1B, the patient’s body 200 comprises a torso 220, which comprises various organs, muscles, nerves, other tissues, such as but not limited to those in pectoral region 222 (e.g., cardiac 227), abdomen 224, and/or pelvic region 226 (e.g., urinary/bladder, anal, reproductive, etc.). As further shown in FIG. 1B, the patient’s body 200 comprises limbs 230, such as arms 232 and legs 234.

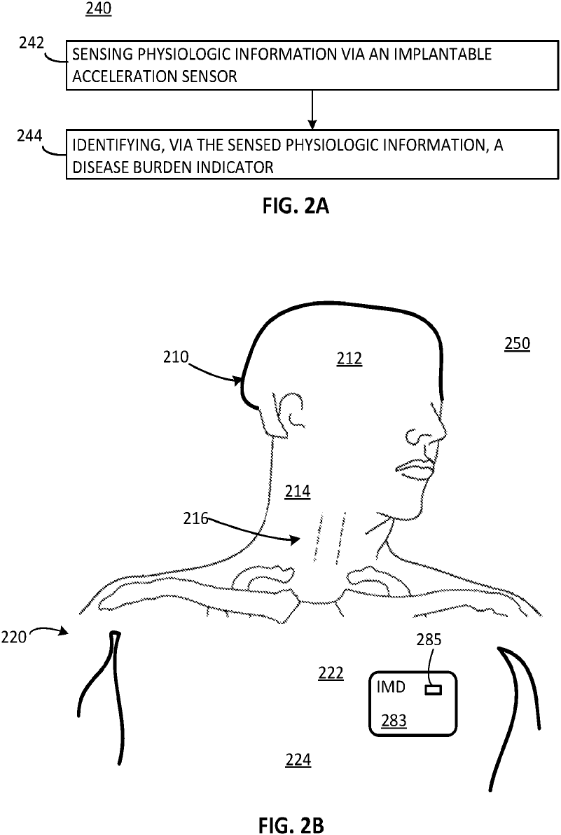

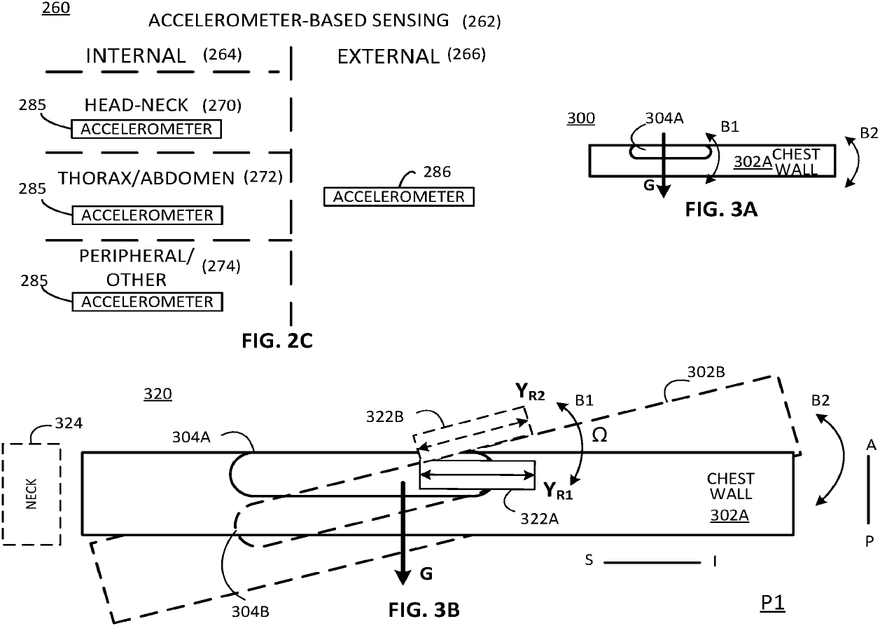

It will be understood that various sensing elements (and/or stimulation elements) as described throughout the various examples of the present disclosure may be deployed within the various regions of the patient’s body 200 in order to sense and/or otherwise diagnose, monitor, treat various physiologic conditions such as, but not limited to those examples described below in association with FIG. 2A-102.

FIG. 2A is a flow diagram schematically representing an example method 240. As shown at 242 in FIG. 2A, in some examples method 240 comprises sensing physiologic information via an implantable acceleration sensor, and as shown at 244, method 240 may further comprise identifying, via the sensed physiologic information, a disease burden indicator. In some examples, the disease burden indicator may comprise a sleep disordered breathing (SDB) indicator. In some examples, the sleep disordered breathing (SDB) may comprise an apnea and/or a hypopnea. As noted elsewhere below, in some particular contexts, other behaviors may sometimes be considered sleep disordered breathing (SDB). In some examples, the disease burden indicator may comprise indicia of diseases other than sleep disordered breathing, such as but not limited to those described in association with at least FIGS. 4-12B and 53A-55C.

FIG. 2B is diagram 250 including a front view schematically representing deployment within a patient’s body of an example implantable medical device (IMD) 283, which includes an acceleration sensor 285. In some examples, at least some of the reference numerals described in association with FIG. 1B to identify various portions of the patient’s body are also applied to identify similar portions of the patient’s body in FIG. 2B. As shown in FIG. 2B, in some examples the IMD 283 (and therefore acceleration sensor 285) may be chronically implanted in a pectoral region 222 of a patient’s body 200. The acceleration sensor 285 may sense various physiologic phenomenon from this implanted position, which includes at least respiration information in some examples. Additional physiologic information sensed via acceleration sensor 285 is further described below.

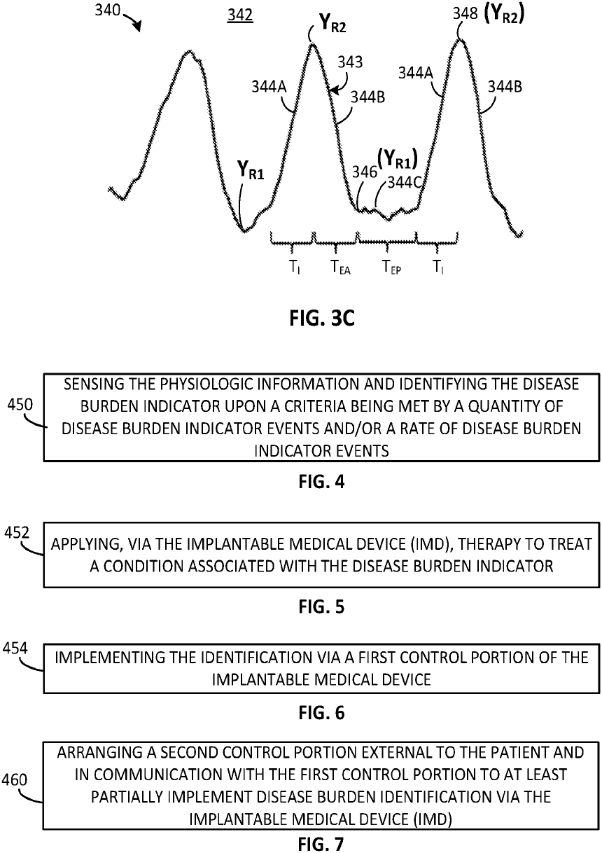

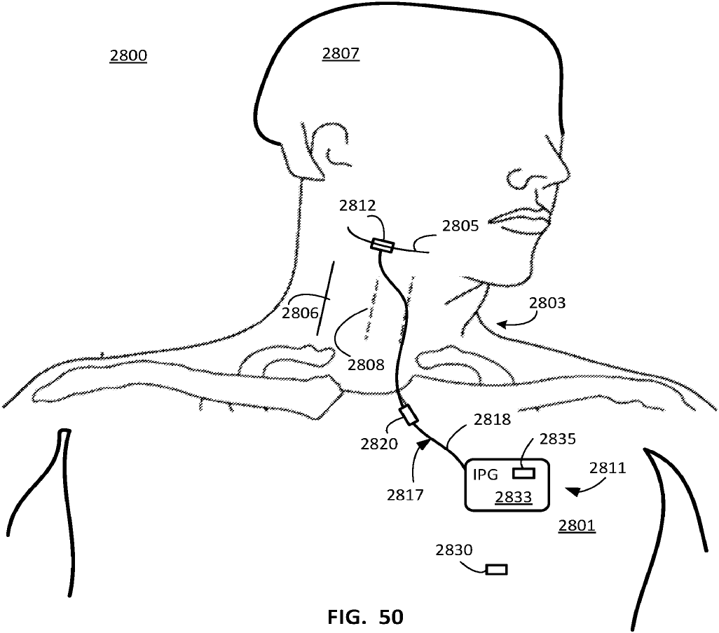

In some examples, the respiration information sensed via acceleration sensor 285 may comprise a respiratory waveform from which, at least sleep disordered breathing (SDB) and/or other disease burden indicators may be identified. Sensing this respiration information is further described below in association with at least FIGS. 3A-3C and FIGS. 56A-102. In some examples, the IMD 283 may comprise an implantable pulse generator (IPG), such as for managing sensing and/or SDB stimulation therapy, as later described in association with at least FIGS. 50-52D.

FIG. 2C is a diagram 260 schematically representing example acceleration-sensing arrangements 262 in which an acceleration sensor (e.g. accelerometer) 285 may be deployed relative to a patient’s body. As shown in FIG. 2C, in some examples an accelerometer 285 may be implanted internally (264) such as in a head-and-neck region 270, a thorax/abdomen region 272, or a peripheral/other region 274. In some examples, more than one accelerometer 285 may be implanted in a single region and/or in different multiple regions in the patient’s body. As further shown in FIG. 2C, in some examples an accelerometer 286 (like 285) may be deployed external (266) to a patient’s body. In some examples, at least one accelerometer 285 may be implanted internally (264) and at least one accelerometer 286 may be deployed externally (266).

FIG. 3A is a diagram including a side view, schematically representing an example method 300 and/or example device including a sensor 304A. As shown in FIG. 3A, in some examples sensor 304A is chronically, subcutaneously implanted within a chest wall 302A of a patient’s body, and sensor 304A may comprise an acceleration sensor. During breathing, the chest wall 302A will exhibit rotational movement (B2) as at least some portions of the chest wall 302A move (e.g. rise and fall) during inspiration and expiration, wherein inspiration corresponds to expansion of the rib cage and expiration corresponds to contraction of the rib cage. During this expansion and contraction of the rib cage during breathing, at least some portions of the chest wall 302A exhibit rotational behavior, which may in turn may be sensed upon the sensor 304A experiencing such rotational movement (as represented via directional arrow B1), which in turn provides respiration information as further described below. It will be understood, as further described later, that the rotational movement of the sensed portion of the chest wall 302A is not necessarily or strictly limited to rotational movement in a single plane.

In some examples, sensor 304A may comprise a portion of a larger device, as the previously described implantable medical device 283 in association with at least FIG. 2B.

As further shown in FIG. 3A, the sensor 304A may sense the rotational movement of at least a portion of the chest wall 302A (as represented via directional arrow B2) relative to an earth gravitational field (arrow G), i.e. gravity vector. For illustrative simplicity to depict at least some examples, FIG. 3A depicts the chest wall 302A as if the patient’s body was in a generally horizontal sleep position. It will be understood that at least some example devices and/or example methods will be effective in detecting respiration information regardless of whether the generally horizontal sleep position is a supine position, a prone position, or a side-laying (i.e. lateral decubitus) position. Moreover, at least some example methods and/or example devices also will be effective in detecting respiration information if, and when, the patient is in positions other than a generally horizontal position, such as sitting in a chair in a vertically upright position, in a reclining position, etc.

Moreover, in some examples, determining respiration information via acceleration-based sensing of rotational movements (at a portion of a chest wall of the patient) does not include, or depend on, determining (e.g. via sensing) a body position of the patient. Accordingly, while such respiration information may be determined in any one of several different sleeping body positions, such determination may be performed without determining the particular sleeping body position at the time the sensing of the rotational movements is being performed.

However, in some such examples, the particular sleeping body position occurring at the time of the determining the respiration information (via acceleration-based sensing of rotational movements of a portion of a chest wall during breathing) may be determined and used as a parameter to augment the determined respiration information and/or other general patient physiologic information, in some instances.

In some examples, securing the implantable acceleration sensor(s) comprises mechanically coupling the sensor(s) relative to a respiratory body portion. In some examples, securing the implanted acceleration sensor(s) comprises securing the acceleration sensor relative to tissue on top of a muscle layer of the respiratory body portion, while in some examples the sensor may be secured directly to a muscle layer of the respiratory body portion. In some examples, the acceleration sensor may be secured subcutaneously within the respiratory body portion without securing the acceleration sensor on the muscle layers. In some examples, the respiratory body portion may comprise the chest. In some such examples, the respiratory body portion may comprise a portion of the chest, such as but not limited to a portion of a chest wall. In some instances, the portion of the chest wall may correspond to a portion of the rib cage. In some examples, such aspects of securing the sensor(s) relative to a muscle layer or subcutaneously are also applicable to securing the sensor at other respiratory body portions, such as an abdomen (e.g. abdominal wall) physically (e.g. mechanically) couple the sensor relative to the abdomen to sense rotational movement at the abdomen during breathing.

FIG. 3B is a diagram 320, including a side view, schematically representing an example method and/or example sensor 304A. As shown in FIG. 3B, in some examples the sensor 304A may comprise a sensing element 322A, which is arranged to measure an inclination angle (Ω) upon rotational movement of the sensing element 322A caused by breathing. As represented in FIG. 3B, upon rotational movement of at least a portion of the chest wall 302A during breathing, the sensing element 322A may rotationally move between a first angular orientation YR1 (shown in solid lines) and a second angular orientation YR2 (shown in dashed lines). In some such examples, the first angular orientation YR1 (shown in solid lines) of sensing element 322A may correspond to a peak expiration of a respiratory cycle (e.g. rib cage contracted) and the second angular orientation YR2 (shown in dashed lines) of sensing element 322A may correspond to a peak inspiration of the respiratory cycle (e.g. rib cage expanded).

With reference to at least FIG. 3B, it will understood that the sensing element 322A moves with at least a portion of the chest wall 302A as depicted in dashed lines. Accordingly, sensing element 322A does not move relative to the chest wall 302A, but rather the sensing element 322A rotationally moves along with (e.g. in synchrony with) the rotational movement of at least the portion of the chest wall 302A (in which the sensor 304A, including sensing element 322A), is implanted) during breathing.

In some examples, the sensing element 322A comprises an accelerometer, which may comprise a single axis accelerometer in some examples or which may comprise a multiple-axis accelerometer in some examples. Via the accelerometer, the sensing element 322A can determine absolute rotation of sensor 304A (and therefore rotation of the portion of the chest wall 302A) with respect to gravity (e.g. earth gravity vector G), rather than instantaneous changes in rotation.

In some examples, element 322A may comprise a single axis accelerometer to measure (at least) a value of, and changes in the value of, the above-noted inclination angle (Ω) associated with movement of at least a portion the chest wall 302A caused by breathing. In some examples, sensing element 322A may comprise an accelerometer and a gyroscope. In some examples, the sensing element 322A may comprise a multi-axis accelerometer.

FIG. 3C is a diagram including a graph 340 schematically representing a filtered acceleration signal 342 sensed via a sensor, such as sensing element 322A in FIG. 3B. As shown in FIG. 3C, signal 342 corresponds to a respiratory waveform exhibited through several respiratory cycles during breathing. Each respiratory cycle 343 comprises an inspiratory phase (Ti), an expiratory active phase (T_{EA}), and an expiratory pause phase (T_{EP}). It will be understood that the example respiratory waveform in FIG. 3C represents a typical respiratory waveform for at least some patients during normal breathing, but not necessarily for all patients at all times. In some examples, one full respiratory cycle comprises one full breath.

With FIG. 3C in mind, and with further reference to FIG. 3B, it will be noted that the first angular orientation YR1 of sensing element 322A (shown in solid lines) may correspond generally to a peak expiration 346 (e.g. end of the expiratory active phase (TEA)) while the second angular orientation YR2 of sensing element 322A (shown in dashed lines) may correspond to a peak 348 of inspiratory phase (TI), i.e. the end of inspiration just at or before the onset of expiration.

With further reference to FIG. 3B, upon rotation (B2) of at least a first portion of chest wall 302A, as represented by directional arrow B2 and the depiction of chest wall in dashed lines 302B, such as during inspiration, the sensing element 322A rotates by the inclination angle (Ω) with chest wall 302A to a position or orientation YR2 shown in dashed lines 322B. Upon the end of inspiration, and the ensuing expiration, the chest wall 302A will rotate back into the position shown in solid lines (e.g. end of expiration) such that the sensing element 322A will sense a change in inclination angle (Ω) from the position YR2 (shown in dashed lines) back to the position YR1 (shown in solid lines).

In sensing the inclination angle (Ω) through successive respiratory cycles, the sensing element 322A obtains an entire respiratory waveform, which may comprise information such as the duration, magnitude, etc. of an inspiratory phase (TI), expiratory active phase (TEA), and expiratory pause phase (TEP) of respiratory cycles of the patient, and/or other information (e.g. respiratory rate, etc.) as represented in FIG. 3C and/or as further described later. With this in mind, in some examples the obtained respiratory waveform (e.g. respiration morphology) also comprises a respiratory period, which includes the inspiratory phase, the expiratory active phase, and the expiratory pause phase. In one aspect, the respiratory period corresponds to a duration of a respiratory cycle, with this duration comprising a sum of a duration of the inspiratory phase, a duration of the expiratory active phase, and a duration of the expiratory pause phase.

In some examples, the identified respiration morphology comprises identifying (within the respiratory waveform morphology) a start of the inspiratory phase, i.e. an onset of inspiration. In some examples, this start of the inspiratory phase also may at least partially correspond to an expiration-to-inspiration transition. In some examples, a method of identifying the start of the inspiratory phase within the identified respiratory waveform morphology further comprises performing the identification (of the start of the inspiratory phase) without identifying an end (e.g. offset) of the inspiratory phase, thereby improving the accuracy of identification (of the start of the inspiratory phase) in the presence of noise, in contrast to identification of more than one phase transition (e.g. inspiratory-to-expiratory or expiratory-to-inspiratory) per respiratory cycle where each transition may be subject to mis-identification due to noise. In some such examples, the end (e.g. offset) of the inspiratory phase corresponds to a start (e.g. onset) of the expiratory active phase.

In some examples, identifying the respiratory waveform morphology may comprise identifying (within the respiratory waveform morphology) an respiratory peak pressure, which predictably occurs a short interval after the end of inspiration and which may be used in aspects of respiration detection and related parameters. In one aspect, this arrangement may enhance the accuracy of identification (of an inspiratory-to-expiratory transition, end of inspiration, etc.) in the presence of noise due to the ease of identification of the relatively high mathematical derivative of the pressure signal associated with the interval following the end of inspiration.

In some examples, the identification of respiratory waveform morphology may identify (within the respiratory waveform morphology) an end of expiration, which may be used in some aspects of respiration detection and related parameters.

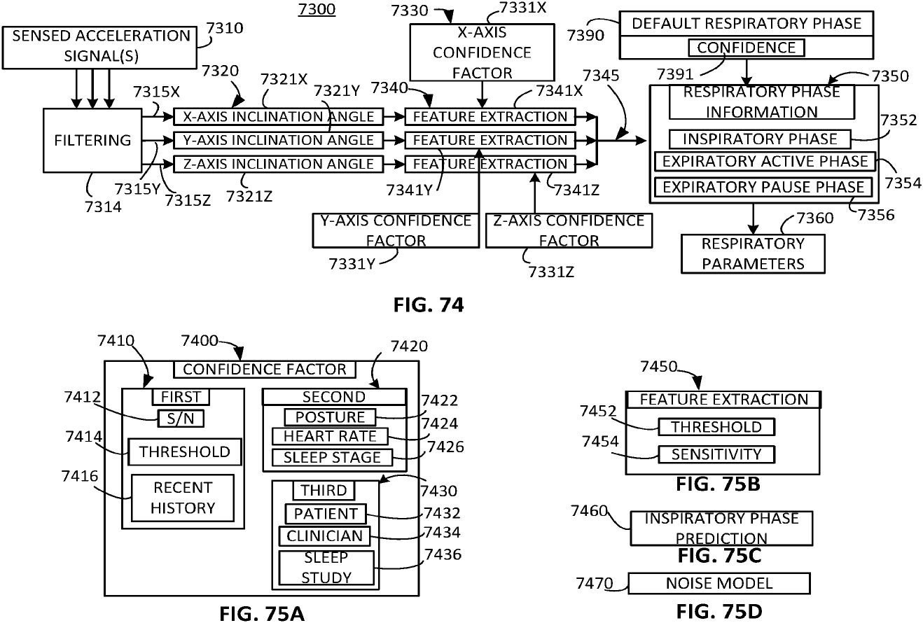

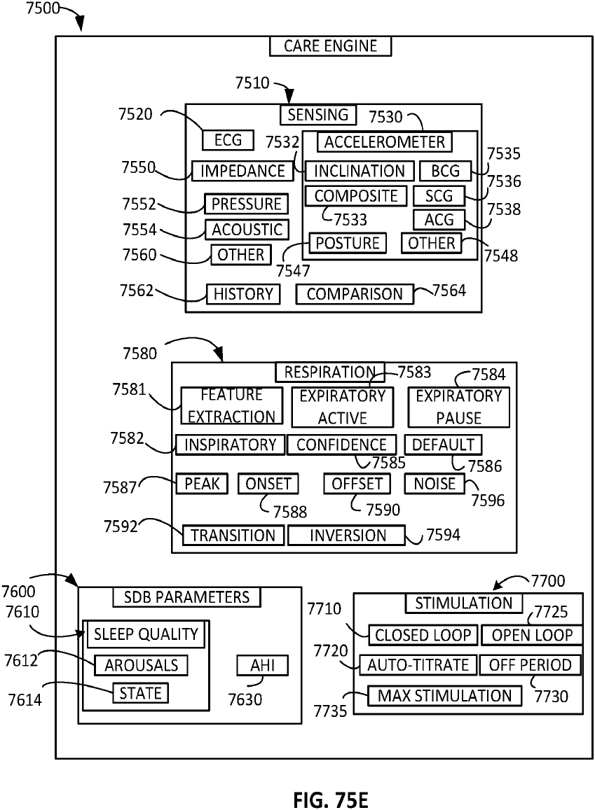

In some examples, at least some aspects of such identification, prediction, etc. of features (e.g. start of inspiratory phase, end of expiration, etc.) within a respiratory waveform may be implemented via at least some of substantially the same features and attributes as later described in association with at least FIGS. 74-75E and/or various examples throughout the present disclosure, such as but not limited to identifying inspiratory phase (e.g. 7352 in FIG. 74), inspiratory phase prediction (e.g. 7460 in FIG. 75C), etc.

With further reference to FIG. 3B, it will be understood that the second angular orientation YR2 of sensing element 322A is not a fixed position, but rather corresponds to a temporary position at one end (e.g. a second end) of a range of rotational movement of the portion of the chest wall 302A, such as peak inspiration 348 (FIG. 3C) during breathing. This second end of the range of rotational movement (and therefore the second angular orientation YR2) may vary depending upon whether the patient is exhibiting normal/relaxed breathing, forced breathing (such as more forceful inspiration), and/or disordered breathing. Moreover, this second end of the range of rotational movement may exhibit some variance from breath-to-breath even during relaxed breathing.

Similarly, the first angular orientation YR1 of sensing element 322A shown in FIG. 3B does not comprise a fixed position, but rather the first angular orientation YR1 corresponds to a temporary position at an opposite other end (e.g. a first end) of a range of rotational movement of the portion of the chest wall 302A, such as peak expiration 346 (FIG. 3C) during breathing. This first end of range of rotational movement (and therefore the first angular orientation YR1) may vary depending upon whether the patient is exhibiting normal/relaxed breathing, forced breathing (such as more forceful expiration), and/or disordered breathing. Moreover, this first end of the range of rotational movement may exhibit some variance from breath-to-breath even during normal/relaxed breathing.

While there may be some variances in the exact rotational positions of the respective second angular orientation YR2 and/or the first angular orientation YR1, it will be understood that there consistently will be a significant difference between the first angular orientation YR1 and the second angular orientation YR2, by which respiration morphology (e.g. shown in FIG. 3C) can be determined as the sensor moves through the full range of rotational movement between the first and second angular orientations, YR1 and YR2 during patient breathing. Moreover, the variances in the particular rotational position of the first angular orientation YR1, and of the second angular orientation YR2, at the ends of the range of rotational movement of the sensing element 122A may yield valuable information regarding variances in respiration, such as variances in amplitude of inspiration and/or expiration, variances in respiratory rate, etc. In some examples, the ends of the range of angular movement between the two orientations YR1, YR2 may correspond to the ends of a range of values of an AC signal component of the acceleration signal from the sensor.

In some examples, the first angular orientation YR1 may sometimes be referred to as a reference angular orientation, at least to the extent that the first angular orientation YR1 may correspond to an orientation which is the closest to being generally perpendicular to the gravity vector G for at least some sleeping body positions, such as but not limited to a generally horizontal sleep position.

With further reference to FIG. 3B, in this example implementation and in general terms, the sensor 304A may be implanted in a manner to cause the first angular orientation YR1 (i.e. base orientation) of the measurement axis of the sensing element 322A to be generally parallel to a superior-inferior (S---I) orientation of at least the chest region of the patient’s body, and generally perpendicular to an earth gravitational field G, such as when the patient is in a generally horizontal position. In this example implementation, the measurement axis of the sensing element 322A also may understood as having an orientation generally perpendicular an anterior-posterior (A --- P) orientation of at least a portion of the chest region of the patient’s body.

In the example of FIG. 3B, rotational movement of sensing element 322A, which has a Y-axis orientation, occurs roughly near or within a plane P1 defined by the anterior-posterior orientation (A—P) and by the superior-inferior orientation (S-I) of the patient’s body. This rotational movement is primarily indicative of rotational movement of the rib cage during breathing, such as during a treatment period in which a patient is sleeping. Additional examples later describe additional/other aspects in which rotational movements of the rib cage are further indicative of breathing, and therefore respiratory morphology.

It will be understood that, due to patient-to-patient variances in anatomy and/or due to the particular location where the sensor 304A is actually implanted along the chest wall 302A, the sensing element 322A may extend in an orientation which is not exactly parallel to a superior-inferior orientation the chest wall (or entire patient as a whole), and not exactly perpendicular to an anterior-posterior orientation of the patient’s body (and gravity vector G when laying in a generally horizontal position).

Nevertheless, in some example implementations, by arranging the measurement axis (Y) of the sensing element 322A to have an orientation as close as possible to being generally perpendicular to the gravity vector (G) for at least some patient body positions (e.g. generally horizontal sleep position), the sensitivity of the AC signal component of the acceleration sensing element 322A is maximized (and absolute value of the DC signal component of minimized), which in turn may increase the effectiveness of measuring changes in the inclination angle (Ω) of sensing element 322A caused by, and during, breathing by the patient. In one aspect, the AC signal component of the acceleration sensing element 322A may be understood as the time-varying portion of the output signal of the acceleration sensing element 322A.

In particular, by arranging the sensing element 322A within the chest wall 302A to be as close as reasonably practical to being generally perpendicular to the earth gravitational field G (at least when the patient is in a primary sleep position), the sensed inclination angle will correspond to a maximum value of a measured AC component of the acceleration signal and a minimum absolute value of measured DC component of the acceleration signal. Stated differently, when a measurement axis of the acceleration sensing element 322A is generally perpendicular (or as close as reasonably practical) to an orientation in which it would otherwise measure a maximum value (e.g. 1 g, such as when parallel to an earth gravity vector), the absolute value of the DC component will be negligible or minimal. In this situation, changes in value of the AC component of the acceleration signal become more prominent, being of a magnitude and/or reflecting significantly measurable changes as an orientation of the (measurement axis of the) acceleration sensing element changes (e.g. inclination angle) as the portion of the chest wall exhibits rotational movement caused by breathing.

It will be understood that based on the particular orientation at which the sensor 304A (e.g. sensing element 322A) is actually implanted, based on the varying position of the patient once the sensor 304A has been implanted, and/or based on other factors described further below, the measurement axis of the sensing element 322A at the chest wall 302A may not be perpendicular to the earth gravitational field G at the time of performing the sensing during breathing and hence the sensitivity of the AC component of the acceleration signal may not be at a maximum value. Nevertheless, at least some example methods (and/or devices) may perform the sensing (e.g. of the inclination angle of the sensing element 322A) to obtain the desired respiration information provided that the sensed signal provides a sufficiently high degree of sensitivity of a measured AC component of the acceleration signal. In some such examples, the methods and/or devices may employ magnitude criteria by which it may be determined if, and/or when, a sufficiently high degree of sensitivity of the measured AC signal is present. For instance, in some non-limiting examples, a sufficiently high degree of sensitivity corresponds to a measured AC signal having adequate signal to noise ratio in order to determine respiration.

In some such examples, an output acceleration signal of sensing element 322A corresponds to a sine of the angle between the accelerometer measurement axis (i.e. orientation of Y) and a generally horizontal orientation (which is generally perpendicular to gravity vector G).

Because of variances in patient-to-patient anatomy, in some example methods/devices, an absolute magnitude of the AC signal component is not used to determine respiration information. Rather, by using the difference in magnitude of the value of the AC signal component between the first angular orientation (YR1) and the second angular orientation (YR2), the example methods/devices can determine a respiratory waveform, morphology, etc.

In some examples, depending on the particular angle at which the device and sensor are implanted in a particular patient, and/or depending on the particular sleeping position in which the patient is arranged, the inspiration identified from the sensed respiratory waveform may have a positive slope or may have a negative slope. In some such examples in which the positive slope may be considered a default or primary mode, the negative slope may be considered to an inverted signal or exhibiting inversion of the respiratory waveform signal. Accordingly, in some examples, the example device/method may comprise a component such as slope inversion parameter 7594 in FIG. 75E for accounting the particular slope of the inspiratory phase of the respiratory waveform exhibited during sensing the signal, such as when a signal inversion may take place. In some such examples, the positive slope or the negative slope of the inspiratory phase may sometimes be referred to as a polarity of the slope of the inspiratory phase. It will be understood that in accounting for the particular slope of the inspiratory phase, the slope of the other phases of the respiratory cycle will be accounted for as well.

With these features in mind regarding the slope of the inspiratory phase of the respiratory waveform signal, at least some of the example methods and/or devices of the present disclosure may accurately capture and determine respiratory information regardless of how the patient may be moving in space, e.g. regardless of the direction of the sensor rotation in space or regardless of rotation of the patient (including the sensor) with respect to gravity. Accordingly, the example methods and/or devices may produce accurate, reliable determination of respiration information.

Accordingly, at least some example methods comprise implanting sensor 304A (including sensing element 322A) in a manner to maximize sensitivity of the AC component of the sensed acceleration signal by establishing an orientation (e.g. YR1) of sensing element 322A which is closest to being generally perpendicular to the gravity vector G, for at least some body positions such as a common sleep position (e.g. generally horizontal). In some situations, the sensor 304A (including sensing element 322A) may be implanted in a position in which the sensitivity of the AC component of the sensed acceleration signal is not maximized but which is sufficient to effectively and reliably determine respiration information based on sensed rotational movement at a first portion of a chest wall (or other physiologic location as described below). In some such examples, a sufficient sensitivity of the AC component of the sensed acceleration signal may comprise having an adequate signal-to-noise ratio.

With further regard to the observation that the device (including the acceleration sensor(s)) may be implanted at various particular orientations (e.g. angles) which are not parallel an ideal reference orientation (e.g. superior-inferior), it will be understand that in some examples, the example methods/devices determine the respiration information (e.g. using acceleration-based sensing of rotational movement of a portion of a chest wall, etc.) without calibrating the measured inclination angle signal (of the acceleration sensor) relative to any difference between the ideal reference orientation (e.g. superior-inferior) and the actual implant orientation (as shown later at 7870 in FIG. 82). However, in some examples, such calibration may be performed and/or such differences may be considered in using the sensed information.

As noted elsewhere, in at least some examples, determining respiration information based on acceleration sensing of rotational movement (of a portion of the chest wall) does not depend on the sensor having an ideal implant orientation, does not depend on knowing the actual implant orientation, and/or does not depend on accounting for differences between the ideal implant orientation and the actual implant orientation.

In some later example implementations, a sensor comprises multiple sensing elements such that the example methods may comprise determining which of the multiple sensing elements has an orientation which is closest to being generally perpendicular to gravity vector, and therefore which may provide the most sensitivity and effectiveness in sensing respiratory information. In some such examples, the multiple sensing elements may be oriented orthogonally relative to each other or may be oriented at other angles (e.g. 45 degrees) relative to each other.

With further reference to at least FIG. 3C, in some examples, the term “generally perpendicular” may comprise the first angular orientation YR1 being at some angle relative to the gravity vector G (e.g. 85, 86, 87, 88, 89, 91, 92, 93, 94, 95 degrees) which varies slightly from an exactly perpendicular angle (e.g. 90 degrees) relative to the gravity vector G. Moreover, as noted above and/or further described below, the effectiveness of measuring respiration by changes in the inclination angle (Ω) between the first and second orientations (YR1, YR2) does not strictly depend on the first angular orientation YR1 being exactly perpendicular to the gravity vector G.

However, as further described later in association with at least FIGS. 66A-68, the first angular orientation YR1 may be at angles other than generally perpendicular relative to the gravity vector (G), such as in example implementations in which the first angular orientation YR1 of a sensing element (e.g. 322A) is positioned to be about 135 degrees relative to gravity vector G (i.e. 135 degrees to an anterior-posterior (A - P) orientation of patient’s body. In some such examples, the second angular orientation YR2 of sensing element 322A would still extend at an angle (Ω) relative to the first angular orientation YR1, with it being understood that angle (Ω) varies according to the variances in respiration of the patient which occur in normal breathing, forced breathing, and/or disordered breathing, as previously described. As further described later, establishing the first orientation TR1 at angles other than 135 degrees are contemplated as well.

As further described later in association with at least FIGS. 85-86, 7470 noise model in FIG. 75D, and/or noise parameter 7596 in FIG. 75E, the example device(s) and/or example method(s) may perform such measurements in a manner to exclude (e.g. filter) measurements of gross body motion, measurement noise, muscle noise, cardiac noise, other noise, etc. such that the remaining sensed or measured acceleration signal is primarily representative of movement of at least a portion of the chest wall 302A. In some such examples, the measured acceleration signal is representative solely of movement of the chest wall 302A. In particular, in some such examples, the measured acceleration signal corresponds to rotational movements of at least a portion of the chest wall 302A as sensed by sensor 304A (B1 in FIG. 3A) caused by and/or occurring during breathing.

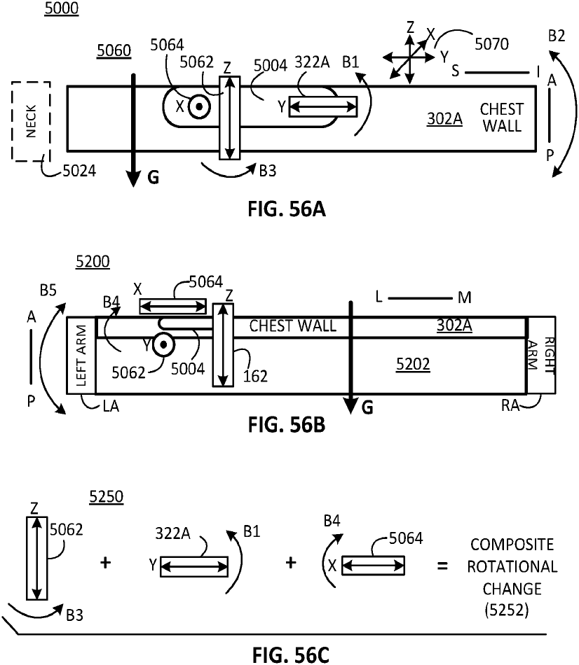

At least some further example implementations regarding sensing respiratory information and/or other physiologic information in relation to at least an acceleration sensor are described later in association with at least FIGS. 56A-102.

FIG. 4 is a block diagram, which may comprise part of a flow diagram in an example method (e.g. 100 in FIG. 1A, 240 in FIG. 2A). As shown at 450 in FIG. 4, the example method may comprise sensing the physiologic information and identifying the disease burden indicator upon a criteria being met by a quantity of disease burden indicator events and/or a rate of disease burden indicator events.

In some examples, the disease burden indicator in association with FIG. 4 may comprise sleep disordered breathing (SDB). In some such examples, the respiration information may be determined according to at least the example described in association at least FIGS. 3A-3C. In some examples, the rate of apnea/hypopnea events may be expressed via an apnea-hypopnea index (AHI).

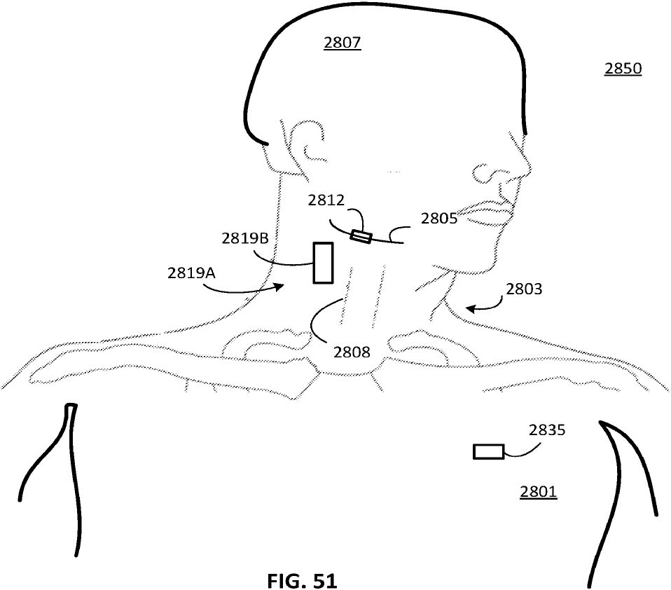

As further shown at 452 in FIG. 5, upon determining an indication of disease burden), a method (e.g. 100, 240) may comprise applying, via at least the implantable medical device (IMD) 283, therapy to treat a condition associated with the disease burden indicator. In some such examples, applying a therapy may comprising stimulating an respiration-related nerve (e.g. hypoglossal nerve, ansa cervicalis, phrenic, other) to treat the sleep disordered breathing (SDB). At least some examples of such stimulation are further described later in association with at least FIGS. 50-51.

In some example methods and as shown at 454 in FIG. 6, identification of the disease burden indicator may be implemented via a first control portion of the implantable medical device (IMD) 283. Various example implementations are further described later throughout the examples of the present disclosure.

In some example methods, and as shown at 460 in FIG. 7, a second control portion may be arranged external to the patient and in communication with the first control portion to at least partially implement disease burden identification in association with the implantable medical device.

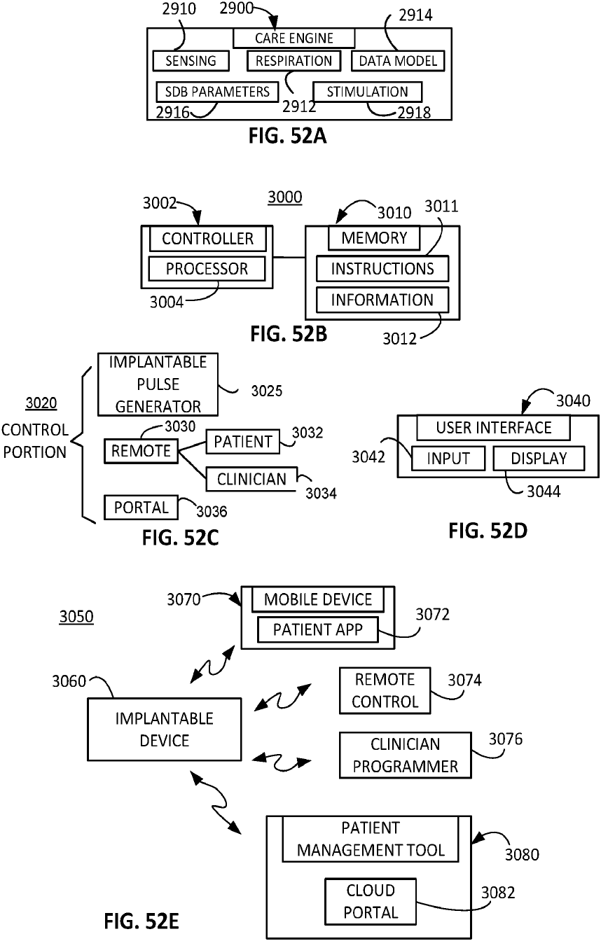

In some examples, the first control portion and/or second control portion may comprise at least some of substantially the same features as control portion 3000 described in association with at least FIGS. 52B-52E.

At least some example methods and/or devices may involve programming an IMD (e.g. 283 in FIG. 2B) to identify disease burden indicator(s) via an implantable sensor, such as an implantable acceleration sensor (e.g. 285, 304A, 322A), which may form part of or be associated with the IMD. In some examples, such programming may comprise determining which internally sensed physiologic information is correlated with, and/or acts as a surrogate for, externally sensed physiologic information typically used to identify the disease burden indicators (e.g. sleep disordered breathing (SDB), other). In some such examples, the programming may involve a control portion, such as the first and/or second control portion of the IMD.

In some examples, the first control portion and/or the second control portion may comprise a data model. However, as previously mentioned, in some examples, the first control portion may implement the identification of disease burden indicator(s) without use of a data model as part of the first control portion and/or second control portion.

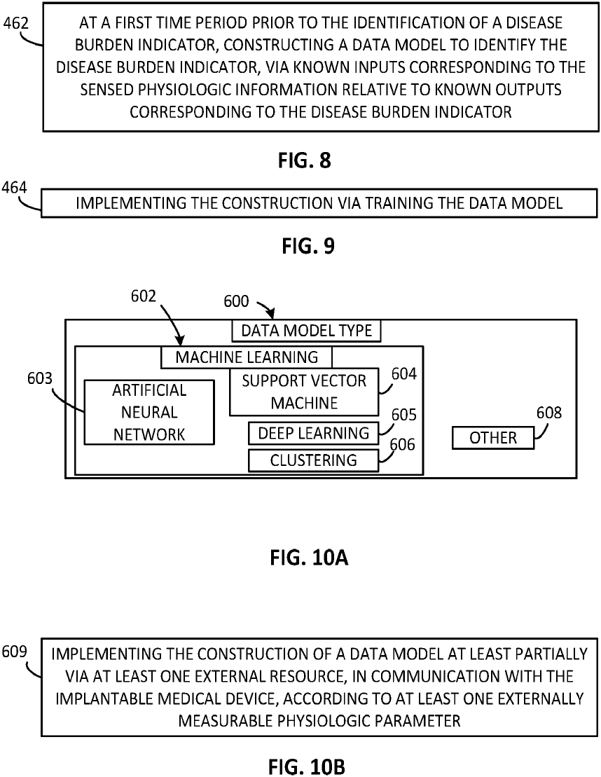

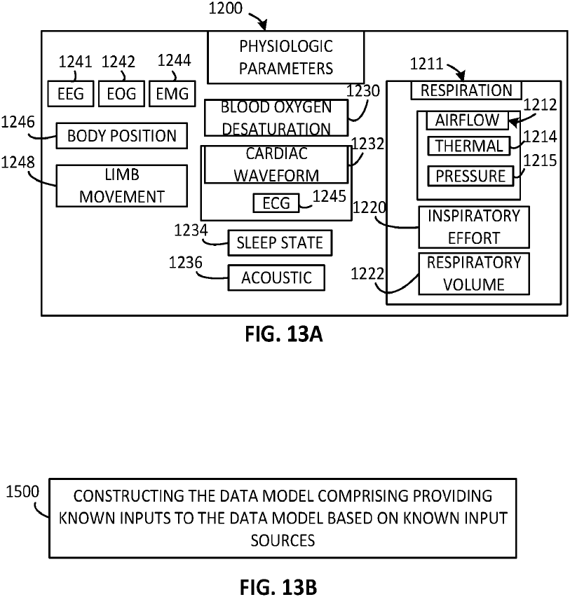

With this in mind, the following example implementations in FIG. 8-52E provide a framework of parameters, inputs, input sources, outputs, signals, devices, methods, etc., as part of providing an IMD to identify disease burden indication via internally sensed physiologic information, with at least some of the examples in FIGS. 13A-52A being particularly applicable to a sleep disordered breathing (SDB) indicator as an example disease burden indicator. Some of the example implementations comprise a data model or parameters, inputs, etc. associated with use of a data model, while some example implementations omit use of a data model. Regardless of whether a particular example includes a data model or not, it will be understood that the various parameters, inputs, input sources, signals, devices, methods may be combined in various permutations to achieve a desired array of inputs, outputs, etc. by which the IMD may be programmed or otherwise constructed to identify sleep disordered breathing (SDB) via internally sensed physiologic information.

While not necessarily indicating a preference for a data model implementation over non-data model implementations, this present disclosure will first address at least some aspects of the use of data models to program and/or construct an IMD to identify disease burden indication via internally sensed physiologic information.

Accordingly, in some example methods, and as shown at 462 in FIG. 8, at a first time period prior to the identification of a disease burden indicator (DBI), a data model may be constructed to identify the disease burden indicator (DBI) via known inputs corresponding to the sensed physiologic information relative to known outputs corresponding to the disease burden indicator (DBI). In some such examples, the data model may be constructed via training the data model, as shown at 464 in FIG. 9. In some such examples, the disease burden indicator (DBI) may comprise a sleep disordered breathing (SDB) indicator.

In some examples, the data model may comprise at least one of the data model types 600 shown in FIG. 10A. Accordingly, as shown in FIG. 10A, in some examples the data model types 600 may comprise a machine learning model 602, which may comprise an artificial neural network 603, support vector machine (SVM) 604, deep learning 605, clustering 606, or other model 608.

In some examples, the artificial neural network 603 may estimate a function(s) that depend on inputs. In some such examples, one or more layers of artificial neurons may receive input data and generate output data. The inputs and outputs can comprise physiological data and/or functions related to such physiologic data or other functions. Neural networks can comprise networks such as, but not limited to, learning networks (e.g. deep, deep structured, hierarchical, and the like), convolutional, auto-type networks (e.g. auto-encoder, auto-associator), Diablo networks, and neural network models (e.g. feedforward, recurrent).

In some examples, the support vector machine (SVM) 604 may utilize a linear classification. This classification can act to separate physiological data points into classes based on distance of the data points from a hyperplane. In some examples, the hyperplane is arranged to maximize the distances from the hyperplane to the nearest data points on either side of the hyperplane. This arrangement may group points located on opposite sides of the hyperplane into different classes. However, in some examples, the SVM may comprise a nonlinear classification that separates the data points with a hyperplane in a transformed feature space. The transformed feature space can be determined by one or more kernel functions, including nonlinear kernel functions. In some examples, the SVM is a multiclass SVM that separates data points into more than two classes, which may reduce a multiclass problem into multiple binary classification problems.

In some examples, the deep learning model 605 may comprise models such as, but not limited to, convolutional networks (e.g. deep belief, neural), belief networks, Boltzmann machines, deep coding networks, stacked auto-encoders, stacking networks (e.g. deep or tensor deep), hierarchical-deep models, deep kernel machines, and the like. It will be understood that such examples may comprise variants and/or combinations of the above-noted example networks.

In some examples, per type 606, the data model may comprise a clustering method(s), which may comprise hierarchical clustering, k-means clustering, density-based clustering, and the like. In some examples, the hierarchical clustering can be used to construct a hierarchy of clusters of physiological data. In some such examples, the hierarchical clustering utilizes a “bottom up” approach (e.g. agglomerative) wherein each data point starts in its own cluster, and pairs of clusters are merged at progressively higher levels of the hierarchy. However, in some examples, the hierarchical clustering utilizes a top-down approach in which all data points start in one cluster, and then clusters are split at progressively lower levels of the hierarchy.

In some examples, the k-means clustering implementation may comprise placing the sensed physiological data into k clusters, where k is an integer equal or greater than two. Via such clustering, each data point belongs to a cluster having a mean that is closer to the data point than any means of the other clusters. However, in some examples, a machine learning model (MLM) may comprise density-based clustering, which may be used to group together physiological data points that are close to one another, while identifying as outliers any data points that are far away from other data points.

In some examples, as represented per “other” type 608 in FIG. 10A, a machine learning model (MLM) may comprise a mean-shift analysis that can be used to determine the maxima of a density function based on discrete physiological data sampled from that function.

In some examples, as represented per “other” type 608 in FIG. 10A, a machine learning model (MLM) may comprise structured prediction techniques and/or structured learning techniques. Such techniques may be used to predict structured objects and/or structured data, such as structured physiological data. In some such examples, such structured prediction and/or structured learning techniques can comprise graphical models, probabilistic graphical models, sequence labeling, conditional random fields, parsing, collective classification, bipartite matching, Bayesian networks or models, and the like. It will be understood that such examples comprise variants and/or combinations of the above-noted example techniques.

In some examples, a machine learning model (MLM) may comprise anomaly detection and/or outlier detection that can be used to identify physiological data that do not conform to an expected pattern or are otherwise distinct from other physiological data in a dataset.

In some examples, machine learning model may comprise learning methods that incorporate a plurality of the machine learning methods.

It will be understood that at least some example methods (and/or devices) of the present disclosure may sense respiration and/or other physiologic information, and determine sleep disordered breathing (SDB), blood oxygen desaturation, etc. without use of a constructed data model and/or trained data model, such as but not limited to, a machine learning model.

As shown at 609 in FIG. 10B, in some examples a method may comprise implementing construction of a data model at least partially via at least one external resource, in communication with an implantable medical device (IMD) 283, according to at least some measurable physiologic parameters. In some such examples, the physiologic parameters are externally measurable. In some examples, the external resource comprises at least one sensor and/or a device, portal, etc. which receives information from a sensor regarding sensed measurable physiologic parameter.

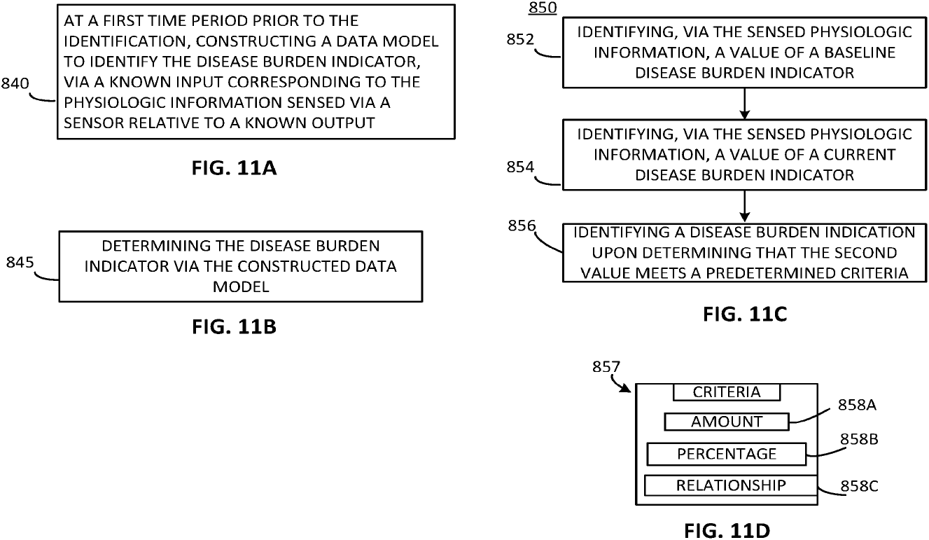

FIG. 11A is a diagram 840 schematically representing an example method 840 and/or device which may be employed to implement example methods (e.g. 100 in FIG. 1A, 240, in FIG. 2A, etc.) of identifying disease burden indicators. As shown at 840 in FIG. 11A, one example method comprises at a first time prior to identifying a disease burden indicator, constructing a data model adapted to identify a disease burden indicator, via known inputs corresponding to physiologic information sensed via a sensor relative to known output(s). The sensor may comprise an implantable sensor, while in some examples the sensor may comprise an implantable sensor and/or external sensor. In some examples, the known output(s) may comprise a disease burden indicator and/or a measurable physiologic parameter, which may be associated with a disease burden indicator. In some examples, the measurable physiologic parameter may be measurable via external sensors, elements, while in some examples, the measurable physiologic parameter may be measurable via external sensors, implantable sensors, and/or removably insertable internal sensors.

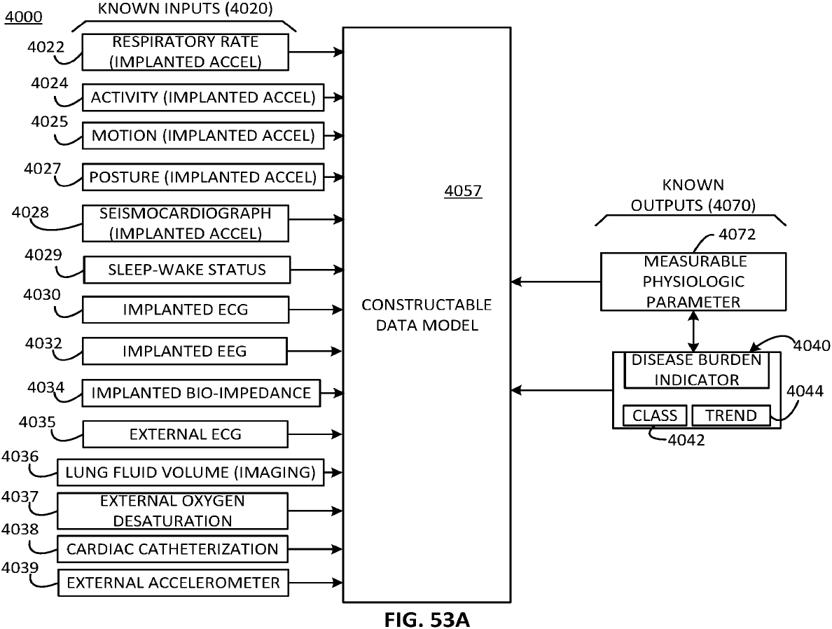

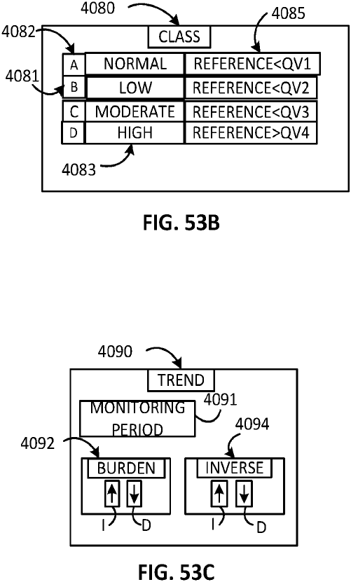

Upon construction of the data model per method 840 in FIG. 11A, method 845 in FIG. 11B comprises determining the disease burden indicator via the constructed data model. In some examples, the disease burden indicator may comprise a quantitative value, which may be compared to a reference. In some examples, the determined disease burden indicator also may be compared to a plurality of classes of the disease burden indicator and/or may be evaluated regarding trend information, as further described later in association with at least FIGS. 53A-55C.

FIG. 11C is a flow diagram schematically representing an example method 850, which may comprise one example implementation of the example method 845 (FIG. 11B) or which may comprise an example method implementable without use of a data model. As shown at 852 in FIG. 11C, method 850 may comprise identifying, via the sensed physiologic information, a value of a baseline disease burden indication. As shown at 854 in FIG. 11C method 850 further comprises identifying, via the sensed physiologic information, a value of a current disease burden indicator and at 856, the method comprises identifying a disease burden indication upon the second value meeting a predetermined criteria. In some examples, as shown in FIG. 11D, the predetermined criteria 857 may comprise an amount 858A, a percentage difference 858B, and/or a relationship (e.g. ratio) 858C between the first and second values.

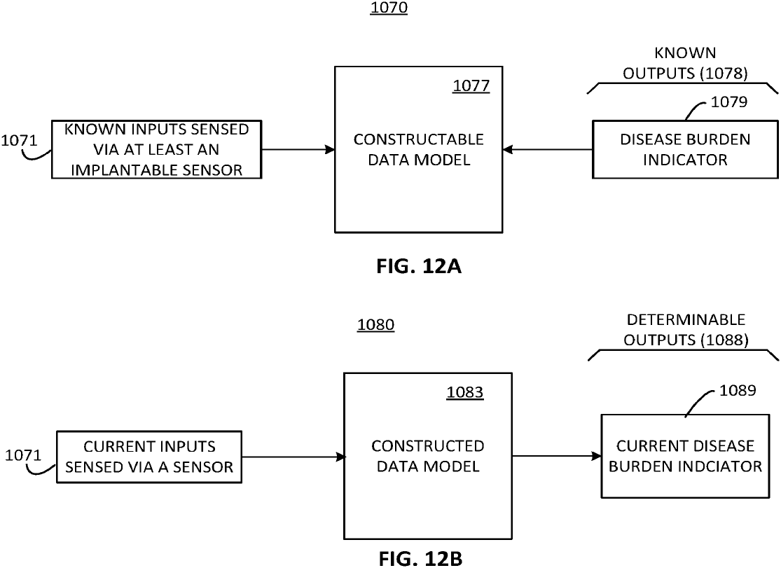

FIG. 12A is diagram schematically representing an example method 1070 (and/or device) to construct a data model 1077. In some examples, method 1070 may comprise at least some of substantially the same features and attributes of, and/or an example implementation of, the examples previously described in association with at least FIGS. 1A-11D. As shown in FIG. 12A, method 1070 comprises constructing data model 1077 using known inputs 1071 and known output(s) 1078. The known inputs 1071 may be obtained via an implantable sensor while in some examples, the known inputs 1071 may be obtained via an implantable sensor and/or a sensor located external to the patient’s body. In some examples, the known outputs 1078, 1079 may be obtained via an external sensor while in some examples, the known outputs 1078, 1079 may be obtained via an external sensor and/or a sensor insertable within the patient’s body.

Once constructed as shown in FIG. 12A, a data model 1083 may be used in an example method 1080, as shown in FIG. 12B, in which currently sensed inputs 1081 are fed into the constructed data model 1083, which produces an output 1088 as a current disease burden indicator 1089. In some examples, the current inputs 1081 comprise information sensed solely via an implantable sensor while in some examples, the current inputs 1081 comprise information sensed via an implantable sensor and/or an external sensor.

The examples of FIGS. 1A-12B are applicable to identifying disease burden for a wide variety of diseases, just one of which may comprise sleep disordered breathing. Accordingly, while the following examples in FIGS. 13A-52A may primarily involve sleep disordered breathing, it will be understood that at least some features and attributes of these examples may be applicable to other diseases. Moreover, the examples described later in association with at least FIGS. 53A-55C provide at least some specific examples relating to diseases other than sleep disordered breathing. In addition, while the examples described later in association with at least FIGS. 56A-102 may relate primarily to detecting respiration in the context of detecting and/or treating sleep disordered breathing, it will be understood that at least some features and attributes of those examples may be applicable to identification of disease burden in relation to FIGS. 53A-55C, as well as in relation to FIGS. 1A-52D.

FIG. 13A is a block diagram schematically representing at least some example externally measurable physiologic parameters 1200. In at least some examples, these physiologic parameters 1200 may be used to construct a data model, identify a disease burden indicator, etc., which may relate to sleep disordered breathing and/or other diseases. In some examples, these parameters 1200 may comprise respiration parameters 1211. In some such examples, the respiration parameters 1211 may comprise a respiratory airflow parameter 1212, which may comprise a thermal parameter 1214, and/or a respiratory pressure parameter 1215. In some examples, the respiration parameter 1211 may comprise an inspiratory effort parameter 1220 and/or a respiratory volume parameter 1222. In some examples, the respiration parameter 1211 may involve more general measures of respiratory effort, which may include inspiratory effort.

In some examples, the physiologic parameters 1200 may comprise a blood oxygen desaturation parameter 1230, a cardiac waveform parameter 1232, a sleep state parameter 1234, and/or an acoustic parameter 1236. The cardiac waveform parameter 1232 may comprise an electrocardiography (ECG) parameter 1245, in some examples. In some examples, the sleep state parameter 1234 may determine and/or track a patient sleep-wake status, and if the patient is sleeping, may determine and/or track sleep stages (e.g. N1, N2, N3, REM). In some examples, the blood oxygen desaturation information (1230) may be obtained via pulse oximetry. In some examples, the acoustic parameter 1236 may sense snoring and/or other patient sounds.

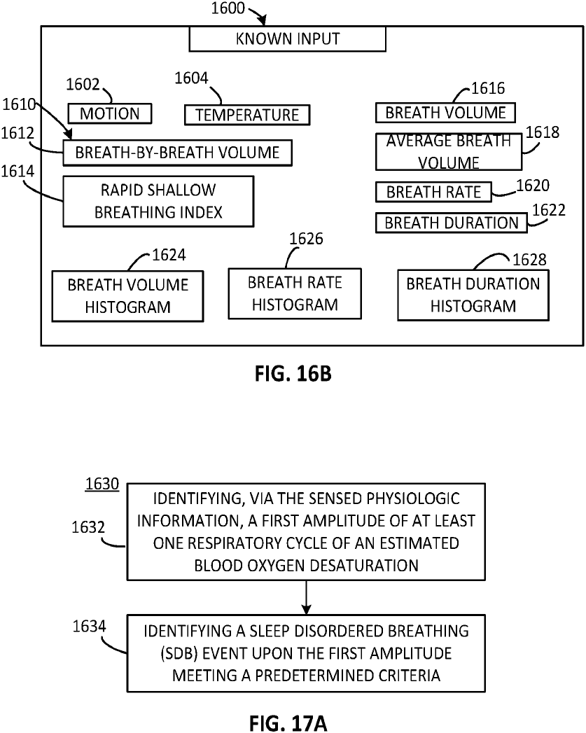

In some examples, the physiologic parameters 1200 may comprise an electroencephalography (EEG) parameter 1241, an electroocoulogram (EOG) parameter 1242, and/or an electromyography (EMG) parameter 1244. In some examples, the physiologic parameters 1200 may comprise a body position parameter 1246 and/or a limb movement parameter 1248. In some examples, the body position parameter 1246 (e.g. a body position signal) may be obtained via an implanted accelerometer (e.g. 285, 304A, 322A in FIGS. 2B-3C). The limb movement signal 1248 may be obtained via EMG measurements and/or computer vision. In some examples, the EMG signal 1244 may comprise EMG information obtained at or via a chin of the patient. It will be understood that the representation of physiologic parameters 1200 does not exclude other externally measurable physiologic parameters. In some examples, at least some of the parameters 1241, 1242, 1244, 1246 and/or 1248 may utilized to identify an arousal as further described in association with at least FIGS. 16A, 16B, and 32A-34.

As shown at 1500 in FIG. 13B, in some example methods a data model may be constructed via providing known inputs to the data model based on known input sources. In some examples, the input sources may comprise and/or support at least one of the physiologic parameters 1200 (FIG. 13A).

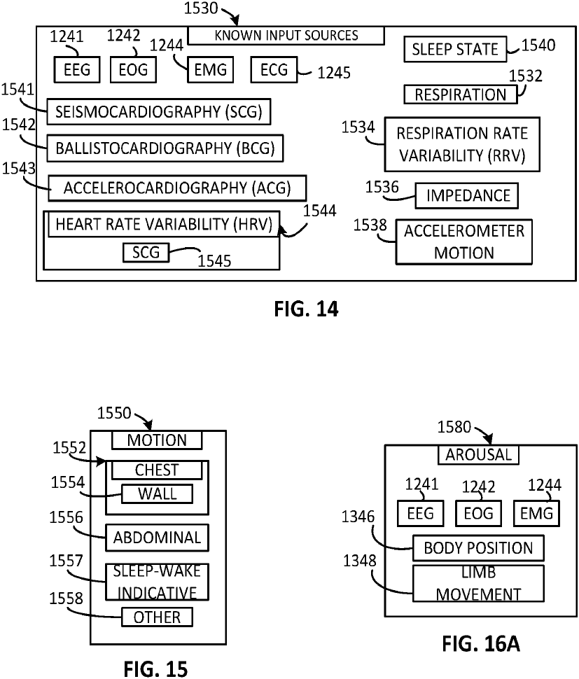

Moreover, as shown in FIG. 14, in some examples, the known input sources 1530 may comprise a respiration signal 1532, a respiration rate variability signal 1534, an impedance signal 1536 (e.g. lead impedance), and/or an accelerometer motion signal 1538. The accelerometer motion signal 1538 may be based on sensing via accelerometer 285, via sensing elements 304A, 322A (FIGS. 2B-3B). In some such examples, the impedance signal 1536 may comprise various bioimpedance vectors, measurement waveforms, etc. In some examples, the bioimpedance may comprise a trans-thoracic bioimpedance. In some examples, the bioimpedance may be obtained via separate impedance sensors spaced apart on the patient’s body. In some such examples, the separate impedance sensors may comprise a portion of a lead body, a sensing element, and/or a stimulation element, etc.

As further shown in FIG. 14, in some examples known input sources 1530 may comprise an EEG parameter 1241, EOG parameter 1242, an EMG parameter 1244, and/or an ECG parameter 1245, such as in FIG. 13A.

As further shown in FIG. 14, in some examples the known input sources 1530 may comprise seismocardiography sensing 1541 (SCG), ballistocardiography sensing (BCG) 1542, and/or accelerocardiograph sensing (ACG) 1543. In some examples, the SCG, BCG, ACG sensing may be provided via an implanted accelerometer (e.g. 285, 304A, 322A) or via other types of implantable sensing elements. As further shown in FIG. 14, in some examples, the known input sources 1530 may comprise a heart rate variability (HRV) signal 1544, which in some examples may be obtained from SCG sensing 1545.

As previously noted, these known inputs in FIG. 14 may be used to detect respiration and parameters relating to respiration, sleep disordered breathing, and/or disease burden indicators for other diseases.

FIG. 15 is a block diagram schematically representing example accelerometer motion 1550, which may comprise example implementations of the accelerometer motion 1538 in FIG. 14 in some examples. As shown in FIG. 15, in some examples the accelerometer motion 1550 may comprise a chest motion 1552. In some such examples, the chest motion 1552 comprises a chest wall motion 1554. In some examples, the chest wall motion 1554 comprises a rotational movement of the chest wall as described in association with at least FIGS. 3A-3C and/or at least FIGS. 56A-95.

In some examples, the accelerometer motion 1550 may comprise an abdominal motion 1556, which comprise a rotational movement of an abdominal wall or portion of the abdomen indicative to respiratory information. In some examples, the rotational movement of the abdomen (or abdominal wall) may comprise at least some of substantially the same features and attributes as the abdominal motion and detection described in association with at least FIGS. 3A-3C and/or FIGS. 56A-102.

As further shown in FIG. 15, in some examples the accelerometer motion 1550 may comprise a sleep-wake indicative parameter 1557 by which a sleep-wake status of the patient may be determined.

In some examples, the accelerometer motion 1550 may comprise other parameters 1558 obtained, derived, etc. from the sensed motion via the accelerometer.

In some examples, some sensed physiologic information may be used in addition to, or instead, of the accelerometer motion (FIG. 15) as part of constructing a data model, identifying a disease burden indicator, determining sleep quality, and/or confirming sleep disordered breathing, etc. With this in mind, FIG. 16A is a block diagram schematically representing an example arousal input source 1580, which in general terms, provides various input sources by which an arousal may be detected and/or determined. In some examples, these input sources 1580 may be used to construct a data model. In some examples, the arousal input source 1580 may comprise an EEG signal 1241, EOG signal 1242, EMG signal 1244, a body position signal 1346, and/or a limb movement signal 1348, each of which may comprise at least some of substantially the same features and attributes as previously described in association with at least FIG. 13A.

In some examples, constructing a data model comprises the use of additional known inputs and/or other known inputs. Accordingly, FIG. 16B is block diagram schematically representing at least some example known inputs 1600 for use in constructing (e.g. training) a data model and/or otherwise programming or calibrating a control portion (e.g. 3000 in FIG. 52B) to identify a disease burden indicator, such as but not limited to, sleep disordered breathing (SDB) based on internally sensed (e.g. accelerometer) physiologic information.

As shown in FIG. 16B, in some examples the known inputs 1600 comprise a motion input 1602, such as but not limited to, the accelerometer motion 1550 (FIG. 15), accelerometer motion 1538 (FIG. 14), respiration 1532, etc. in FIG. 14.

In some examples, the known inputs 1600 in FIG. 16B comprise temperature 1604, which may be sensed via an implanted accelerometer (e.g. 285, 304A, 322A) and/or a non-acceleration based temperature sensor. In some such examples, the known inputs 1600 comprise a combination of the above-described accelerometer motion 1602 and temperature 1604.

In some examples, the known inputs 1600 comprise an array of breath-related inputs 1610, such as but not limited to: a breath-by-breath volume 1612; a rapid shallow breathing index 1614; a breath volume 1616; an average breath volume 1618; a breath rate 1620; a breath duration 1622; a breath volume histogram 1624; a breath rate histogram 1626; and a breath duration histogram 1628.

In view of the foregoing example implementations providing a general framework of various parameters, inputs, input sources, data models, etc. which can be used to program or construct an IMD (to identify disease burden indicator (e.g. sleep disordered breathing) via internally sensed physiologic information), at least the following examples provide some more specific example implementations.

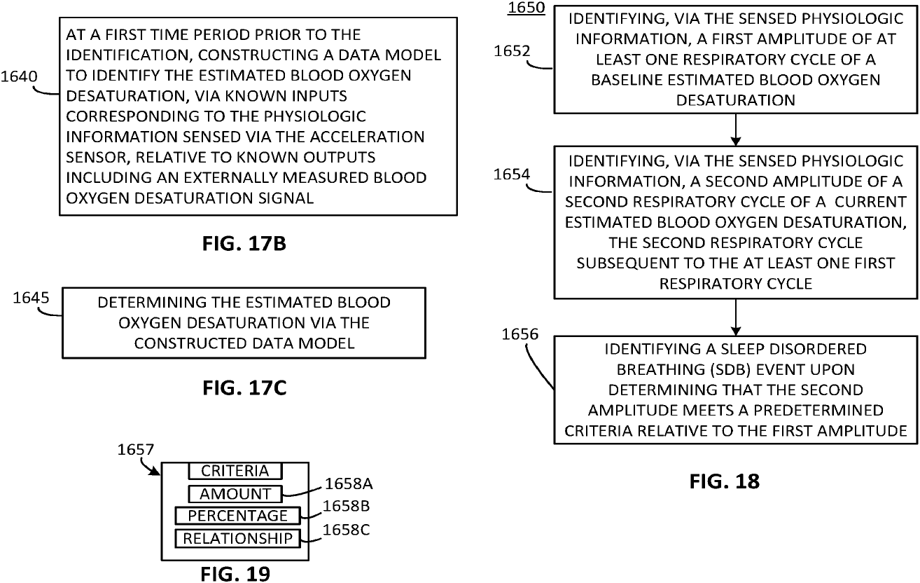

With this in mind, FIG. 17A is a flow diagram schematically representing an example method 1630 of identifying a sleep disordered breathing based on blood oxygen desaturation. As shown at 1632 in FIG. 17A, the method 1630 may comprise identifying, via the sensed physiologic information, a first amplitude of at least one respiratory cycle (e.g. at least one breath) of an estimated blood oxygen desaturation. At 1634, method 1630 comprises identifying sleep disordered breathing upon determining that the first amplitude meets a predetermined criteria. It will be understood that in some examples, the blood oxygen desaturation information may be used to determine a disease burden indicator for diseases other than sleep disordered breathing.

In some examples, the predetermined criteria may comprise a selectable amplitude criteria, such as a threshold amplitude (e.g. percentage) of blood oxygen desaturation, such as 94%, 93%, 92%, 91%, 90%, and the like. In some examples, the predetermined criteria may comprise a selectable duration (e.g. 10 seconds or other time period) or frequency that the first amplitude meets the amplitude criteria.

In some examples, the predetermined criteria comprises at least a 3 percent change in amplitude of a current estimated blood oxygen desaturation signal relative to a baseline estimated blood oxygen desaturation signal, where the term “baseline” refers to generally normal breathing. In some examples, in referring to generally normally breathing, the baseline signal corresponds to stable breathing which is generally free of sleep disordered breathing events, and as such, exhibits stable blood oxygen desaturation, stable respiratory airflow, and/or generally stable inspiratory effort.

In some examples, the predetermined criteria comprises at least a 4 percent change in amplitude of an estimated blood oxygen desaturation (e.g. current compared to baseline). In some examples, the predetermined criteria is selectable and implemented via a control portion (e.g. 3000 in FIG. 52B) and/or care engine 2900 (FIG. 52A). At least some details regarding the predetermined criteria regarding blood oxygen desaturation are further described below.

In some examples, the method 1630 in FIG. 17A may be implemented based on a data model. Accordingly, as shown at 1640 in FIG. 17B, method 1630 may further comprise, at a time period prior to the identification the first amplitude of an estimated blood oxygen desaturation, constructing a data model to identify the estimated blood oxygen desaturation. In some such examples, the construction may be implemented via known inputs corresponding to the physiologic information sensed via the acceleration sensor, relative to known outputs, such as but not limited to, an externally measured blood oxygen desaturation signal. In some such examples, the method 1640 may comprise using pulse oximetry to perform externally measuring blood oxygen desaturation.

In some examples, the method at 1640 in FIG. 17B may comprise part of method 1630 (FIG. 17A) or be a standalone method.

Upon constructing the data model at 1640 in FIG. 17B, method 1630 may further comprise determining the estimated blood oxygen desaturation (e.g. at 1632 in FIG. 17A) via the constructed data model, as shown at 1645 in FIG. 17C.

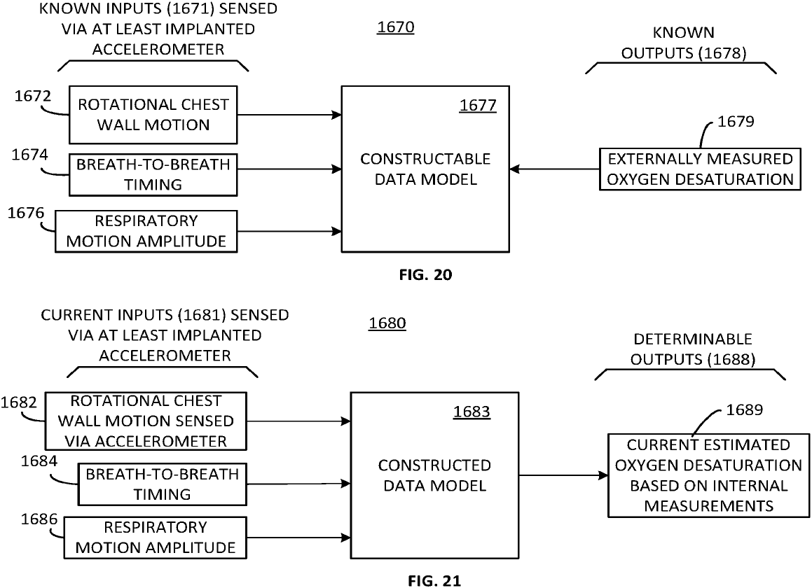

At least some details regarding constructing the data model are further described below in association with at least FIGS. 20-23.

FIG. 18 is a flow diagram schematically representing an example method 1650 of identifying a sleep disordered breathing based on blood oxygen desaturation. In some examples, method 1650 comprises a more detailed implementation of method 1630 in FIG. 17A. As shown at 1652 in FIG. 18, the method 8650 may comprise identifying, via the sensed physiologic information, a first amplitude of at least one respiratory cycle of a baseline estimated blood oxygen desaturation signal. At 1654, the method 1650 comprises identifying, via the sensed physiologic information, a second amplitude of a second respiratory cycle of a current estimated blood oxygen desaturation, with the second respiratory cycle being subsequent to the at least one first respiratory cycle. At 1656, the method 1650 comprises identifying sleep disordered breathing upon determining that the second amplitude differs from the first amplitude by a criteria. In some examples, as shown in FIG. 19, a criteria 1657 may comprise an amount 1658A, a percentage difference 1658B, and/or a relationship (e.g. ratio) 1658C between the first and second amplitudes.

FIG. 20 is a diagram schematically representing an example method 1670 of constructing a data model for use in later determining estimated blood oxygen desaturation. As shown in FIG. 20, known inputs 1671 sensed via at least an implanted accelerometer are provided to a constructable data model 1677 and a known output 1678 is provided to the constructable data model 1677. The known output 1678 may comprise an externally measured blood oxygen desaturation 1679, such as via pulse oximetry. As previously described in association with at least FIGS. 8-12B, constructing the data model may comprise training a data model, such as one of the data models in data model types 600 in FIG. 10A with one of the example data model types comprising a machine learning model 602.

As further shown in FIG. 20, in some examples at least some known inputs (obtained via the implanted accelerometer) comprise a rotational chest wall motion 1672, a breath-to-breath timing 1674, and/or a respiratory motion amplitude 1676. It will be understood that these inputs are mere examples, and that the known inputs (from the implanted accelerometer signal) may comprise any sensed physiologic information (including respiratory information) pertinent to determining an estimated blood oxygen desaturation. It will be understood that the respiration motion amplitude 1676 may comprise at least one aspect of rotational chest wall motion 1672.

By providing such known inputs (1671) and known outputs (1678) to the constructable data model 1677, a constructed data model 1683 (FIG. 21) may be obtained. As noted elsewhere, the constructable data model 1677 (FIG. 20) may comprise a trainable machine learning model and the constructed data model 1683 (FIG. 21) may comprise a trained machine learning model.

In some examples, just one or some of the inputs 1671 may be used, while all of the inputs 1671 may be used in some examples.

FIG. 21 is a diagram schematically representing an example method 1680 of using a constructed data model 1683 for determining estimated blood oxygen desaturation using internal measurements, such as via an implanted accelerometer. As shown in FIG. 21, currently sensed inputs 1681 are fed into the constructed data model 1683 (e.g. trained machine learning model), which then produces a determinable output 1688, such as a current estimated blood oxygen desaturation 1689, which is based on the current inputs 1681. In some examples, the current inputs 1681 are obtained via an implanted accelerometer (e.g. 285 in FIG. 2, 304A, 322A in FIGS. 2B-3B) and the current inputs 1681 (e.g. 1682, 1684, 1686) correspond to the types of known inputs 1671 (e.g. 1672, 1674, 1676 in FIG. 20) obtained via the implanted accelerometer.

In some examples, just one or some of the inputs 1681 may be used, while all of the inputs 1681 may be used in some examples.

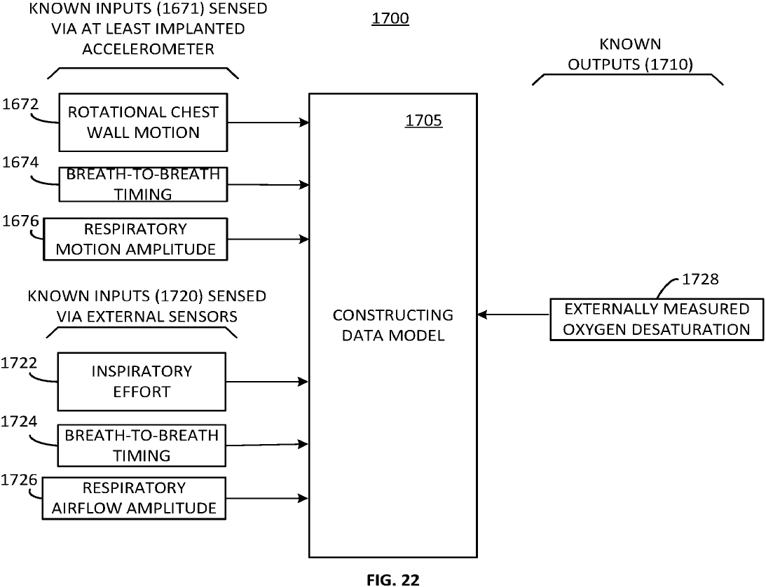

FIG. 22 is diagram schematically representing an example method 1700 of constructing a data model. Method 1700 may comprise at least some of substantially the same features and attributes as method 1670 (FIG. 20), except further comprising additional external known inputs 1720, e.g. inputs which are sensed via external sensors. In some examples, using both the internally measured known inputs 1671 (e.g. 1672, 1674, 1676) and the externally measured known inputs 1720 (e.g. 1722, 1724, 1726) may enhance accuracy, robustness, etc. in constructing the data model (1705). In some examples, the additional externally measured inputs 1720 may comprise inspiratory effort 1722, breath-to-breath timing 1724, and/or respiratory airflow 1726 (e.g. amplitude). It will be understood that additional and/or other externally measured inputs 1720 may be used which are pertinent to respiration, oxygen desaturation, and/or related parameters.

Accordingly, using both the internally measurable known inputs 1671 and the externally measurable known inputs 1720, and known outputs 1710 (such as externally measurable blood oxygen desaturation 1728), the data model can be constructed as shown at 1705 in FIG. 22.

In some examples relating to at least FIG. 22, just one or some of the inputs 1671 and just some of the inputs 1720 may be used, while all of the inputs 1671 and/or all of the inputs 1720 may be used in some examples.

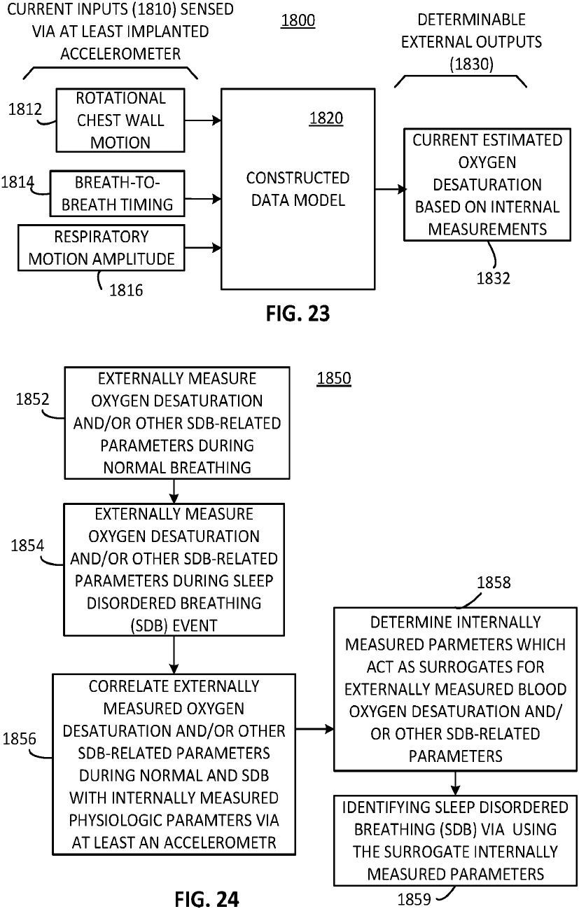

FIG. 23 is a diagram schematically representing an example method 1800 of using a constructed data model 1820 for determining estimated blood oxygen desaturation using internal measurements, such as via an implanted accelerometer. The constructed data model 1820 is obtained via the method 1700 in FIG. 22 via constructing data model at 1705, which includes the additional externally measurable known inputs 1720. As shown in FIG. 23, currently sensed inputs 1810 are fed into the constructed data model 1820 (e.g. a trained machine learning model), which then produces a determinable output 1830, such as a current estimated blood oxygen desaturation 1832, which is based on the current inputs 1810. In some examples, the current inputs 1810 are obtained via an implanted accelerometer (e.g. 285 in FIG. 2B, 304A, 322A in FIGS. 3A-3B) and the current inputs 1810 (e.g. 1812, 1814, 1816) correspond to the types of known inputs 1671 (e.g. 1672, 1674, 1676 in FIG. 20) obtained via the implanted accelerometer.

Accordingly, at least some of the various methods described in association with at least FIGS. 17A-23 may determine an internally measurable, estimated blood oxygen desaturation, which in turn, may be used to determine sleep disordered breathing and/or to determine other disease burden indicator(s). In some examples, the internally measurable, estimated blood oxygen desaturation may be used with additional internally measurable information to determine sleep disordered breathing and/or other disease burden indicators.

FIG. 24 is a flow diagram schematically representing an example method 1850 to determine which internally measured parameters may act as surrogates for externally measured blood oxygen desaturation and/or other sleep disordered breathing (SDB) parameters. As shown at 1852 in FIG. 24, method 1850 comprises externally measuring blood oxygen desaturation (and/or other SDB related parameters) during normal breathing (at 1852) and during sleep disordered breathing, as shown at 1854. At 1856, method 1850 comprises correlating the externally measured blood oxygen desaturation (during both normal and SDB) with internally measured physiologic parameters (sensed via at least accelerometer 285) during normal and sleep disordered breathing (SDB). At 1858, method 1850 comprises determining which internally measured physiologic parameters, alone or in combination, act as a surrogate for externally measured blood oxygen desaturation. The internally measurable parameters may be based on sensing via an implantable acceleration sensor and/or other internal sensing. As further shown at 1859 in FIG. 24, in some examples method 1850 further comprises identifying sleep disordered breathing (SDB) via an estimated blood oxygen desaturation based on the (surrogate) internally measured parameters.

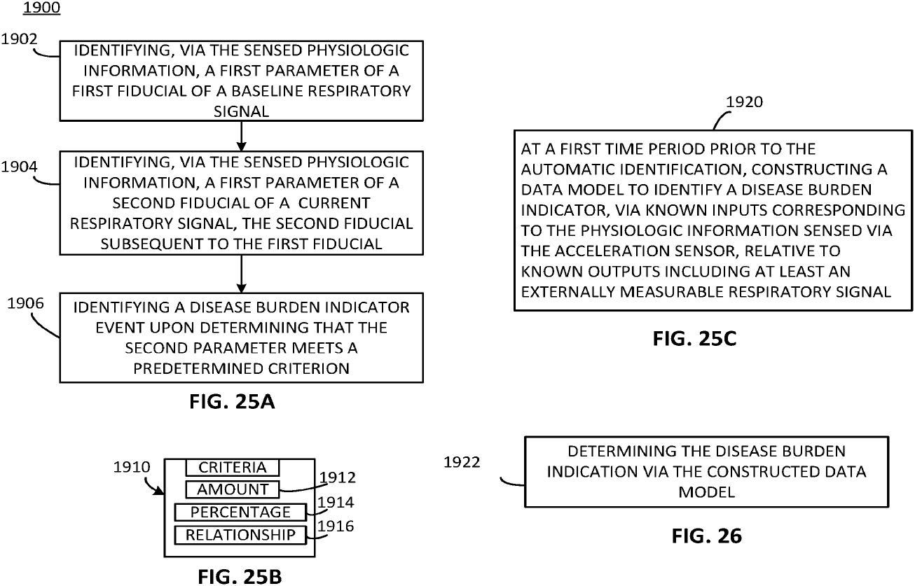

FIG. 25A is a flow diagram schematically representing an example method 1900 of identifying a sleep disordered breathing event. As shown at 1902, in some examples method 1900 comprises identifying, via the sensed physiologic information, a first parameter of a first fiducial of a baseline respiratory signal. In some such examples, the sensed physiologic information is obtained via an implanted accelerometer (e.g. 285 in FIG. 2B, 304A/322A in FIGS. 3A-3B). As shown at 1904, method 1900 comprises identifying, via the sensed physiologic information, a second parameter of a second fiducial of a current respiratory signal, wherein the second fiducial is subsequent to the first fiducial. As shown at 1906, method 8900 comprises identifying a sleep disordered breathing (SDB) event upon determining that the second parameter amplitude meets a predetermined criterion. In some examples, the predetermined criteria is met when the second parameter differs from the first parameter by a predetermined amount or the second parameter equals or exceeds the predetermined criteria when the predetermined criteria is an amount.

As shown in FIG. 25B, the criterion 1910 may comprise an amount 1912, a percentage 1914, and a relationship 1916. For example, the second amplitude may be an amount 1912 less than the first amplitude or greater than the first amplitude, depending on the particular fiducial of the respiratory signal being monitored. In another example the second amplitude may be a percentage less than the first amplitude or greater than the first amplitude, depending on the particular respiratory fiducial. In another example, the second amplitude may have a particular relationship (e.g. ratio, other) relative to the first amplitude. Each of the amount, percentage, or relationship may be selectable.

In some examples, the baseline and current respiration signal may comprise an internally measurable respiratory signal. In some such examples, this internally measurable signal may be obtained via an implanted acceleration sensor, such as via sensing rotational chest motion as previously described. In some examples, the internally measurable respiratory information may be used to provide an estimated respiratory airflow signal. In some examples, the internally measurable respiratory information may be used to identify sleep disordered breathing (SDB), whether in association with a data model or without such data models.

In some examples, with reference to the method 1900 in FIG. 25A, the internally measured respiratory signal comprises an estimated respiratory airflow signal or other surrogate for externally measured respiratory flow limitations. Moreover, in such examples, the first parameter comprises a first amplitude and the first fiducial comprises at least one first respiratory cycle, while the second parameter comprises a second amplitude and the second fiducial comprises a second respiratory cycle. Via this arrangement, the method may identify sleep disordered breathing (SDB) upon determining the second amplitude is less than the first amplitude. Via such an arrangement, one example implementation of the method 1900 may comprise identifying, via the sensed physiologic information, a first amplitude of at least one respiratory cycle (e.g. at least one breath) of a baseline estimated respiratory airflow signal. This example implementation also comprises identifying, via the sensed physiologic information, a second amplitude of a second respiratory cycle (e.g. a second breath) of a current estimated respiratory airflow signal, wherein the second respiratory cycle is subsequent to the at least one first respiratory cycle. Moreover, this example implementation further comprises identifying a sleep disordered breathing (SDB) event upon determining that the second amplitude meets a criteria relative to the first amplitude.

In some examples, the second respiratory cycle in method 1900 may comprise a sleep disordered breathing event (e.g. an apnea) followed by a recovery period. In this context, a recovery period is marked by airway patency following an interval of obstruction. Due to the preceding cessation of respiration, a recovery period following apnea is generally marked by a large amplitude (as compared to unobstructed sleeping baseline) breath(s) and sometimes referred to as “rescue breath(s)”. In some examples, the recovery period may be identified by high signal amplitude, and/or a rapid increase in tidal volume, and/or a spike in respiration rate.

In some examples, the characteristic features of an apnea (e.g. cessation of inspiration) followed by a recovery period may be spread over several respiratory cycles, such as when the inspiratory period exhibits a low amplitude (compared to a normal breath) and when the recovery period may be less pronounced than a typical recovery period following an apnea. In some such examples, the recovery period may be identified by a slight increase in signal amplitude, and/or a moderate increase in tidal volume, and/or an abnormal increase in respiration rate.

In some such examples in which an apnea and recovery period are spread over several respiratory cycles, the increased signal amplitude (compared to the baseline respiratory signal) identified in such several respiratory cycles may be combined together to produce an aggregate-type sleep disordered breathing (SDB) event. In one aspect, an identified aggregate-type of SDB event may warrant counting as a SDB event even though the breathing behavior does not meet the primary criteria for a SDB event in a single respiratory cycle, such an obstructive sleep apnea (OSA). Moreover, such an identification of an aggregate-type sleep disordered breathing (SDB) event also may be used to invoke treatment via stimulation of an upper-airway-patency nerve. Such examples may be useful in identifying sleep disordered breathing (SDB) behavior, which may sometimes be referred to as several slow obstructive cycles in which a patient comes in and out of breathing.

In some such examples of identifying aggregate-type SDB, the increased signal amplitude observed over several respiratory cycles may sometimes be referred to as an increased envelope of signal amplitude over several second respiratory cycles. In some examples, the signal amplitude envelope may be generated using filtering (e.g. mean filter, median filter, or a low pass filter with corner below a baseline respiration rate). Such a signal amplitude envelope may capture lower frequency (versus physiologic respiration rate) shifts in amplitude over time in a signal correlated with the mechanical energy of respiration. In such examples, this arrangement may correspond to the second fiducial comprising a series of second respiratory cycles, and the first parameter of the second fiducial comprising a second signal amplitude envelope aggregated over the series of second respiratory cycles. The second signal amplitude envelope may comprise a sum of the amplitude of the current respiratory signal for the series of respiratory cycles.

In some such examples regarding an aggregate-type SDB, a baseline respiratory signal may comprise a baseline signal amplitude envelope. In some examples, the baseline signal amplitude envelope may be determined with regard to a particular frequency range within the baseline respiratory signal and/or the increased signal amplitude envelope may be determined with regard to the same particular frequency range. In such examples, this arrangement may correspond to the first parameter of the first fiducial (of a baseline respiratory signal) comprising a first signal amplitude envelope within a first frequency range, and wherein the first signal amplitude envelope comprises an amplitude of the baseline respiratory signal for the normal respiratory cycle.

In some instances, the baseline respiratory signal may sometimes be referred to as a historical respiratory signal sensed for/of a patient at earlier point in time than the current respiratory signal. In some examples, the at least one first respiratory cycle (e.g. at least one first breath) comprises the respiratory cycle immediately prior to the second respiratory cycle (e.g. second breath). However, in some examples the second respiratory cycle (subsequent breath) may be more than one respiratory cycle (e.g. one breath) later than the at least one respiratory cycle (e.g. first breath) of the baseline respiratory signal. In some such examples, the at least one first respiratory cycle comprises some multiple number of respiratory cycles (e.g. 3, 4, 5, etc.).

In some examples, the method 1900 may be implemented based on a data model. Accordingly, as shown at 1920 in FIG. 25C, method 1900 may further comprise, at a time period prior to the identification the first amplitude of the current respiratory signal, constructing a data model to identify sleep disordered breathing via known inputs corresponding to the physiologic information sensed via the acceleration sensor, relative to known outputs, such as but not limited to, an externally measurable respiratory signal. In some such examples, the known inputs may comprise at least respiratory information including but not limited to the baseline respiratory signal. In some such examples, the externally measurable respiratory signal may comprise a respiratory airflow signal, such as but not limited to a nasal airflow signal.

Upon constructing the data model at 1920, method 1900 may further comprise identifying the sleep disordered breathing (e.g. at 1906 in FIG. 25A) via the constructed data model, as shown at 1922 in FIG. 26.

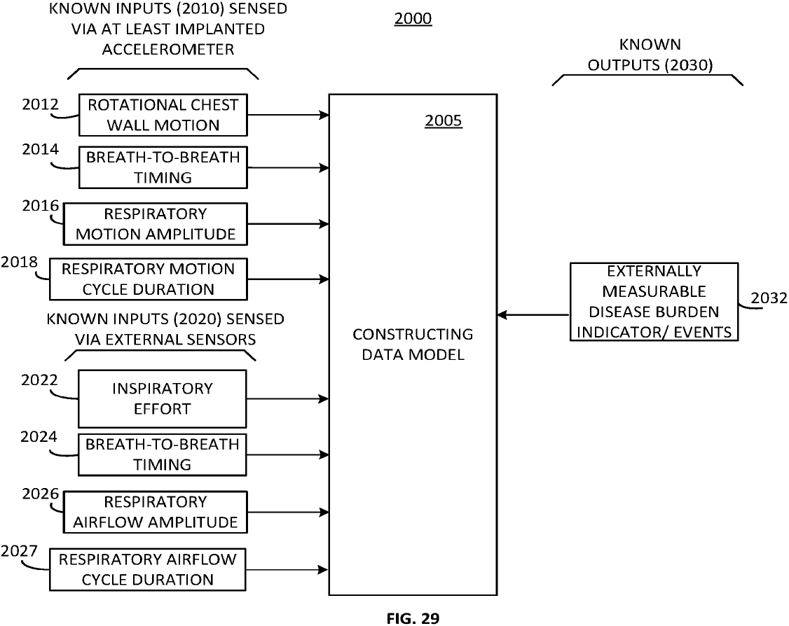

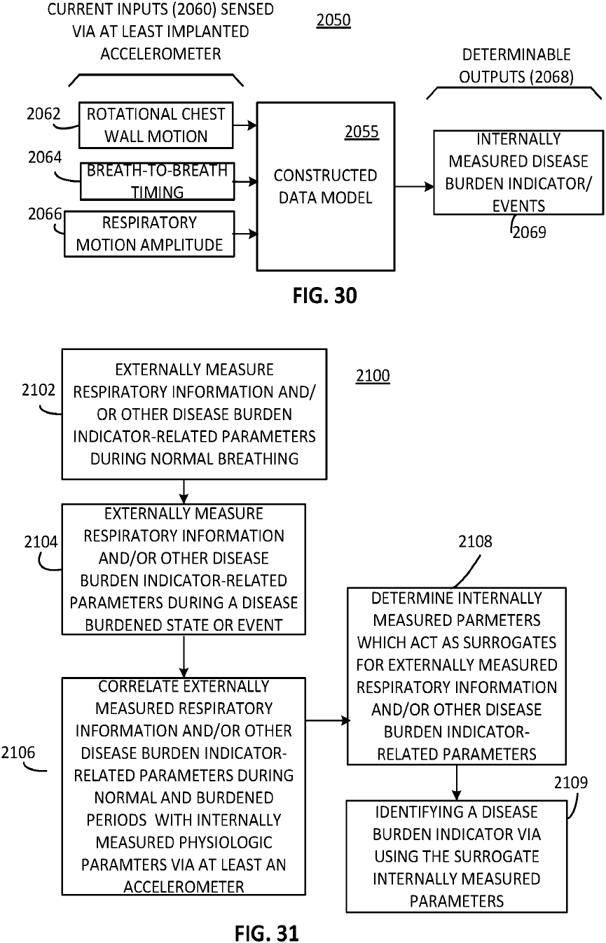

At least some details regarding constructing the data model (FIG. 25C) are further described later in association with at least FIGS. 29-30.

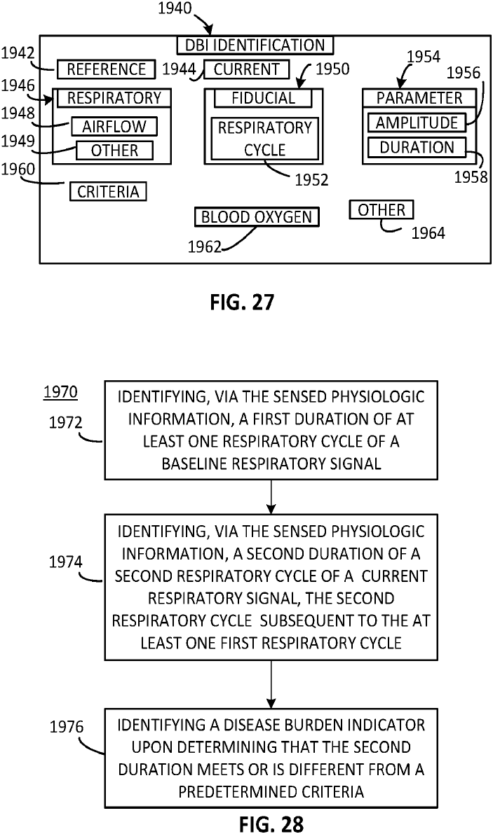

FIG. 27 is a block diagram schematically representing an example sleep disordered breathing (SDB) identification engine 1940. In some examples, at least some aspects of method 1900 as described in association with FIGS. 25A-25C, 26 may be implemented via engine 1940. Moreover, engine 1940 may implement further aspects of method 1900 and/or other methods as later described in association with at least FIGS. 28-29.

As shown in FIG. 27, in some examples engine 1940 comprises reference element 1942 to identify and/or track a reference physiologic signal, such as a baseline physiologic signal and comprises a current element 1944 to identify and/or track a current physiologic signal. In some examples, the physiologic signal comprises a respiratory signal, which in some examples may be obtained via an acceleration sensor (e.g. 285 in FIG. 2B).

As further shown in FIG. 27, in some examples engine 1940 comprises a respiratory signal element 1946, which may comprise an estimated respiratory airflow signal element 1948 and other element 1949 in some examples. The estimated respiratory airflow signal comprises internally measurable physiologic information, obtained via at least respiratory motion sensed via an acceleration sensor, which is correlated to an externally measurable respiratory airflow signal, with the estimated respiratory airflow signal being used to identify sleep disordered breathing, such as in FIG. 25C. In some examples, the estimated respiratory airflow signal element 1948 may correspond to, and/or be correlated relative to, an estimated flow limitation signal and/or an estimated inspiratory effort signal. The other element 1949 corresponds to other estimated or actual internally, physiologically sensed information/sources to which externally measurable respiratory signal (or other externally measureable signal) may be correlated, and which is indicative of sleep disordered breathing.

In some examples, via fiducial element 1950 and parameter element 1954, a parameter of a fiducial of the measured signal is identified and tracked to identify sleep disordered breathing (SDB). In some examples, per element 1952, the fiducial may comprise a respiratory cycle, portions of a respiratory cycle (e.g. inspiration, active expiration, expiratory pause), offsets and/or onsets of portions of a respiratory cycles, peaks and/or valleys of portions of a respiratory cycle, etc. In some examples, per parameter element 1954, the parameter may comprise an amplitude 1956, a duration 1958, and/or other parameter of a fiducial, such as a respiratory cycle, portion of a respiratory cycle, etc.

In some examples, via the criteria parameter 1960, engine 1940 may evaluate differences between a reference signal (1942) and a current signal (1944). As previously described in association with at least FIG. 25B, the criteria 1910 may comprise an amount, a percentage, a relative comparison (e.g. ratio) between a parameter of the current signal relative to the baseline signal (or vice versa).

In some examples, the SDB identification engine 1940 may comprise a blood oxygen parameter 1962 to use an estimated blood oxygen desaturation signal, in addition to the respiratory signal or instead of the respiratory signal, to identify sleep disordered breathing (SDB). In some examples, per parameter 1964, the SDB identification engine 1940 may utilize physiologic information other than respiratory and/or blood oxygen information to identify sleep disordered breathing (SDB).

With further reference to at least the parameter element 1954 in FIG. 27, it will be understood that the term “element” does not connote a mechanical or physical element but rather a parameter or aspect of engine 1940 and/or operating the engine 1940.

In some examples, with further reference to the method 1900 in FIG. 25A, the respiratory signal comprises an estimated respiratory airflow signal, the first parameter comprises a first duration and the first fiducial comprises at least one first respiratory cycle, and the second parameter comprises a second duration and the second fiducial comprises a second respiratory cycle, and a SDB event may be identified upon determining the second duration is greater than a predetermined criteria. In some examples, the respiratory signal may comprise an internally measurable respiration signal or information which is indicative of respiratory flow limitations associated with sleep disordered breathing (SDB) without necessarily being an estimated respiratory airflow signal.

Via such an arrangement, one example implementation of method 1900 may comprise an example method 1970, as shown at 1972 in FIG. 28, comprising identifying, via the sensed physiologic information, a first duration of at least one respiratory cycle (e.g. at least one breath) of a baseline respiratory signal, such as but not limited to a baseline estimated respiratory airflow signal. As shown at 1974, method 1970 comprises identifying, via the sensed physiologic information, a second duration of a second respiratory cycle (e.g. a second breath) of a current respiratory signal (e.g. current estimated respiratory airflow signal), wherein the second respiratory cycle is subsequent to the at least one first respiratory cycle. As shown at 1976, method 1970 comprises identifying a disease burden indicator (e.g. sleep disordered breathing (SDB) event, other) upon determining that the second duration meets a predetermined criteria, such as but not limited to an amount, a percentage, and/or a comparison to the first duration. In some examples, the determination of a disease burden indicator may comprise determining that the second duration is less than the predetermined criteria while in some examples, the determination of a disease burden indicator may comprise determining that the second duration is greater than the predetermined criteria

In some examples, the example method 1970 may be performed instead of the above-described example implementations of method 1900 in which the first and second parameters comprise first and second amplitudes (as described above) and the first and second fiducials comprise first and second respiratory cycles. However, in some examples, the example method 1970 may be performed in addition to the above-described example implementations of method 1900 in which the first and second parameters comprise first and second amplitudes (as described above) and the first and second fiducials comprise first and second respiratory cycles.

However, in some examples, the example implementation of methods 1630, 1650 in FIGS. 17A, 18 regarding blood oxygen desaturation may be performed in addition to the above-described example implementations of method 1900 (FIG. 25A) in which the first and second parameters comprise first and second amplitudes (as described above) and the first and second fiducials comprise first and second respiratory cycles. In some examples, the example implementation of methods 1630, 1650 in FIGS. 17A, 18 regarding blood oxygen desaturation may be performed in addition to the above-described example implementations of method 1900 (and/or in method 1970 in FIG. 28) in which the first and second parameters comprise first and second durations and the first and second fiducials comprise first and second respiratory cycles.

In some examples, an example method (such as method 1900 in FIG. 25A) may identify an apnea (as SDB) upon sensing: (A) a decrease in peak signal excursion in a current estimated respiratory airflow signal by at least 90 percent relative to a baseline estimated respiratory airflow signal; and (B) a duration of the at least 90 percent decrease occurring for at least 10 seconds. In some instances, the latter criteria B may sometimes be expressed as sensing a duration of at least 10 seconds between “normal” breaths, where a normal breath comprises generally stable breathing (e.g. non-apnea breath and/or non-hypopnea breath).

In some such examples, the identified apnea may be deemed to be an obstructive sleep apnea (OSA) event when criteria A and B are met, and in addition, the method senses continued or increased inspiratory effort throughout the entire period of substantially absent airflow (e.g. a decrease in airflow by at least 90 percent). In some examples, the increased inspiratory effort may be internally measured via an implanted acceleration sensor (e.g. 285 in FIG. 2B, 304A, 322A in FIGS. 3A-3B, etc.) as an aspect of rotational chest motion or other acceleration-based sensing.

In some such examples, the identified apnea may be deemed to be a central sleep apnea (CSA) event when criteria A and B met, and in addition, upon the method sensing an absence of inspiratory effort throughout entire period of absent airflow (e.g. a decrease in estimated airflow by at least 90 percent).

In some examples, the identified apnea may be deemed a multi-type (or “mixed”) apnea when criteria A and B are met, and in addition, upon the method sensing absent inspiratory effort in the initial portion of the period of absent airflow, followed by resumption of inspiratory effort in the second portion of the period of absent airflow. In some examples, the multiple type sleep apnea may be identified according to at least some of substantially the same features and attributes as described in U.S. Pat. Application “MULTIPLE TYPE SLEEP APNEA” published as U.S. 2020/0147376 on May 14, 2020, and which is herein incorporated by reference.

In some examples, a respiratory event may be identified as a hypopnea upon sensing: (A) a decrease in peak signal of at least 30% in a current estimated respiratory airflow signal relative to a baseline estimated respiratory airflow signal; (B) the duration of the at least 30% decrease (in the estimated respiratory airflow signal) is at least 10 seconds; and (C) at least 3% change in blood oxygen desaturation (in a current estimated blood oxygen desaturation signal) relative to a baseline estimated blood oxygen desaturation signal. In some examples, the estimated blood oxygen desaturation signal is implemented according to at least some aspects of the methods described in association with at least FIGS. 17A-24. In some such examples, in order to score the event as a hypopnea, the event also is associated with an arousal. In some such examples, the arousal may comprise at least a neurological arousal. In some examples, the arousal may be identified as described in association with at least FIGS. 32A-34.

In some examples, in order to identify a hypopnea, criteria A and B are sensed and criteria C comprises at least 4% change in the current estimated blood oxygen desaturation relative to a baseline estimated blood oxygen desaturation signal.

In some examples, a hypopnea may be identified upon sensing a 50 percent reduction in estimated respiratory airflow (from comparing the current signal relative to a baseline signal) and at least 3 percent change in estimated blood oxygen desaturation (from comparing a current signal to a baseline signal). In some examples, a hypopnea may be identified upon sensing a 30 percent reduction in estimated respiratory airflow (from comparing the current signal relative to a baseline signal) and at least 4 percent change in estimated blood oxygen desaturation (from comparing the current signal relative to a baseline signal). In some such examples, instead of using an estimated respiratory airflow signal, the method may utilize other internally measurable respiratory information indicative of a respiratory flow limitation associated with sleep disordered breathing (SDB).

In some examples, the constructing of a data model in association with at least FIGS. 25C, 26 may be implemented according to an example method 2000 in FIG. 29. As shown in FIG. 29, known inputs 2010 sensed via at least an implanted accelerometer are provided to form a constructable data model 2005 and a known output 2030 is provided to the constructable data model 2005. The known output 2030 may comprise externally measurable disease burden indicator, which in some examples may comprise sleep disordered breathing (SDB) events, such as an obstructive sleep apnea, hypopnea, etc., which may be tracked via an apnea-hypopnea index (AHI) and/or other measures. In some examples, the externally measurable disease burden indicator (e.g. SDB events) may be identified via at least some of the externally measured parameters and/or information as previously described in association with at least FIGS. 13A-16B, etc. At least one of these externally measurable parameters comprises an externally measurable respiratory airflow signal. As previously described in association with at least FIGS. 9-11A, constructing the data model may comprise training a data model, such as one of the data models in data model types 600 in FIG. 10A with one of the example data model types comprising a machine learning model 602.

As further shown in FIG. 29, in some examples at least some known inputs 2010 (obtained via the implanted accelerometer) comprise a rotational chest wall motion 2012, a breath-to-breath timing 2014, a respiratory amplitude 2016, and/or a respiratory cycle duration 2018. In some such examples, the breath-to-breath timing 2014, respiratory amplitude 2016 and/or respiratory cycle duration 2018 are based on sensing respiratory motion, such as but not limited to the rotational chest wall motion 2012. It will be understood that these inputs are mere examples, and that the known inputs (from the implanted accelerometer signal) may comprise any sensed physiologic information (including respiratory information) pertinent to determine a disease burden indicator, such as sleep disordered breathing. As previously described in association with at least FIGS. 2B-3C, in some examples the known inputs may be obtained via sensing at least rotational chest wall motion via the implanted accelerometer 285 (FIG. 2).

By providing such known inputs (2010) and known outputs (2030) to the constructable data model 2005, construction of data model 2055 (FIG. 30) may be performed. As noted elsewhere, the constructable data model 2005 may comprise a trainable machine learning model and the constructed data model 2055 may comprise a trained machine learning model.

In some examples, construction of data model 2005 in FIG. 29 may comprise also using externally measured known inputs 2020 (e.g. 2022, 2024, 2026, 2027), such as externally measured respiratory effort 2022 (e.g. inspiratory effort), breath-to-breath timing 2024, respiratory airflow amplitude 2026, and respiratory cycle duration 2027 (such as from an respiratory airflow signal).

In some examples, using both the internally measured known inputs 2010 (e.g. 2012, 2014, 2016, 2018) and the externally measured known inputs 2020 (e.g. 2022, 2024, 2026, 2027) may enhance accuracy, robustness, etc. in constructing the data model (at 2005). It will be understood that additional and/or other externally measured inputs 2020 may be used which are pertinent to respiration, blood oxygen desaturation, and/or related parameters.

In some examples, just one or some of the inputs 2010 and just some of the inputs 2020 may be used, while all of the inputs 2010 and/or all of the inputs 2020 may be used in some examples.

Accordingly, using both the internally measurable known inputs 2010 and the externally measurable known inputs 2020, and known outputs 2030 (such as externally identifiable SDB events 2032, flow limitations, and the like), the data model can be constructed as shown at 2005 in FIG. 29.

FIG. 30 is a diagram schematically representing an example method 2050 of using a constructed data model for identifying sleep disordered breathing (SDB) according to a respiratory signal as previously described in association with at least FIGS. 25A-29 and/or other examples herein. As shown in FIG. 30, currently sensed inputs 2060 are fed into the constructed data model 2055 (e.g. trained machine learning model), which then produces a determinable output 2068, such as internally measured disease burden indication 2069, which is based on the current inputs 2060. In some examples, the disease burden indicator 2069 may comprise sleep disordered breathing. In some examples, the current inputs 2060 are obtained via an implanted accelerometer (e.g. 285 in FIG. 2B, 304A/322A in FIGS. 3A-3B) and the current inputs 2060 (e.g. 2062, 2064, 2066) correspond to at least the types of known inputs 2010 (e.g. 2012, 2014, 2016 in FIG. 29) obtained via the implanted accelerometer.

Accordingly, in some examples, the methods and/or arrangements described in association with at least FIGS. 29-30 may be used to implement the previously described methods 1900 (FIGS. 25A-26), 1940 (FIG. 27) and/or 1970 (FIG. 28).

FIG. 31 is a flow diagram schematically representing an example method 2100 to determine which internally measured parameters may act as surrogates (for externally measurable parameters) to identify a disease burden indicator, which in some examples may comprise sleep disordered breathing (SDB) events. As shown at 2102 and 2014 in FIG. 31, method 2100 comprises externally measuring respiratory information during normal breathing and during a period in which the patient is experiencing a disease burdened state or event, which in some examples may comprise a sleep disordered breathing event. At 2106, method 2100 comprises correlating the externally measured respiratory information (during both normal and a disease burdened state/event) with internally measured physiologic parameters (sensed via at least accelerometer 285) during normal and during the disease burdened state/event (e.g. sleep disordered breathing, other). At 2108, method 2100 comprises determining which internally measured physiologic parameters (e.g. which respiratory parameters), alone or in combination, act as a surrogate for externally measured respiration and/or to identify a disease burdened indicator (e.g. sleep disordered breathing (SDB), other). Finally, at 2109 in FIG. 31, method 2100 comprises identifying a disease burden indicator (e.g. sleep disordered breathing (SDB), other) using the identified “surrogate” internally measured physiologic parameters.

In some examples, patients experiencing a disease burdened state (e.g. sleep disordered breathing (SDB) such as obstructive sleep apneas) typically experience arousals, such as a neurological arousal (i.e. microarousal). A neurological arousal comprises a change in brain waves for a minimum duration, and may be measured via an electroencephalography (EEG), as further described later. In some instances, the neurological arousal arising from sleep disordered breathing (or other diseases) may be accompanied by non-neurological physiologic behavior.

In some examples, at least some of this non-neurological physiologic behavior may be sensed via an internal sensor, such as but not limited to, an implantable accelerometer (e.g. 285 in FIG. 2B, 304A, 322A in FIGS. 3A-3B). In some such examples, this internally sensed physiologic information may be used to identify an arousal, which may include identifying an internally estimated arousal in some examples. The internally estimated arousal may be performed in the absence of external sensing (e.g. EEG, other) for neurological arousals and/or in the absence of external sensing of other physical manifestations of such neurological arousals.

With this in mind FIGS. 32A-34 provide several example methods and arrangements for identifying an arousal (e.g. associated with sleep disordered breathing) using internally measurable physiologic information, such as obtained via an implantable accelerometer.

FIG. 32A is a diagram schematically representing an example method 2150 of identifying an arousal. In some examples, the identification of an arousal may be used as part of determining a disease burden indicator (such as but not limited to sleep disordered breathing (SDB)) via the methods, engines, arrangements, etc. described in association with at least FIGS. 1-31 and FIGS. 35-102. As shown at 2152 in FIG. 32A, method 2150 comprises identifying, via internally sensed physiologic information, a first value of a first arousal-related parameter. At 2154, method 2150 comprises identifying, via the sensed physiologic information, a second value of the first arousal-related parameter, and at 2156, method further comprises identifying an arousal upon determining that the second value differs from the first value by a predetermined criteria. In some examples, the internally sensed physiologic information may comprise information sensed via an implanted acceleration sensor (e.g. 285 in FIG. 2B, 304A, 322A in FIGS. 3A-3B, etc.) in some examples.

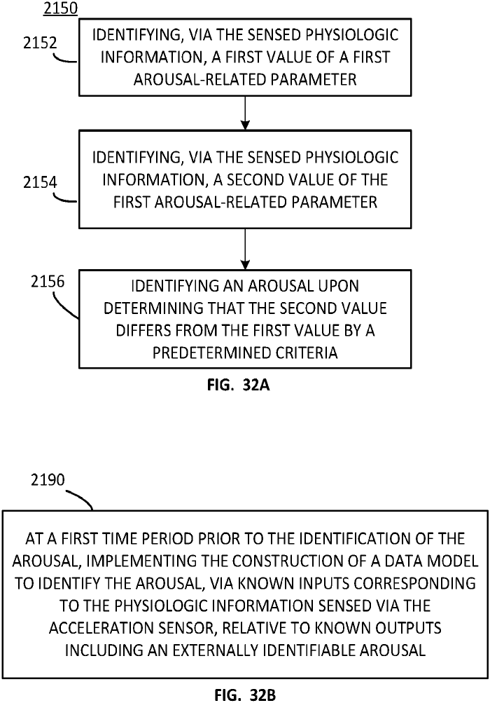

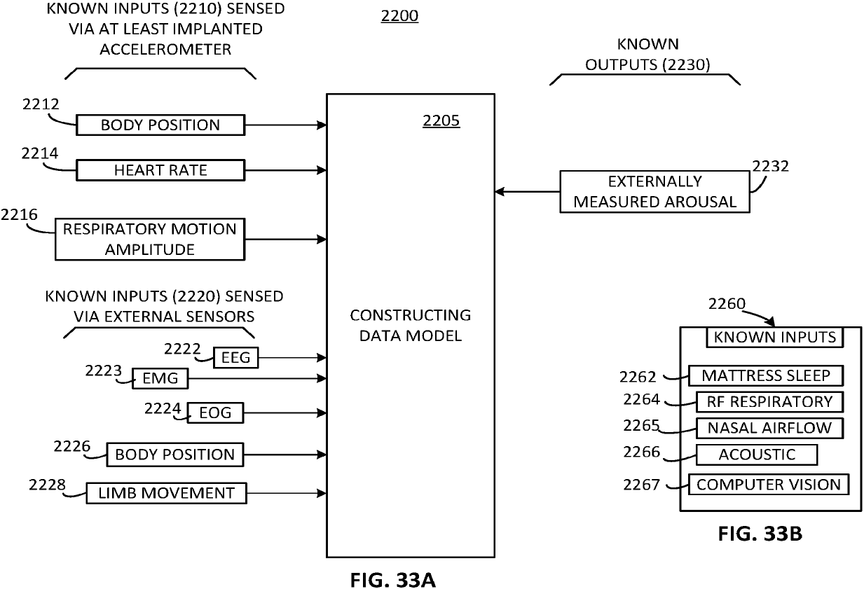

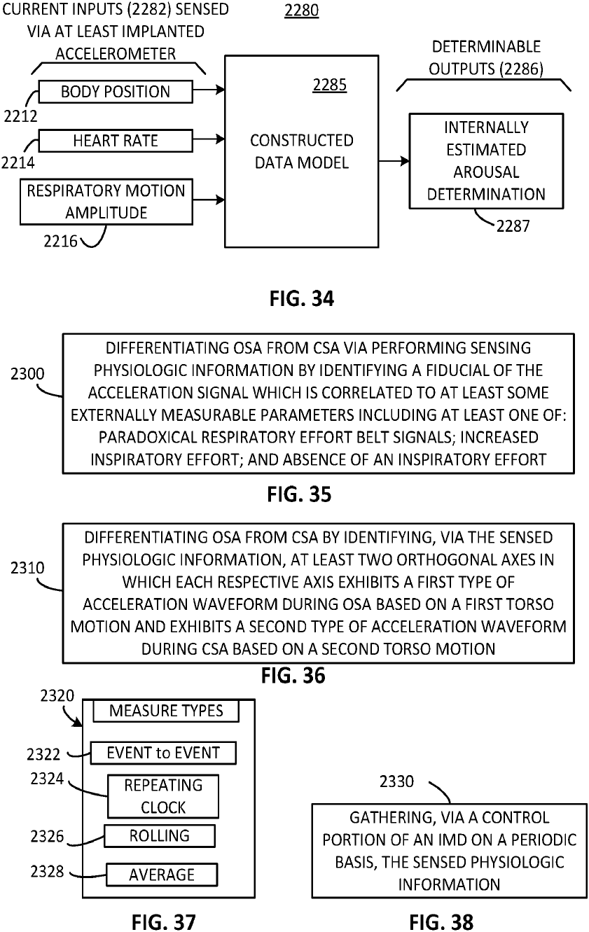

With further reference to method 2150 in FIG. 32A and as also shown later in FIGS. 33A, 34, in some examples the first arousal-related parameter may comprise at least one of a respiratory motion signal, a gross body movement signal (e.g. body position), and a heart rate signal.

In some examples in which the first arousal-related parameter comprises a body movement signal, method 2150 in FIG. 32A may compare the respective first and second values of the body movement signal according to at least one of a signal amplitude, an integral of the signal amplitude, a square of the signal amplitude, and an integral of the square of the signal amplitude associated with the body movement signal. In some such examples, the body movement signal may comprise posture information, such as a change in posture. However, in some examples, the body movement signal omits posture information.

In some examples in which the first arousal-related parameter comprises respiratory motion (e.g. chest wall movement), method 2150 in FIG. 32A may compare the respective first and second values of an amplitude in a frequency band corresponding to apneic movement. In some examples, the frequency band may comprise a respiration frequency band. In some such examples, this frequency band may comprise an empirically determined signal frequency (such as via bandpass filtering or a fast Fourier transform (FFT)) that is correlated with sleep apnea events.

In some examples, the sensed changes (e.g. an increase or decrease) in chest wall movement may be due to increased respiratory effort or changes in motion shape due to the changes in motion (resulting in different harmonic content or other non-linear behavior).

In some examples, the values and/or fiducials of the sensed respiratory motion may comprise at least one of: (A) a standard deviation of an amplitude of a respiratory motion signal, wherein the difference between the second value and the first value comprises an increase in the standard deviation; and (B) a signal-to-noise ratio in respiration signal, wherein the difference between the second value and the first value comprises a decrease.

In some examples in which the first arousal-related parameter comprise heart rate information, the heart rate information may comprise heart rate variability (HRV) by which an arousal event may be identified upon determining that the second value of the heart rate variability (HRV) is greater than the first value of the heart rate variability (HRV) by at least a predetermined criteria.

In some examples, the first arousal-related parameter also may comprise breath-to-breath timing (e.g. respiratory variability) and/or estimated tidal volume based on an amplitude of the acceleration sensor.

In a manner similar to some previously described examples relating to disease burden indication generally, estimated blood oxygen desaturation and/or estimated respiratory airflow signals, a method of identifying arousals using internally sensed physiologic information may be enhanced via employing a data model, such as but not limited to a machine learning model (MLM) or similar techniques.

Accordingly, as shown at 2190 in FIG. 32B, one example method comprises, at a first time period prior to the identification of the arousal, implementing the construction of a data model to identify the arousal, via known inputs corresponding to the physiologic information internally sensed via at least the implantable acceleration sensor, relative to known outputs including an externally identifiable arousal.

FIG. 33A is diagram schematically representing an example method 2200 of constructing a data model for use in identifying an arousal using internally measurable physiologic information. In some examples, the method 2200 comprises one example implementation of constructing data model 2190 in FIG. 32B and/or to implement method 2150 in FIG. 32A. As shown in FIG. 33A, known inputs 2210 sensed via at least an implanted accelerometer are provided to form a constructable data model 2205 and a known output 2230 is provided to the constructable data model 2205. The known output 2230 may comprise externally measurable arousals, which may be neurological, physical, and/or both. In some examples, the externally measurable arousals may be identified via at least some of the externally measured parameters and/or information as previously described in association with at least FIGS. 13A-16B, parameters 2260 in FIG. 33B, and the like. As previously described in association with at least FIGS. 8-12B, constructing the data model may comprise training a data model, such as one of the data models in data model types 600 in FIG. 10A with one of the example data model types comprising a machine learning model 602.

As further shown in FIG. 33A, in some examples at least some known inputs 2210 (obtained via at least the implanted accelerometer) comprise a body position 2212, a heart rate 2214, and/or a respiratory motion amplitude 2216. In some examples, just one or some of these inputs 2210 may be used, while all the inputs 2210 may be used in some examples. It will be understood that these inputs are mere examples, and that the known inputs (from the implanted accelerometer signal) may comprise any sensed physiologic information (including respiratory information) pertinent to determining an arousal, whether neurological, physical, or both. As previously described in association with at least FIGS. 3A-3C, at least some of the known inputs may be obtained via sensing at least rotational chest wall motion via the implanted accelerometer (e.g. at least 285 in FIG. 2B, 304A/322A in FIGS. 3A-3B).

By providing such known inputs (2210) and known outputs (2230) to the constructable data model 2205, a constructed data model 2285 (FIG. 34) may be implemented. As noted elsewhere, the constructable data model 2285 may comprise a trainable machine learning model and the constructed data model 2285 may comprise a trained machine learning model.

In some examples, construction of data model 2205 may comprise also using externally measured known inputs 2220, such as but not limited to EEG 2222, EMG 2223, EOG 2224, body position 2226, and/or limb movement 2228, as previously noted in association with at least FIG. 16A.

In some such examples, the EEG parameter 2222 may be used to identify and/or track a neurological arousal upon the patient being asleep in one of the sleep stages (e.g. N1, N2, N3, or in REM) and if there is an abrupt shift of EEG frequency (including alpha, theta and/or frequencies greater than 16 Hz (but not spindles) that lasts at least 3 seconds, with at least 10 seconds of stable sleep preceding the change. In some examples, identifying an arousal during REM sleep also demands a concurrent increase in submental EMG lasting at least 1 second.

In some examples, using both the internally measured known inputs 2210 (e.g. 2212, 2214, 2216) and the externally measured known inputs 2220 (e.g. 2222, 2223, 2224, 2226, 2228) may enhance accuracy, robustness, etc. in constructing the data model (at 2205). It will be understood that additional and/or other externally measured inputs 2220 may be used which are pertinent to identifying arousals and/or related parameters.

Accordingly, using both the internally measurable known inputs 2210 and the externally measurable known inputs 2220, and known outputs 2230 (such as externally identifiable arousals 2232), the data model can be constructed as shown at 2205 in FIG. 33A.

In some examples, method 2200 may comprise providing known inputs (to construct a data model) in addition to, or instead of, the known inputs 2220 in FIG. 33A. For instance, in some examples one or more of the known inputs 2260 in FIG. 33B may be provided to the data model 2205 in FIG. 33A when constructing (FIG. 33A) the constructed data model 2285 (FIG. 34).

With this in mind, FIG. 33B is a diagram schematically representing example known inputs 2260 for constructing a data model, which may be used as known inputs in method 2200 of FIG. 33A. The externally measurable known inputs 2260 may be used instead of, or in addition to, known inputs 2220 in FIG. 33A. In some such examples, just one or some of the inputs 2220 may be used, while all the inputs 2220 may be used in some examples. Similarly, in some examples, just one or some of these inputs 2260 may be used, while all the inputs 2260 may be used in some examples. Of course, just some of the external inputs 2220 may be mixed in various combinations with just some of the external inputs 2260.

In some examples, the externally measurable known inputs 2260 shown in FIG. 33B may comprise mattress sleep data 2262 (e.g. tracking patient movements, sounds, static position, etc. during sleep) radiofrequency-detectable (RF) respiratory information 2264, nasal airflow 2265 (e.g. respiratory airflow), acoustic microphone 2266 (e.g. snoring, breathing sounds), and/or computer vision 2267 to observe the patient during sleep. In some examples, the computer vision input may be obtained via a computer vision system may comprise single camera, stereo camera, and/or projected light. In some examples, the acoustic parameter 2266 may comprise at least one of a bedside monitor and a smartphone to externally record noises during a sleep period/treatment period. Such recorded acoustic information may be used to compare the externally recorded noises with the identified arousal events to at least partially determine presence of at least some identified arousal events which are false negative identifications.

In a manner similar to method 2200, method 2250 comprises providing such known inputs (2210) and known inputs (2260 and/or 2220) to the constructable data model 2205 to implement a constructed data model 2285 as shown in FIG. 34. As noted elsewhere, the constructable data model 2205 may comprise a trainable machine learning model and the constructed data model 2285 may comprise a trained machine learning model.

FIG. 34 is a diagram schematically representing an example method 2280 of using a constructed data model 2285 for performing an estimated arousal determination, such as via an implanted accelerometer. As shown in FIG. 34, currently sensed inputs 2282 are fed into the constructed data model 2285 (e.g. trained machine learning model), which then produces a determinable output 2286, such as an internally estimated arousal 2287, which is based on the current inputs 2282. In some examples, the current inputs 2282 are obtained via at least an implanted accelerometer (e.g. 285 in FIG. 2, 304A/322A in FIGS. 3A-3B) and the current inputs 2282 (e.g. 2212, 2214, 2216) correspond to at least the types of known inputs 2210 (e.g. 2212, 2214, 2216 in FIG. 33A) obtained via at least the implanted accelerometer.

Accordingly, the methods and/or arrangements described in association with at least FIGS. 33A-34 may be used to internally sensed signals, such as via at least an implanted accelerometer (e.g. 285 in FIG. 2, 304A/322A in FIGS. 3A-3B), to determine an estimated arousal based on the internally sensed signals.

This estimated arousal determination (2287 in FIG. 34) may be used to assess, track, etc. sleep quality, and in some instances, may be used to identify a disease burden indicator, which may in some examples comprise evaluating sleep disordered breathing and/or at least partially identifying sleep disordered breathing (SDB), in some examples. Moreover, depending on which of the particular inputs (from among inputs 2210, 2220, 2260) are employed to construct a data model (e.g. 2205, 2285), the estimated arousal determination (2287) may comprise an estimated neurological arousal determination, an estimated physical arousal determination, or both.

In some examples, such as in particular classes of patients (e.g. children) an arousal may comprise a respiratory-related arousal (RERA). This information also may be used to identify a disease burden indication. In some examples, the particular patient behavior involving a respiratory-related arousal (RERA) does not qualify as sleep disordered breathing (SDB) defined as an apnea or a hypopnea. In some such examples, if electing to score respiratory effort-related arousals, certain types of respiratory behavior may be scored as a respiratory effort-related arousal (RERA) if there is a sequence of breaths lasting ≥10 seconds characterized by increasing respiratory effort or by flattening of the inspiratory portion of the nasal pressure (diagnostic study) or PAP device flow (titration study) waveform leading to arousal from sleep when the sequence of breaths does not meet criteria for an apnea or hypopnea.

With this in mind, in some examples the methods and/or devices described above in association with at least FIG. 32A-34 to identify arousals may be applied to identify a respiratory-related arousal (RERA).

In some examples, assuming one of the previously described examples is used to identify a disease burden indication, such as sleep disordered breathing (SDB), further information may be desired regarding the sleep disordered breathing. As shown in FIG. 35, method 2300 comprises differentiating obstructive sleep apnea (OSA) from central sleep apnea (CSA) via performing sensing physiologic information by identifying a fiducial of the acceleration signal which is correlated to at least some externally measurable parameter, which in some examples, includes at least one of: paradoxical respiratory effort belt signals; increased inspiratory effort; and absence of an inspiratory effort. In some examples, in a manner consistent with the previously described examples regarding a data model, a data model (e.g. a trainable machine learning model) may be used to implement method 2300.

FIG. 36 is diagram schematically representing an example method 2310, which may form part of, or be associated with, method 2300 in FIG. 35 in some examples or a more general example method of identifying sleep disordered breathing (e.g. FIGS. 1A-2B). As shown in FIG. 35, method 2300 comprises differentiating obstructive sleep apnea (OSA) from central sleep apnea (CSA) by identifying, via the sensed physiologic information (e.g. via at least an implanted accelerometer), at least two orthogonal axes in which each respective axis exhibits a first type of acceleration waveform during OSA based on a first torso motion and exhibits a second type of acceleration waveform during CSA based on a second torso motion. In some such examples, the fiducial may comprise at least one of an (relative) amplitude, a signal-to-noise (SNR) ratio, and a deviation.

In some examples, the two orthogonal axes may comprise an axis “A” and a second orthogonal axis “B”, which capture different axes of torso movement. For instance, during obstructive sleep apnea (OSA) the A and B axes exhibit a relative signal-to-noise ratio, signal amplitude, deviation, distinct signature in signal morphology, etc. that differs from the relative signal in axes A and B during central sleep apnea (CSA). The relative metric may be tuned to highlight phenomena used to distinguish OSA from CSA, particularly paradoxical breathing, such as when the chest and abdomen are moving opposite of each other, such as one contracting while the other expands (or vice versa). With this in mind, OSA and CSA exhibit different movement of the abdomen and chest (reflecting the underlying disease mechanism) and one or more axes of an accelerometer (e.g. 285 in FIG. 2B, 304A/322A in FIGS. 3A-3B) oriented relative to the orthogonal coronal, transverse, and sagittal planes will vary depending on the way the chest and abdomen move (and how they move relative to one another).

By differentiating OSA from CSA, therapy may be adapted by a clinician or by a device via auto-titration. Some combination of one or more of any of the above measures may be used to distinguish CSA from OSA to ensure that upper airway stimulation can be applied when the OSA is present, but not necessarily applied when CSA is present, in some examples.

FIG. 37 is a block diagram of an example measure types 2320, such as for an apnea-hypopnea index (AHI), an oxygen desaturation index (ODI), and/or other disease burden indicators. As shown in FIG. 37, in some examples the measure types 2320 comprise performing measurements for one of the indices (e.g. AHI or ODI) on an event-to-event basis (e.g. apnea-hypopnea (AH) to apnea-hypopnea (AH) basis) 2322, on a repeating clock basis 2324 (e.g. hourly), a rolling hour basis 2326 (e.g. continuously updating on a previous hour basis), and/or an average basis 2328 (e.g. average index score for a whole night of sleep).

The repeating clock basis 2324 may be hourly or could be any other fixed or adjustable interval. The measurement may include selectable criteria such as a threshold duration (e.g. at least 10 seconds for restricted airflow) and/or a predictive model of oxygen saturation changes during an apnea-hypopnea event.

In some examples, such measure types 2320 may be employed to identify sleep disordered breathing (SDB) via determining an apnea-hypopnea index (AHI) via computing a measure of the per hour AHI on at least one of the measure types 2322, 2324, 2326, 2328.

In some examples, such measure types 2320 may be employed to identify an oxygen desaturation index (ODI) via determining an oxygen desaturation index (ODI) via computing a measure of the per hour ODI on at least one of the measure types 2322, 2324, 2326, 2328. It will be understood that the ODI may be at least partially indicative of sleep disordered breathing (SDB), and hence identifying ODI may comprise identifying sleep disordered breathing (SDB) in some examples.

In some examples, as shown at 2330 in FIG. 38, at least some more general example methods (e.g. at least FIGS. 1A-2B) may further comprise gathering, via a control portion (e.g. 3000 in FIG. 52B) of the IMD on a periodic basis, the sensed physiologic information. In some examples, the periodic basis may comprise a single treatment period (e.g. night), a single week, and/or a selectable predetermined period (e.g. 3 days, 2 weeks, etc.). In some such examples, the relevant period during which the data is gathered is to be replicated each occasion on which data is gathered. In some examples, the gathered sensed physiologic information may comprise a statistical summary or samples (e.g. snapshots) rather than continuous data. It will be understood that in some instances, the gathering may occur on a pseudo-random non-periodic basis.

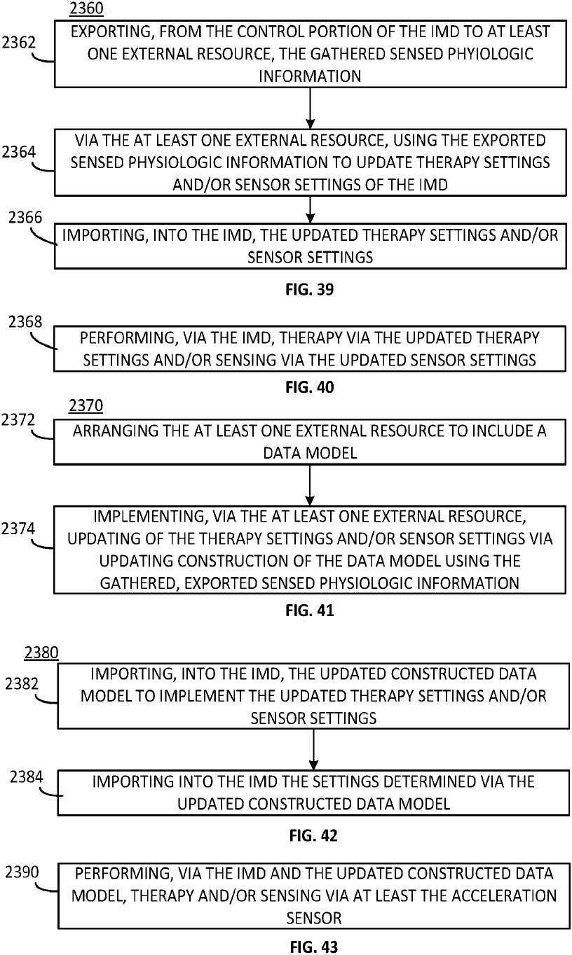

FIG. 39 is a flow diagram schematically representing an example method 2360. As shown at 2362 in FIG. 39, in some examples method 2360 comprises exporting, from the control portion (e.g. 3000 in FIG. 52B) of the IMD to at least one external resource, the gathered sensed physiologic information while at 2364, method 2360 comprises, via the at least one external resource, using the exported sensed physiologic information to update therapy settings (e.g. stimulation settings) and sensing settings of the IMD. In some such examples, the updating may comprise periodic updating or may comprise pseudo-random non-periodic updating. As shown at 2366 in FIG. 39, method 2360 comprises importing, into the IMD, the updated therapy settings and updated sensing settings.

In one aspect, this arrangement of exporting data to perform updating external of the IMD facilitates the use of larger, faster computing resources to perform the updating, which allows the IMD to use less circuitry, less logic, less power, etc. Upon the external updating, the updated settings are imported back into the IMD.

In some examples, the at least one external resource may comprise a patient remote control, a computer (e.g. laptop, desktop, etc.), a mobile computing device, and/or a clinician portal (e.g. cloud computing resource), such as but not limited the corresponding examples (e.g. 3074, 3076, 3070, 3080, 3082, etc.) as shown in FIG. 52E. The mobile computing device may comprise a tablet, phablet, personal digital assistant (PDA), phone, and the like. In some examples, the external resource also may comprise and/or be in communication with a sensor(s), which may sense any of the physiologic parameters disclosed throughout the various examples of the present disclosure. The sensors may be external, removably insertable internally within the body. In some examples, the external resource does not comprise a sensor but may receive sensed physiologic information.

In some examples, the particular types and/or locations of the at least one external resource may be chosen to balance various factors for division of processing signal information, therapy settings, sensor settings, etc. Accordingly, in some examples, the processing may be divided between inside the implant and external to the implant (e.g. on the remote, or on a PC, or in the cloud). In some examples, one possible division may comprise a portion of the implantable medical device capturing snapshots or statistical summaries of sensed information, which is then communicated to at least one external resource to be processed external to the implantable medical device (such as external to the patient’s body). The sensed information may comprise respiratory information, including but not limited to: (A) a number, type, rate of sleep disordered breathing events; (B) response to stimulation therapy; (C) sleep quality; and (D) and other information.

It will be understood that for any disease burden indicator in general or with respect to sleep disordered breathing, the sensed information communicated between the external resource and the IMD may comprise a wide variety of physiologic information extending far beyond the above-noted respiratory information.

The processed results may be communicated to the implantable medical device (IMD) to implement the therapy titration and/or sensing adjustments. In some such examples, this external updating process may be performed on a night-by-night basis (or other selectable interval, time frame) instead of the infrequent manual updates. The division of processing between the implantable medical device (IMD) and any external resource would allow the use of techniques or processing engines that might otherwise be infeasible to implement solely via the implantable medical device (IMD) due to battery power limitations and/or processing capacity/speed within the implantable medical device (IMD). The division of processing (internal vs. external) also may be chosen given communication speed constraints. Accordingly, in at least some examples, the processing location is to be selected to optimize latency, processing capacity/speed, battery longevity (e.g., IMD and remote control), communication speed, system complexity, and availability of data aggregation across multiple patients.

In some such examples regarding division of processing (e.g. between internal and external), the use of data models (e.g. machine learning techniques) may improve accuracy but may involve much high processing power demands. For example, the use of training of inputs to construct the data model may reduce battery life and increase the complexity of processing. In some examples, the reduction in battery life and/or complexity of processing may flow from a data model embodiment facilitating implementation of automatic per-patient fitting of clinically relevant detection thresholds/settings (e.g. AHI, ODI, such as compared to merely using concurrent external measurement techniques (e.g. polysomnography). Accordingly, in such situations, more processing may be performed external to the IMD.

In some examples method 2360 may further comprise and/or provide a foundation for a method 2368 (as shown in FIG. 40) of performing, via the updated settings in the IMD, therapy and/or sensing physiologic information via at least an acceleration sensor. At least some examples of such therapy (e.g. stimulation) are described in association with at least FIGS. 50-51. In some examples, the therapy may comprise applying stimulation to upper airway patency-related tissue (e.g. nerves, muscles) to treat sleep disordered breathing.

FIG. 41 is a flow diagram schematically representing an example method 2370 which comprises part of, and/or associated with, method 2360 (FIG. 39). As shown at 2372 in FIG. 41, method 2370 comprises arranging the least one external resource to include a data model while at 2374, method 2370 comprises implementing, via the at least one external resource, updating of the therapy settings (e.g. stimulation, other) and/or sensor settings by updating construction of the data model (e.g. training of a data model) using the exported gathered, sensed physiologic information.

With regard to the updating in FIGS. 39 or 41, the updating may performed on a periodic basis or other time-based basis.

FIG. 42 is a flow diagram schematically representing an example method 2380 which further comprises a part of, and/or is associated with, method 2370 (FIG. 41). As shown at 2382 in FIG. 42, method 2380 comprises importing, into the implantable medical device (IMD), the updated data model to implement the updated therapy (e.g. stimulation, other) settings and/or sensor settings. At 2384, method 2380 also may comprise importing, into the IMD, the settings determined via the updated constructed data model.

As further shown in FIG. 43, in some examples a method 2390 may further comprise a part of, and/or is associated with, at least method 2380 (FIG. 42) with method 2390 comprising performing, via the IMD and the updated constructed data model, therapy and/or sensing (e.g. via the acceleration sensor). At least some examples of such therapy is described in association with at least FIGS. 50-51, with stimulation of upper airway patency-related tissue (e.g. nerves, muscles) providing just one example of applying therapy for a disease burden.

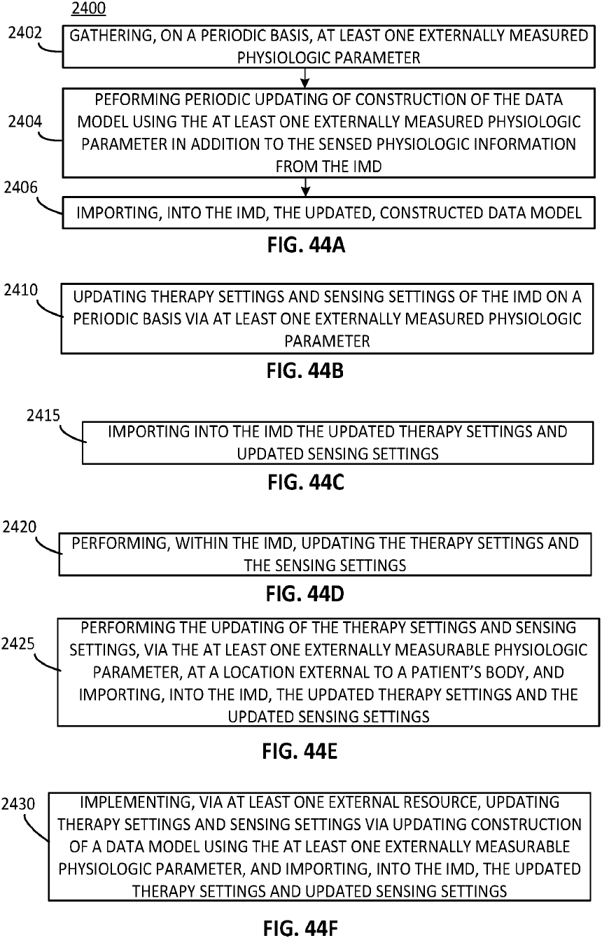

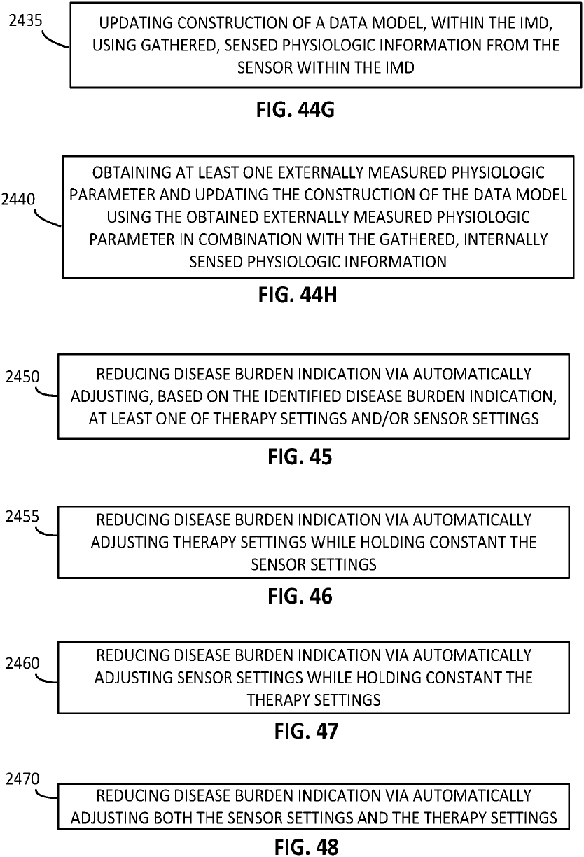

FIG. 44A is a flow diagram schematically representing an example method 2400. In some examples, method 2400 may further comprise a part of, and/or is associated with, at least the general example methods (FIGS. 1A-2B), method 2360 (FIG. 39), and the like. As shown at 2402, method 2400 comprises gathering, on periodic basis, at least one externally measured physiologic parameter, while at 2404, method 2400 comprises performing periodic updating of construction of a data model (e.g. updating training of the data model) using both the at least one externally measured physiologic parameter and internally measured data, such as physiologic information sensed by the sensor of IMD. In some examples, the sensor of the IMD comprises an implantable sensor, which in some examples comprises an implantable acceleration sensor.

In some such examples, the at least one externally measured physiologic parameter (i.e. externally measured data) matches a time period (e.g. minute-by-minute, hour-by-hour, day by day, other periods) at which the internally measured data (e.g. the gathered, sensed physiologic information in 2330 in FIG. 38) was obtained. In some examples, the time period during which both of the internally measured data and externally measured data is gathered may comprise a predetermined time window (e.g. 30 minutes each night) within a treatment period. In some examples, the externally measured data may be correlated with the internally measured data and used to refine the accuracy and effectiveness of the internally measurable data (per the IMD) when identifying disease burden indicators and/or applying therapy to such diseases. Moreover, this correlation and alignment of the externally measured data and the internally measured data may enhance the performance of the implantable medical device.

At 2406, method 2400 comprises importing, into the IMD, the updated, constructed data model. In some examples, the internally measured data may comprise at least the types, modes, etc. of physiologic information sensed via acceleration motion 1550 in FIG. 15.

With further reference to at least FIG. 44A, in some examples, at least some of the externally measurable data (e.g. at least one externally measurable physiologic parameter) may comprise at least some of the externally measurable data as previously described in association with at least FIG. 13A (1200), FIG. 14 (1530), FIG. 16A (1580), and/or FIG. 16B (1600). Accordingly, among other possible externally measurable physiologic parameters, at least some these parameters may comprise at least one: a mattress sleep sensor parameter; an RF respiration sensor parameter; a nasal airflow sensor cannula parameter; an acoustic sensor parameter; a computer vision system parameter; a respiration effort belt parameter; a blood oxygen desaturation parameter; an EEG parameter; a respiratory waveform parameter; a body position parameter; a body motion parameter; an EOG parameter; a cardiac waveform parameter; a limb movement parameter; a sleep stage parameter; an acoustic parameter; a pressure airflow sensor parameter; a thermal airflow sensor parameter; and an EMG parameter.

In some examples, the externally measurable data may be obtained via sensors in contact with the patients’ body and/or via contact-less sensors which are not in contact with the patient’s body. For example, at least one external sensor may comprise an accelerometer, piezoelectric device, and/or pressure sensor which may be worn on the body, incorporated into a mattress or other body support, etc. with such externally sensed motion/activity data being correlated with motion, activity, respiration, etc. sensed via implantable sensors, including but not limited to, an implantable accelerometer(s) within the patient’s body. In some such examples, via such example correlation arrangements, machine learning (e.g. constructing/updating a data model) can be employed and enhanced by leveraging large patient data sets regarding such externally measurable physiologic information to supplement and/or shape the scope, accuracy, and/or effectiveness of the implantably sensed physiologic information in diagnosing, monitoring, and/or treating diseases. Moreover, this correlation and alignment of the externally measured data and the internally measured data may enhance the performance of the implantable medical device.

Accordingly, via such example arrangements, from an initial situation where an implantable workflow (i.e. for using internally sensed data to identify a disease burden indicator) does not yet exist, aligning the implanted sensor data (e.g. internally sensed data) with the labeled external data (e.g. externally sensed data) may allow for the development of an implantable workflow, such as by using machine learning techniques to train a workflow (e.g. machine learning model) based on the labeled external sensor data. Once an implantable workflow has been constructed, further alignment and correlation of internally sensed data with externally sensed data may be used to enhance the effectiveness and accuracy of the implantable workflow in identifying disease burden indicator(s), application of related therapies, and/or performance of the implantable medical device.

In some examples, via at least these example arrangements in association with FIGS. 39-49 and the related examples in association with FIG. 1A-102, at least some example methods (and/or devices) provide an arrangement by which implantable sensing (which may be augmented by external sensing) acts as at least a diagnostic and/or monitoring tool to identify diseases, disease burden, etc. during sleep periods of a patient. In some such examples, this implantable sensing may comprise implantable acceleration sensing, and in some of these examples, the implantable acceleration sensing may comprise sensing rotational movement of a chest and/or abdomen to obtain respiration information among other physiologic information. While such recognition and/or therapy of disease burden is applicable to at least sleep disordered breathing and/or the specifically identify diseases in FIGS. 53A-55C, it will be understood that such example arrangements in association with at least FIGS. 39-49 may be applicable to a wide variety of diseases. For instance, one non-limiting example of identifying disease from the reference point of sleep may include identifying diabetes as a possible disease for a patient in which the presenting symptom or behavior during sleep may comprise lack of sleep quality (e.g. sleep disruption) due to restless leg syndrome, which is detectable at least via motion, activity sensed via an accelerometer (implantable, external, both). The restless leg syndrome may result from neuropathic pain, which in turn may traces its roots to diabetes. Accordingly, these example arrangements which track both implantable sensed physiologic information and externally sensed physiologic information, in relation to sleep periods (as one example), may enable example methods of identifying disease, disease burden, relationships between symptoms and disease, etc.

FIG. 44B is a diagram schematically representing an example method 2410 which may form an additional aspect of at least example method 2400 in FIG. 44A. As shown in FIG. 44B, In some examples, method 2410 comprises updating therapy settings and sensor settings of the IMD on a periodic basis (or a non-periodic basis) via at least one externally measurable physiologic parameter, such as described above in relation to at least FIG. 44A.

As shown at 2415 in FIG. 44C, in some examples, method 2400 (FIGS. 44A, 44B) may further comprise importing, into the IMD, the updated therapy settings and updated sensing settings. In some examples, the importing may be performed via a patient mobile device (e.g. mobile phone app, tablet, phablet, etc.), a patient remote, etc.

As shown at 2420 in FIG. 44D, in some examples, method 2400 (FIGS. 44A-44C) may further comprise performing, within the IMD, updating the therapy settings and sensing settings.

As shown at 2425 in FIG. 44E, in some examples, the example method(s) comprise performing the updating of the therapy setting and sensor settings (via the at least one externally measurable physiologic parameter) at a location external to a patient’s body and importing, into the IMD, the updated therapy settings and updated sensing settings.

As shown at 2430 in FIG. 44F, in some examples associated with at least FIGS. 44A-44E, a method comprises implementing, via at least one external resource, updating the therapy settings and sensor settings via updating construction of a data model using the at least one externally measurable physiologic parameter, and importing into the IMD the updated therapy settings and updated sensing settings.

As shown at 2435 in FIG. 44G, in some examples a method may comprise updating constructing a data model, within the IMD, using the gathered, sensed physiologic information. In some examples, the sensor by which the sensed physiologic information is gathered comprises an implantable sensor. In some such examples, the implantable sensor comprises at least an acceleration sensor.

As shown at 2440 in FIG. 44H, in some examples the method at 2435 (FIG. 44G) may further comprise obtaining externally sensed physiologic data (e.g. at least one externally measurable physiologic parameter) for use in updating the construction of the data model in combination with gathered, internally sensed physiologic information (e.g. sensed via/within the IMD).

FIG. 45 is a diagram schematically representing an example method 2450. In some examples, method 2450 may further comprise a part of, and/or is associated with, at least the general example methods in FIGS. 1A-2B, example methods in FIGS. 39-44, and/or other examples throughout the present disclosure. As shown in FIG. 45, method 2450 comprises reducing disease burden indication (such as sleep disordered breathing (SDB) in some examples) via automatically adjusting at least one of the therapy (e.g. stimulation, other) settings and the sensor settings of the IMD. In some such examples, the reducing may sometimes be referred to as minimizing and the increasing may sometimes be referred to as maximizing.

In some examples, the method 2450 may further comprise increasing correlation of an internally measured sensor signal (e.g. implanted accelerometer) with an externally measurable reference/parameter.

In some examples, reducing the disease burden comprises reducing sleep disordered breathing. In some examples, reducing the sleep disordered breathing (SDB) comprises reducing an apnea-hypopnea index (AHI) and/or reducing an oxygen desaturation index (ODI). In some examples, reducing a disease burden (e.g. disease burden indicator), such as but not limited to the sleep disordered breathing (SDB), comprises reducing arousals and/or increasing sleep quality, which are often highly related. In some examples, the sleep quality is at least partially determined via user feedback from a patient-reported per-night sleep quality score.

FIG. 46 is a flow diagram schematically representing an example method 2455. In some examples, method 2455 may further comprise a part of, and/or is associated with, at least the general example methods (FIGS. 1A-2B). As shown in FIG. 46, method 2455 comprises reducing disease burden indication (e.g. reducing sleep disordered breathing (SDB) indications) via automatically adjusting therapy settings while holding constant the sensor settings.

FIG. 47 is a flow diagram schematically representing an example method 2460 like method 2455, except for reducing disease burden indication (e.g. reducing sleep disordered breathing (SDB) indications) via automatically adjusting the sensor settings while holding constant the therapy settings.

FIG. 48 is a flow diagram schematically representing an example method 2470. In some examples, method 2470 may further comprise a part of, and/or is associated with, at least the general example methods (FIGS. 1A-2B) and may comprise aspects of methods described in association with at least FIGS. 45-47. As shown in FIG. 48, method 2470 comprises reducing a disease burden indicator via automatically adjusting both the therapy settings and the sensor settings. In some such examples, the automatic adjustment may be simultaneously performed for both therapy application and sensing. In some examples, the disease burden indicator may comprise sleep disordered breathing (SDB) and/or another disease burden indicator (such as but not limited to FIGS. 53A-55C).

In some such examples, reducing the disease burden indication (e.g. SDB behavior, other) via automatically adjusting both the therapy settings and the sensing settings may be performed to optimize total therapy duty cycle, and wherein the automatically adjusting further comprises, in the absence of detecting disease burden (e.g. SDB events), reducing the therapy (e.g. stimulation) duty cycle. In some such examples, reducing therapy (e.g. stimulation) duty cycle in at least this context may reduce power consumption, reduce tissue (e.g. nerve, muscle) fatigue, and/or enhance future therapy applications (e.g. for sleep disordered breathing, next-breath prediction to correctly stimulate before inspiration begins).

Therapy settings and/or sensing settings may be selected by the device from a list of sets (each set containing one or more settings and/or ranges of settings) previously selected by the clinician.

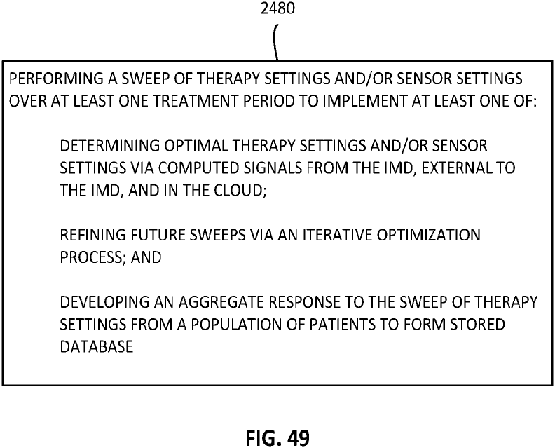

FIG. 49 schematically represents an example method 2480. In some examples, method 2480 may further comprise a part of, and/or is associated with, at least the general example methods (FIGS. 1A-2B) as well as at least some of the methods described in association with at least FIGS. 38-48. As shown in FIG. 49, method 2480 comprises performing a sweep of therapy settings and/or sensor settings over at least one treatment period to implement at least one of: (A) determining optimal therapy settings and/or sensor settings via computed signals from the IMD, external to the IMD, and the cloud; (B) refining future sweeps via an iterative optimization process; and (C) developing an aggregate response to the sweep of therapy settings from a population of patients to form a stored database.

In some examples, the optimal therapy settings and/or sensor settings may be limited within a range set by a clinician to ensure appropriate therapy and/or sensing.

In some examples, pursuant to method 2480 (FIG. 49) a database may be used for retroactive data analysis to determine optimal parameters for therapy, such as upper airway patency-related tissue stimulation in the case of sleep disordered breathing. In some examples, parametric sweeps may alternatively be performed on subgroups of consented patients to explore clinical or research questions regarding therapy application (e.g. stimulation of the upper airway patency-related tissue in some examples). In some examples, further measurements may be made, such as therapy threshold, therapy effectiveness, or impedance. In some examples, these further measurements may be made in a range of electrode configurations and across multiple patients to develop and refine an anatomical model of the tissue to which therapy is being applied (e.g. a hypoglossal nerve or other upper airway patency-related tissue, in some examples) and enhance surgical implant practice.

In some examples, the optimal therapy settings and/or sensing settings (from a full population of patients) may be sent back to the IPG to improve patient therapy. In some examples, an iterative process may be used to refine the settings over time.

In some examples, one of the previously described example methods of identifying disease burden indication (e.g. sleep disordered breathing (SDB)) may be used to measure a baseline rate of disease burden indication (e.g. sleep disordered breathing (SDB) per AHI, ODI) when a patient is not using therapy. In some such examples, this measurement may take place during sleep periods or other periods occurring during a patient’s post-implant recovery portion or when a patient is sleeping (or during other periods) but does not enable therapy. Data relating to the measured baseline rate of disease burden indication (e.g. sleep disordered breathing (SDB)) may be displayed to the patient and/or a clinician, with such data demonstrating the difference between using therapy and not using therapy. Moreover, after collecting this baseline data, in some examples a predictive responder score may be computed that predicts the reduction in behaviors characteristic of disease burden after they begin using therapy. In the example of sleep disordered breathing, there may be a predicted reduction in implant-measured AHI/ODI/arousal rates after they begin using therapy. In some examples, this responder score may allow earlier and/or more frequent clinical intervention if the patient is predicted to not respond or respond poorly to therapy based on particular sensor settings and/or therapy settings. In some examples, the predictive model may be trained on a full patient population or on a subset of the full patient population.

FIG. 50 is a diagram including a front view of an example device 2811 (and/or example method) implanted within a patient’s body 2800. In some examples, the device 2811 may comprise an implantable medical device (IMD) 2833 such as (but not limited to) an implantable pulse generator (IPG) with device 2833 including a sensor 2835. In some examples, device 2833 comprises at least some of substantially the same features and attributes as IMD 283 (including acceleration sensor 285), as previously described in association with at least FIG. 2B). Accordingly, in some examples, sensor 2835 may comprise a sensor (e.g. 285 in FIG. 2B, 304A/322A in FIGS. 3A-3B,, etc.) having at least some of substantially the same features and attributes as previously described in association with at least FIGS. 1-49 and/or FIG. 56A-102. Via such example sensing arrangements, the device 2833 may determine different types of physiologic information, which includes but is not limited to respiration information via sensing rotational movement of the patient’s chest wall during breathing, such as but not limited to when in a sleeping body position during a treatment period.

As further shown in FIG. 50, device 2811 comprises a lead 2817 including a lead body 2818 for chronic implantation (e.g. subcutaneously via tunneling or other techniques) and to extend to a position adjacent a nerve (e.g. hypoglossal nerve 2805 (or other upper airway patency-related tissue) and/or phrenic nerve 2806). The lead 2817 may comprise a stimulation electrode to engage the nerve (e.g. 2805, 2806) for stimulating the nerve to treat a physiologic condition, such as sleep disordered breathing like obstructive sleep apnea, central sleep apnea, multiple-type sleep apneas, etc. The IMD 2833 may comprise circuitry, power element, etc. to support control and operation of both the sensor 2835 and the stimulation electrode 2812 (via lead 2117). In some examples, such control, operation, etc. may be implemented, at least in part, via a control portion (and related functions, portions, elements, engines, parameters, etc.) such as described later in association with at least FIGS. 52A-52E.

It will be understood that the lead 2817 may be implanted with regard to other tissues (e.g. FIG. 1B) to apply therapy to treat at least some other diseases, such as at least some of the diseases described in association with at least FIGS. 53A-55C.

With regard to the various examples of the present disclosure, in some examples, delivering stimulation to an upper airway patency nerve 2805 (e.g. a hypoglossal nerve, other nerves) via the stimulation electrode 2812 is to cause contraction of upper airway patency-related muscles, which may cause or maintain opening of the upper airway (2808) to prevent and/or treat obstructive sleep apnea. Similarly, in some examples such electrical stimulation may be applied to a phrenic nerve 2806 via the stimulation electrode 2812 to cause contraction of the diaphragm as part of preventing or treating at least central sleep apnea. It will be further understood that some example methods may comprise treating both obstructive sleep apnea and central sleep apnea, such as but not limited to, instances of multiple-type sleep apnea in which both types of sleep apnea may be present at least some of the time. In some such instances, separate stimulation leads 2817 may be provided or a single stimulation lead 2817 may be provided but with a bifurcated distal portion with each separate distal portion extending to a respective one of the hypoglossal nerve 2805 (or other nerve) and the phrenic nerve 2806.

In some such examples, the contraction of the hypoglossal nerve and/or contraction of the phrenic nerve caused by electrical stimulation comprises a suprathreshold stimulation, which is in contrast to a subthreshold stimulation (e.g. mere tone) of such muscles. In one aspect, a suprathreshold intensity level corresponds to a stimulation signal amplitude greater than the nerve excitation threshold, such that the suprathreshold stimulation may provide for higher degrees (e.g. maximum, other) of upper-airway clearance (i.e. patency) and sleep apnea therapy efficacy.

In some examples, a target intensity level of stimulation signal amplitude is selected, determined, implemented, etc. without regard to intentionally establishing a discomfort threshold of the patient (such as in response to such stimulation). Stated differently, in at least some examples, a target intensity level of stimulation may be implemented to provide the desired efficacious therapeutic effect in reducing sleep disordered breathing (SDB) without attempting to adjust or increase the target intensity level according to (or relative to) a discomfort threshold.

In some examples, the treatment period (during which stimulation may be applied at least part of the time) may comprise a period of time beginning with the patient turning on the therapy device and ending with the patient turning off the device. In some examples, the treatment period may comprise a selectable, predetermined start time (e.g. 10 p.m.) and selectable, predetermined stop time (e.g. 6 a.m.). In some examples, the treatment period may comprise a period of time between an auto-detected initiation of sleep and auto-detected awake-from-sleep time. With this in mind, the treatment period corresponds to a period during which a patient is sleeping such that the stimulation of the upper airway patency-related nerve and/or central sleep apnea-related nerve is generally not perceived by the patient and so that the stimulation coincides with the patient behavior (e.g. sleeping) during which the sleep disordered breathing behavior (e.g. central or obstructive sleep apnea) would be expected to occur.

In some examples the initiation or termination of the treatment period may be implemented automatically based on sensed sleep state information, which in turn may comprise sleep stage information.

To avoid enabling stimulation prior to the patient falling asleep, in some examples stimulation can be enabled after expiration of a timer started by the patient (to enable therapy with a remote control), or enabled automatically via sleep stage detection. To avoid continuing stimulation after the patient wakes, stimulation can be disabled by the patient using a remote control, or automatically via sleep stage detection. Accordingly, in at least some examples, these periods may be considered to be outside of the treatment period or may be considered as a startup portion and wind down portion, respectively, of a treatment period.

In some examples, stimulation of an upper airway patency-related nerve may be performed via open loop stimulation. In some examples, the open loop stimulation may refer to performing stimulation without use of any sensory feedback of any kind relative to the stimulation.

In some examples, the open loop stimulation may refer to stimulation performed without use of sensory feedback by which timing of the stimulation (e.g. synchronization) would otherwise be determined relative to respiratory information (e.g. respiratory cycles). However, in some such examples, some sensory feedback may be utilized to determine, in general, whether the patient should receive stimulation based on a severity of sleep apnea behavior.

Conversely, in some examples and as previously described in relation to at least several examples, stimulation of an upper airway patency-related nerve may be performed via closed loop stimulation. In some examples, the closed loop stimulation may refer to performing stimulation at least partially based on sensory feedback regarding parameters of the stimulation and/or effects of the stimulation.

In some examples, the closed loop stimulation may refer to stimulation performed via use of sensory feedback by which timing of the stimulation (e.g. synchronization) is determined relative to respiratory information, such as but not limited to respiratory cycle information, which may comprise onset, offset, duration, magnitude, morphology, etc. of various features of the respiratory cycles, including but not limited to the inspiratory phase, expiratory active phase, etc. In some examples, the respiration information excludes (i.e. is without) tracking a respiratory volume and/or respiratory rate. In some examples, stimulation based on such synchronization may be delivered throughout a treatment period or throughout substantially the entire treatment period. In some examples, such stimulation may be delivered just during a portion or portions of a treatment period.

In some examples of “synchronization”, synchronization of the stimulation relative to the inspiratory phase may extend to a pre-inspiratory period and/or a post-inspiratory phase. For instance, in some such examples, a beginning of the synchronization may occur at a point in each respiratory cycle which is just prior to an onset of the inspiratory phase. In some examples, this point may be about 200 milliseconds, or 300 milliseconds prior to an onset of the inspiratory phase.

In some examples in which the stimulation is synchronous with at least a portion of the inspiratory phase, the upper airway muscles are contracted via the stimulation to ensure they are open at the time the respiratory drive controlled by the central nervous system initiates an inspiration (inhalation). In some such examples, in combination with the stimulation occurring during the inspiratory phase, example implementation of the above-noted pre-inspiratory stimulation helps to ensure that the upper airway is open before the negative pressure of inspiration within the respiratory system is applied via the diaphragm of the patient’s body. In one aspect, this example arrangement may minimize the chance of constriction or collapse of the upper airway, which might otherwise occur if flow of the upper airway flow were too limited prior to the full force of inspiration occurring.

In some such examples, the stimulation of the upper airway patency-related nerve may be synchronized to occur with at least a portion of the expiratory period.

With regard to at least the methods of treating sleep apnea as previously described in association with at least FIGS. 1-51, at least some such methods may comprise performing the delivery of stimulation to the upper airway patency-related first nerve without synchronizing such stimulation relative to a portion of a respiratory cycle. In some instances, such methods may sometimes be referred to as the previously described open loop stimulation.

In some examples, the term “without synchronizing” may refer to performing the stimulation independently of timing of a respiratory cycle. In some examples, the term “without synchronizing” may refer to performing the stimulation while being aware of respiratory information but without necessarily triggering the initiation of stimulation relative to a specific portion of a respiratory cycle or without causing the stimulation to coincide with a specific portion (e.g. inspiratory phase) of respiratory cycle.

In some examples, in this context the term “without synchronizing” may refer to performing stimulation upon the detection of sleep disordered breathing behavior (e.g. obstructive sleep apnea events) but without necessarily triggering the initiation of stimulation relative to a specific portion of a respiratory cycle or without causing the stimulation to coincide with the inspiratory phase. At least some such examples may be described in Wagner et al., STIMULATION FOR TREATING SLEEP DISORDERED BREATHING, published as US 2018/0117316 on May 3, 2018, and which is incorporated by reference herein in its entirety.

In some examples, while open loop stimulation may be performed continuously without regard to timing of respiratory information (e.g. inspiratory phase, expiratory phase, etc.) such an example method and/or system may still comprise sensing respiration information for diagnostic data and/or to determine whether (and by how much) the continuous stimulation should be adjusted. For instance, via such respiratory sensing, it may be determined that the number of sleep disordered breathing (SDB) events are too numerous (e.g. an elevated AHI) and therefore the intensity (e.g. amplitude, frequency, pulse width, etc.) of the continuous stimulation should be increased or that the SDB events are relative low such that the intensity of the continuous stimulation can be decreased while still providing therapeutic stimulation. It will be understood that via such respiratory sensing, other SDB-related information may be determined which may be used for diagnostic purposes and/or used to determine adjustments to an intensity of stimulation, initiating stimulation, and/or terminating stimulation to treat sleep disordered breathing. It will be further understood that such “continuous” stimulation may be implemented via selectable duty cycles, train of stimulation pulses, selective activation of different combinations of electrodes, etc.

In some examples of open loop stimulation or closed loop stimulation, some sensory feedback may be utilized to determine, in general, whether the patient should receive stimulation based on a severity of sleep apnea behavior. In other words, upon sensing that a certain number of sleep apnea events are occurring, the device may implement stimulation.

Some non-limiting examples of such devices and methods to recognize and detect the various features and patterns associated with respiratory effort and flow limitations include, but are not limited to: Christopherson et al. US 8,938,299, SYSTEM FOR TREATING SLEEP DISORDERED BREATHING, issued Jan. 20, 2015; Christopherson U.S. Pat. 5,944,680, titled RESPIRATORY EFFORT DETECTION METHOD AND APPARATUS; and Testerman U.S. Pat. 5,522,862, titled METHOD AND APPARATUS FOR TREATING OBSTRUCTIVE SLEEP APNEA, each of which is hereby incorporated by reference herein in their entirety. Moreover, in some examples various stimulation methods may be applied to treat obstructive sleep apnea, which include but are not limited to: Ni et al., SYSTEM FOR SELECTING A STIMULATION PROTOCOL BASED ON SENSED RESPIRATORY EFFORT, which issued as US 10,583,297 on Mar. 10, 2020; Christopherson et al. US 8,938,299, SYSTEM FOR TREATING SLEEP DISORDERED BREATHING, issued Jan. 20, 2015; Christopherson U.S. Pat. 5,944,680, titled RESPIRATORY EFFORT DETECTION METHOD AND APPARATUS; and Wagner et al. STIMULATION FOR TREATING SLEEP DISORDERED BREATHING, published as US 2018/0117316 on May 3, 2018, each of which is hereby incorporated by reference herein in their entirety.

In some examples, the example stimulation element(s) 2812 shown in FIG. 50 may comprise at least some of substantially the same features and attributes as described in Bonde et al. U.S. 8,340,785, SELF EXPANDING ELECTRODE CUFF, issued on December 25, 2102 and Bonde et al. U.S. 9,227,053, SELF EXPANDING ELECTRODE CUFF, issued on Jan. 5, 2016, Johnson et al. U.S. 8,934,992, NERVE CUFF issued on Jan. 13, 2015, and Rondoni et al. CUFF ELECTRODE, WO 2019/032890 published on Feb. 14, 2019, and filed as U.S. application Serial No. 16/485,954 on Aug. 14, 2019 which published as U.S. 2020-0230412 on Jul. 23, 2020, each of which are incorporated by reference herein in their entirety. Moreover, in some examples a stimulation lead 2817, which may comprise one example implementation of a stimulation element, may comprise at least some of substantially the same features and attributes as the stimulation lead described in U.S. Pat. No. 6,572,543 to Christopherson et al., and which is incorporated by reference herein in its entirety.

In some examples, the stimulation electrode 2812 may be delivered transvenously, percutaneously, etc. In some such examples, a transvenous approach may comprise at least some of substantially the same features and attributes as described in Ni et al., TRANSVENOUS METHOD OF TREATING SLEEP APNEA, issued as U.S. 9,889,299 on Feb. 13, 2018, and which is hereby incorporated by reference. In some such examples, a percutaneous approach may comprise at least some of substantially the same features and attributes as described in Christopherson et al., PERCUTANEOUS ACCESS FOR SYSTEMS AND METHODS OF TREATING SLEEP APNEA, issued as U.S. 9,486,628 on Nov. 8, 2016, and which is hereby incorporated by reference.

As further shown in the diagram of FIG. 50, in some examples device 2811 (FIG. 50) may be implemented with additional sensors 2820, 2830, etc. to sense additional physiologic information, such as but not limited to, further respiratory information via sensing transthoracic bio-impedance, pressure sensing, etc. in order to complement the respiration information sensed via acceleration sensor 2835 (or other sensor). In some examples, one or both of the sensors 2820, 2830 may comprise sensor electrodes. In some examples, stimulation electrode 2812 also may act, in some examples, as a sensing electrode. In some examples, at least a portion of housing of the device 2833 also may comprise a sensor or at least an electrically conductive portion (e.g. electrode) to work in cooperation with sensing electrodes (e.g. 2820, 2830, and/or 2812) to implement at least some sensing arrangements to sense bioimpedance, ECG, etc.

FIG. 51 is a diagram schematically representing an example treatment device 2819A comprising at least some of substantially the same features and attributes as the treatment device 2811 in FIG. 50, except with the IMD 2833 implemented as a microstimulator 2819B. In some examples, the microstimulator 2819B may be chronically implanted (e.g. percutaneously, subcutaneously, transvenously, etc.) in a head-and-neck region 2803 as shown in FIG. 51, or in a pectoral region 2801. In some examples, as part of the treatment device 2819A, the microstimulator 2819B may be in wired or wireless communication with stimulation electrode 2812. In some examples, as part of the treatment device 2819A, the microstimulator 2819B also may incorporate sensor 2835 or be in wireless or wired communication with a sensor 2835 located separately from a body of the microstimulator 2819B. When wireless communication is employed for sensing and/or stimulation, the microstimulator 2819B may be referred to as leadless implantable medical device for purposes of sensing and/or stimulation. In some examples, the microstimulator 2819B may be in close proximity to a target nerve 2805.

In some examples, the microstimulator 2819B (and associated elements) and/or treatment device 2819A may comprise at least some of substantially the same features and attributes as described and illustrated in Rondoni et al, MICROSTIMULATION SLEEP DISORDERED BREATHING (SDB) THERAPY DEVICE, published May 26, 2017 as WO 2017/087681, and published as U.S. 2020-0254249 on Aug. 13, 2020 from U.S. Application Serial No. 15/774,471 filed on May 8, 2018, both of which are incorporated by reference herein.

In a manner similar to FIG. 50, it will be understood that the microstimulator 2819B may be implanted with regard to other tissues (e.g. FIG. 1B) to apply therapy to treat at least some other diseases, such as at least some of the diseases described in association with at least FIGS. 53A-55C.

FIG. 52A is a block diagram schematically representing an example care engine 2900. In some examples, the care engine 2900 may form part of a control portion 3000, as later described in association with at least FIG. 52B, such as but not limited to comprising at least part of the instructions 3011 and/or information 3012. In some examples, the care engine 2900 may be used to implement at least some of the various example devices and/or example methods of the present disclosure as previously described in association with FIGS. 1-51 and/or as later described in association with FIG. 52B-102. In some examples, the care engine 2900 (FIG. 52A) and/or control portion 3000 (FIG. 52B) may form part of, and/or be in communication with, a pulse generator (e.g. 283 in FIG. 2A, 2833 in FIG. 50) or microstimulator (e.g. 2819B in FIG. 51). In some examples, the care engine 2900 (FIG. 52A) may be form part of, or be in communication with, one of the devices (e.g. 3060, 3070, 3074, 3076, 3080) in the arrangement of FIG. 52E.

In some examples, the care engine 2900 may comprise and/or be implemented via at least some of substantially the same features and attributes as the care engine 7500 later described in association with at least FIG. 75E.

As shown in FIG. 52A, in some examples the care engine 2900 comprises a sensing engine 2910, a respiration engine 2912, a data model engine 2914, a sleep disordered breathing (SDB) engine 2916, and/or a stimulation engine 2918.

In one aspect, at least the sensing engine 2910 of care engine 2900 in FIG. 52A directs the sensing of information, and/or receives, tracks, and/or evaluates sensed information obtained via one or more of the sensors (including accelerometer 285, 104A, 122A), sensing elements, sensing modalities, etc. as previously described in association with at least FIGS. 1-51, with care engine 2900 employing such information to determine respiration information, blood oxygen desaturation, sleep disordered breathing, arousals, among other actions, functions, etc. as further described below.

As shown in FIG. 52A, in some examples, care engine 2900 may comprise a respiration engine 2912. In at least some examples, in general terms respiration engine 2912 may direct determining respiration information, including sensing of, and/or receiving, tracking, and/or evaluating respiratory morphology, including phase information, general patterns and/or specific fiducials within a respiratory signal. In some examples, the respiration engine 2912 may operate in cooperation with, or as part of sensing engine 2910 in FIG. 51A, which particularly includes (among other things) obtaining or sensing acceleration signal information to sense rotational movement of a patient’s chest. Accordingly, in some examples the respiration engine 2912 comprises a feature extraction portion to determine respiratory morphology (including phase information) from the sensed acceleration signals regarding rotational movement of the chest wall. With this in mind, at least some aspects of such respiratory morphology determined, monitored, received, etc. via respiration engine 2912 may comprise inspiration phase morphology, expiration active phase morphology, and/or expiratory pause phase morphology, with at least some of these attributes being illustrated in association with at least FIG. 3C. In some examples, the respective inspiration morphology, expiratory active morphology, and/or expiratory pause morphology may comprise amplitude, duration, peak, onset, and/or offset of the respective inspiratory and/or expiratory phases of the patient’s respiratory cycle. With this in mind, in some examples determining the respiratory morphology comprises identifying within the respiratory morphology a respiratory period, which includes the inspiratory phase, the expiratory active phase, and the expiratory pause phase. Accordingly, the respiratory period corresponds to a duration of a respiratory cycle, with this duration comprising a sum of a duration of the inspiratory phase, a duration of the expiratory active phase, and a duration of the expiratory pause phase. In some examples, the detected respiration morphology may comprise transition morphology such as an inspiration-to-expiration transition and/or an expiration-to-inspiration transition.

In some examples, the detected respiration morphology comprises detection (within the respiratory waveform morphology) of a start of the inspiratory phase, i.e. onset of inspiration. In some examples, this start of the inspiratory phase also may correspond to an expiration-to-inspiration transition. In some examples, a method of detecting the start of the inspiratory phase within the detected respiratory waveform morphology further comprises performing the detection without identifying an end (e.g. offset) of the inspiratory phase, thereby improving the accuracy of identification (of the start of the inspiratory phase) in the presence of noise, in contrast to identification of more than one phase transition (e.g. inspiratory-to-expiratory or expiratory-to-inspiratory) per respiratory cycle where each transition may be subject to mis-identification due to noise. In some examples, the end (e.g. offset) of the inspiratory phase corresponds to a start (e.g. onset) of the expiratory active phase.

In some examples, the respiration engine 2912 may identify (within the respiratory waveform morphology) a respiratory peak pressure, which predictably occurs a short interval after the end of inspiration and which may be used in aspects of respiration detection and related parameters. In one aspect, this arrangement may enhance the accuracy of identification (of an inspiratory-to-expiratory transition, end of inspiration, etc.) in the presence of noise due to the ease of identification of the relatively high mathematical derivative of the pressure signal associated with the interval following the end of inspiration.

In some examples, the respiration engine 2912 may identify (within the respiratory waveform morphology) an end of expiration, which may be used in some aspects of respiration detection and related parameters.

In some examples, the respiration engine 2912 may comprise a slope inversion parameter to enhance tracking of the phases (e.g. inspiratory, etc.) of the determined respiration information regardless of whether the signal may be inverted relative to a default positive slope, as previously described in various examples of the present disclosure such that the respiration information may be reliably determined regardless of the patient’s rotation in space and/or relative to the gravity vector (in at least some examples). In this regard, it will be noted that the determination of and/or use of the respiration information does not depend on which polarity the signal exhibits, but rather depends, at least partially, on the morphology of the respective phases (e.g. inspiratory, expiratory active, expiratory pause).

As further shown in FIG. 52A, in some examples the care engine 2900 comprises a SDB parameters engine 2916 to direct sensing of, and/or receive, track, evaluate, etc. parameters particularly associated with sleep disordered breathing (SDB) care. In some examples, the SDB parameters may comprise blood oxygen desaturation. For instance, in some examples, the SDB parameters engine 2916 may comprise a sleep quality portion to sense and/or track sleep quality of the patient in particular relation to the sleep disordered breathing behavior of the patient. Accordingly, in some examples the sleep quality portion comprises an arousals parameter to sense and/or track arousals caused by sleep disordered breathing (SDB) events with the number, frequency, duration, etc. of such arousals being indicative of sleep quality (or lack thereof). In some examples, the sleep quality portion comprises a state parameter to sense and/or track the occurrence of various sleep states (including sleep stages) of a patient during a treatment period or over a longer period of time.

In some examples, the SDB parameters engine 2916 comprises an AHI parameter to sense and/or track apnea-hypopnea index (AHI) information, which may be indicative of the patient’s sleep quality. In some examples, the AHI information is obtained via a sensing element, such as one or more of the various sensing types, modalities, etc., which may be implemented as described in various examples of the present disclosure.

As further shown in FIG. 52A, in some examples care engine 2900 comprises a stimulation engine 2918 to control stimulation of target tissues, such as but not limited to an upper airway patency nerve (e.g. hypoglossal nerve) and/or a phrenic nerve, to treat sleep disordered breathing (SDB) behavior. In some examples, the stimulation engine 2918 comprises a closed loop parameter to deliver stimulation therapy in a closed loop manner such that the delivered stimulation is in response to and/or based on sensed patient physiologic information.

In some examples, the closed loop parameter may be implemented using the sensed information to control the particular timing of the stimulation according to respiratory information, in which the stimulation pulses are triggered by or synchronized with specific portions (e.g. inspiratory phase) of the patient’s respiratory cycle(s). In some such examples and as previously described, this respiratory information may be determined via the sensors, sensing elements, devices, sensing portions, as previously described in association with at least FIGS. 1-51.

In some examples in which the sensed physiologic information enables determining at least respiratory phase information, the closed loop parameter may be implemented to initiate, maintain, pause, adjust, and/or terminate stimulation therapy based on (at least) the determined respiratory phase information per respiration engine 2912 and/or sensing engine 2910.

In some examples, the stimulation is started prior to an onset of the inspiratory phase (Ti in FIG. 3C) and the stimulation is stopped exactly at the end of the inspiratory phase or stopped just after the end of the inspiratory phase.

In some examples the stimulation engine 2918 comprises an open loop parameter by which stimulation therapy is applied without a feedback loop of sensed physiologic information. In some such examples, in an open loop mode the stimulation therapy is applied during a treatment period without (e.g. independent of) information sensed regarding the patient’s sleep quality, sleep state, respiratory phase, AHI, etc. In some such examples, in an open loop mode the stimulation therapy is applied during a treatment period without (i.e. independent of) particular knowledge of the patient’s respiratory cycle information.

However, in some such examples, some sensory feedback may be utilized to determine, in general, whether the patient should receive stimulation based on a severity of sleep apnea behavior.

In some examples the stimulation engine 2918 comprises an auto-titration parameter by which an intensity of stimulation therapy can be automatically titrated (i.e. adjusted) to be more intense (e.g. higher amplitude, greater frequency, and/or greater pulse width) or to be less intense within a treatment period.

In some such examples and as previously described, such auto-titration may be implemented based on sleep quality, which may be obtained via sensed physiologic information, in some examples. It will be understood that such examples may be employed with synchronizing stimulation to sensed respiratory information (i.e. closed loop stimulation) or may be employed without synchronizing stimulation to sensed respiratory information (i.e. open loop stimulation).

In some examples, at least some aspects of the auto-titration parameter of the stimulation engine 2918 may comprise, and/or may be implemented, via at least some of substantially the same features and attributes as described in Christopherson et al. US 8,938,299, SYSTEM FOR TREATING SLEEP DISORDERED BREATHING, issued Jan. 20, 2015, and which is hereby incorporated by reference in its entirety.

Moreover, with further reference to FIG. 52A, in some examples the above-mentioned electrocardiogram (ECG), ballistocardiograph sensing (BCG), seismocardiograph sensing (SCG), and/or accelerocardiograph sensing (ACG) (as previously described in association with at least FIG. 14) may be employed in combination with the sensing of acceleration-based inclination angles (based on rotational movement of the rib cage during breathing) described throughout the various examples of the present disclosure, as noted in association with at least sensing engine 2910 of care engine 2900. In one aspect, the ECG, SCG, BCG, and/or ACG sensing may be used to perform sensing of Respiratory Sinus Arrhythmia (RSA) and by which respiration detection may be performed. In some such examples, the sensed RSA may be used to identify an inspiratory phase, expiratory active phase, and/or expiratory pause phase of a respiratory cycle (such as represented in FIG. 3C) and/or may be used to distinguish the respective phases from each other. In some such examples, such identifying and/or such distinguishing may be performed via the identifying an R—R interval to determine the sensed RSA, in which the R-R interval is shorter during inspiration and the R-R interval is faster during expiration.

It will be understood that the care engine 2900 may be implemented more generally in association with the various diseases associated with the disease burden indicators in addition to (or other than) sleep disordered breathing as described in association with at least FIGS. 1A-12B, FIG. 13A-51, and FIGS. 53A-55C. In some such examples, the stimulation engine 2918 may more generally represent a therapy application engine, while the SDB parameters engine 2916 more generally represent a disease burden indication parameters engine, and the respiration engine 2912 may more generally represent at least one physiologic parameter primarily associated with the particular disease.

FIG. 52B is a block diagram schematically representing an example control portion 3000. In some examples, control portion 3000 provides one example implementation of a control portion forming a part of, implementing, and/or generally managing sensors, sensing element, respiration determination elements, stimulation elements, power/control elements (e.g. pulse generator), data models, devices, user interfaces, instructions, information, engines, elements, functions, actions, and/or methods, as described throughout examples of the present disclosure in association with FIGS. 1-52A and 52C-101.

In some examples, control portion 3000 includes a controller 3002 and a memory 3010. In general terms, controller 3002 of control portion 3000 comprises at least one processor 3004 and associated memories. The controller 3002 is electrically couplable to, and in communication with, memory 3010 to generate control signals to direct operation of at least some of the sensors, sensing element, respiration determination elements, stimulation elements, power/control elements (e.g. pulse generators), devices, user interfaces, instructions, information, engines, elements, functions, actions, and/or methods, as described throughout examples of the present disclosure. In some examples, these generated control signals include, but are not limited to, employing instructions 3011 and/or information 3012 stored in memory 3010 to at least determining respiration information of a patient. Such determination of respiration information may comprise part of identifying sleep disordered breathing (SDB) and directing and managing treatment of sleep disordered breathing such as obstructive sleep apnea, hypopnea, and/or central sleep apnea. In some instances, the controller 3002 or control portion 3000 may sometimes be referred to as being programmed to perform the above-identified actions, functions, etc. such that the controller 3002, control portion 3000 and any associated processors may sometimes be referred to as being a special purpose computer, control portion, controller, or processor. In some examples, at least some of the stored instructions 3011 are implemented as, or may be referred to as, a care engine, a sensing engine, respiration determination engine, monitoring engine, and/or treatment engine. In some examples, at least some of the stored instructions 3011 and/or information 3012 may form at least part of, and/or, may be referred to as a care engine, sensing engine, respiration determination engine, monitoring engine, and/or treatment engine.

In response to or based upon commands received via a user interface (e.g. user interface 3040 in FIG. 52D) and/or via machine readable instructions, controller 3002 generates control signals as described above in accordance with at least some of the examples of the present disclosure. In some examples, controller 3002 is embodied in a general purpose computing device while in some examples, controller 3002 is incorporated into or associated with at least some of the sensors, sensing element, respiration determination elements, stimulation elements, power/control elements (e.g. pulse generators), devices, user interfaces, instructions, information, engines, functions, actions, and/or method, etc. as described throughout examples of the present disclosure.

For purposes of this application, in reference to the controller 9802, the term “processor” shall mean a presently developed or future developed processor (or processing resources) that executes machine readable instructions contained in a memory. In some examples, execution of the machine readable instructions, such as those provided via memory 3010 of control portion 3000 cause the processor to perform the above-identified actions, such as operating controller 3002 to implement the sensing, monitoring, determining respiration information, stimulation, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 3010. In some examples, the machine readable instructions may comprise a sequence of instructions, a processor-executable machine learning model, or the like. In some examples, memory 3010 comprises a computer readable tangible medium providing non-volatile storage of the machine readable instructions executable by a process of controller 3002. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 3002 may be embodied as part of at least one application-specific integrated circuit (ASIC), at least one field-programmable gate array (FPGA), and/or the like. In at least some examples, the controller 3002 is not limited to any specific combination of hardware circuitry and machine readable instructions, nor limited to any particular source for the machine readable instructions executed by the controller 3002.

In some examples, control portion 3000 may be entirely implemented within or by a stand-alone device.

In some examples, the control portion 3000 may be partially implemented in one of the sensors, sensing element, respiration determination elements, monitoring devices, stimulation devices, apnea treatment devices (or portions thereof), etc. and partially implemented in a computing resource (e.g. at least one external resource) separate from, and independent of, the apnea treatment devices (or portions thereof) but in communication with the apnea treatment devices (or portions thereof). For instance, in some examples control portion 3000 may be implemented via a server accessible via the cloud and/or other network pathways. In some examples, the control portion 3000 may be distributed or apportioned among multiple devices or resources such as among a server, an apnea treatment device (or portion thereof), and/or a user interface.

In some examples, control portion 3000 includes, and/or is in communication with, a user interface 3040 as shown in FIG. 52D.

FIG. 52C is a diagram schematically illustrating at least some example arrangements of a control portion 3020 by which the control portion 3000 (FIG. 52B) can be implemented, according to one example of the present disclosure. In some examples, control portion 3020 is entirely implemented within or by an implantable pulse generator (IPG) 3025, which has at least some of substantially the same features and attributes as a pulse generator (e.g. power/control element) as previously described throughout the present disclosure. In some examples, control portion 3020 is entirely implemented within or by a remote control 3030 (e.g. a programmer) external to the patient’s body, such as a patient control 3032 and/or a physician control 3034. In some examples, the control portion 3000 is partially implemented in the IPG 3025 and partially implemented in the remote control 3030 (at least one of patient control 3032 and physician control 3034). In some examples, the control portion 3000 is at least partially implemented via a clinician portal 3036, which may or may not be in complementary relation with elements 3025 and 3030.

FIG. 52D is a block diagram schematically representing user interface 3040, according to one example of the present disclosure. In some examples, user interface 3040 forms part or and/or is accessible via a device external to the patient and by which the therapy system may be at least partially controlled and/or monitored. The external device which hosts user interface 3040 may be a patient remote (e.g. 3032 in FIG. 52C), a physician remote (e.g. 3034 in FIG. 52C) and/or a clinician portal (e.g. 3036 in FIG. 52C). In some examples, user interface 3040 comprises a user interface or other display that provides for the simultaneous display, activation, and/or operation of at least some of the sensors, sensing element, respiration determination elements, stimulation elements, power/control elements (e.g. pulse generators), devices, user interfaces, instructions, information, engines, functions, actions, and/or method, etc., as described in association with FIG. 1-52B and FIG. 52E-101. In some examples, at least some portions or aspects of the user interface 3040 are provided via a graphical user interface (GUI), and may comprise a display 3044 and input 3042.

FIG. 52E is a block diagram 3050 which schematically represents some example implementations by which an implantable device (IMD) 3060 (e.g. 283 in FIG. 2B, 2833 in FIGS. 50-51, 2833 in FIG. 50, 100-101, 2819B in FIG. 51, 102), implantable sensing monitor, and the like) may communicate wirelessly with external devices outside the patient. As shown in FIG. 52E, in some examples, the IMD 3060 may communicate with at least one of patient app 3072 on a mobile device 3070, a patient remote control 3074, a clinician programmer 3076, and a patient management tool 3080. The patient management tool 3080 may be implemented via a cloud-based portal 3082, the patient app 3072, and/or the patient remote control 3074. Among other types of data, these communication arrangements enable the IMD 3060 to communicate, display, manage, etc. the AHI determination information, ODI determination information, as well as to allow for adjustment to the various elements, portions, etc. of the example devices and methods if and where desired. In some examples, the various forms of identified sleep disordered breathing (e.g. AHI, ODI) may be displayed to a patient and/or clinician via one of the above-described external devices. The displayed information may comprise each event of sleep disordered breathing, a nightly aggregate of such events, or trends regarding such sleep disordered breathing.

As previously noted, determining sleep disordered breathing and/or treating sleep disordered breathing provides just one example of managing disease for a patient, such that a sleep disordered breathing indicator (e.g. AHI) may comprise just one example of a disease burden indicator.

FIG. 53A is block diagram schematically representing an example method 4000 and/or example device for construction of a data model 4057 to determine a disease burden indicator. In some examples, method 4000 (or example device) may comprise one example implementation of, and/or may comprise at least some of substantially the same features and attributes, as the previously described constructable and constructed data models in association with at least FIG. 4-12B.

As shown in FIG. 53A, an example method (and/or example device) comprising providing known inputs 4020 and known outputs 4070 to form or construct a data model 4057 (i.e. constructable data model 4057). As further described later, the known inputs 4020 may be provided via implanted sensor(s) or other implanted elements. In some such examples, the known inputs 4020 may exclude externally sensed or provided inputs.

In some examples, the known inputs 4020 may comprise inputs obtained via external sensors (or other external elements) in addition to the known inputs sensed or obtained via implanted sensors (and/or other implanted elements).

In some examples, the known inputs 4020 may comprise solely externally sensed or obtained inputs without any implanted sensors or other implanted elements.

As shown in FIG. 53A, in some examples the known output 4070 may comprise a disease burden indicator 4040 and/or a measurable physiologic parameter 4072, either of which may be externally measurable in at least some examples. Further details regarding the known output 4070 will be described later. As previously described in association with at least FIG. 8-12B, constructing the data model may comprise training a data model, such as one of the data models in data model types 600 in FIG. 10A with one of the example data model types comprising a machine learning model 602.

As further shown in FIG. 53A, in some examples at least some known inputs 4020 regarding the patient’s body comprise a respiratory rate 4022, patient activity 4024, patient motion 4025, posture 4027, and/or seismocardiography 4028. In some such examples, these known inputs are obtained via implanted sensor(s), which in some examples may comprise an accelerometer. In some such examples, known inputs 4020 obtainable from an implanted accelerometer (and/or other types of sensors, elements, etc.) may comprise at least some of substantially the same features and attributes as described in association with at least FIGS. 2B-3C and FIG. 56A-102.

In some examples, a known input 4020 may comprise a sleep-wake status 4029 of the patient. In some examples, the sleep-wake status may be sensed or obtained via an accelerometer or via other elements which may identify activity, heart rate, respiratory patterns, posture, motion, etc. indicative of a sleep-wake status. In some examples, with regard to determining a sleep-wake status, at least some such sensors, elements, and/or the accelerometer may be implantable, while in some examples, at least some such sensors, elements, and/or accelerometer may be external of the patient’s body.

In some examples, the known inputs 4020 may be provided via implanted sensors (e.g. electrodes) and/or elements other than an accelerometer. Accordingly, in some examples, some known inputs 4020 may comprise an implanted electrocardiograph sensing (ECG) 4030, implanted electroencephalograph sensing (EEG) 4032, and/or implanted bio-impedance sensing 4034. In some such examples, the respective sensors may comprise sensor electrodes which are spaced apart from each other across a portion of patients’ body, such as thoracic region, head-and-neck region, other patient body regions, and combinations thereof.

In some examples, the known inputs 4020 may be provided via external sensor(s) and/or external elements. At least some such example known inputs 4020 may comprise an external ECG 4035, a lung fluid volume (e.g. via chest imaging) 4036, an external oxygen saturation (or desaturation) 4037, and/or a cardiac catheterization 4038. In some examples, the oxygen saturation (or desaturation) 4037 may be obtained via pulse oximetry, such as may be obtainable externally via a finger or other body portion. In some examples, the cardiac catheterization 4038 may comprise sensors deliverable to and within a cardiac region to sense cardiac information, such as but not limited to cardiac waveforms.

In some examples, the known inputs 4020 may comprise an external accelerometer 4038. In some such examples, the external accelerometer 4038 may be used to sense body motion or activity, which may comprise shaking, tremors, irregular body/muscle movements, and the like.

It will be understood that any single or combination of the various known inputs 4020 may be used as known inputs in forming the constructable data model 4057. In some examples, just one or some of the known inputs 4020 may be used to construct a data model, while in some examples all of the known inputs 4020 may be used to construct a data model. Moreover, with respect to the arrangements in FIGS. 53A-55C, just one known input 4020 or just some of the known inputs 4020 may be applicable to a particular disease burden indicator.

With further reference to at least FIG. 53A, while some known inputs 4020 are labeled as being sensed via an implanted accelerometer, it will be understood that in some examples, some such physiologic information may be sensed via implantable sensors other than (and/or in addition to) an accelerometer. In addition, while some known inputs 4020 are not labeled as being sensed via an implanted accelerometer, it will be understood that in some examples, some such physiologic information may be sensed via an implanted accelerometer instead of, or in addition, to being sensed via sensors other than an accelerometer

Meanwhile, the known output 4070 used to in constructing a data model (FIG. 53A) may comprise at least some measurable physiologic parameters 4072, which generally may comprise externally measurable parameters in some examples. In some such examples, the externally measurable physiologic parameters may comprise polysomnography(PSG)-type parameters, which may be obtained in a formal sleep study venue or may be obtained informally in the home or elsewhere.

In some examples, the measurable physiologic parameter 4072 may be associated with a disease burden indicator 4040, such that measurement of the physiologic parameter 4072 may produce data suitable to determine the disease burden indicator 4040.

However, in some examples the disease burden indicator 4040 may act as a known output 4070 independent of at least some measurable physiologic parameter(s) 4072.

With further reference to FIG. 53A, in some examples the disease burden indicator 4040 may be expressed as quantitative value, and may comprise an index, rating, etc. regarding the particular disease. In some examples, the disease burden indicator 4040 may be expressed with regard to a reference value, threshold, criteria, etc. or without regard to a reference value, threshold, criteria, etc. In some examples, the disease burden indicator 4040 may be expressed with regard to changes (e.g. increase, decrease, no change) relative to a baseline value of disease burden or without regard to a baseline value of disease burden.

As further shown in FIG. 53A, in some examples, the disease burden indicator 4040 may comprise a class parameter 4042 and/or a trend parameter 4044. The class parameter 4042 may express the disease burden indicator 4040 in terms of classes, such as but not limited to, classes according to intensity or severity. Meanwhile, the trend parameter 4044 may express the disease burden indicator 4040 in terms of trends, such as if or when a parameter of the disease burden indicator 4040 increases over time or decreases over time, such as monitoring period (e.g. 4091 in FIG. 53C). In some such examples, such increases and/or decreases may be in response to application of therapy or in the absence of therapy.

In some examples the class parameter 4042 may be at least partially implemented according to a class arrangement 4080, as shown in FIG. 53B, by which different levels of disease burden may be assigned to different classes. In some examples, the different levels of disease burden may be indicated (i.e. a disease burden indicator) according to quantitative values (e.g. QV1, QV2, etc.), which also may comprise ranges of quantitative values in some examples. As further shown in FIG. 53B, the class arrangement 4080 may comprise different classes of disease burden indication as expressed via the different rows 4081, and a column 4082 representing a label (e.g. A, B, C, etc.) to identify the different classes.

As further shown in FIG. 53B, in some examples one example column 4085 of the class arrangement 4080 may represent different quantitative values (or different ranges of quantitative values) of disease burden indication, with indicators QV1, QV2, etc. in FIG. 53B representing such different quantitative values. In some such examples, the different classes of disease burden indication may be expressed relative to reference. In some examples, the reference may comprise an index, rating, threshold, etc.

For instance, in examples in which the disease burden indicator may comprise a sleep disordered breathing (SDB) indicator, the reference may comprise an apnea-hypopnea index (AHI) and the quantitative values may be arranged into the following classes: (A) AHI<5; (B) AHI<15); (C) AHI< 30; (D) AHI>30. In some such examples, the different classes also may be assigned qualitative values as represented in column 4083. For instance, in examples in which the disease burden indicator comprises a sleep disordered breathing (SDB) indicator, such as but not limited to an AHI, the qualitative values may comprise Normal (AHI<5), Low (AHI<15), Moderate (AHI<30), and High (AHI>30). However, it will be understood that a wide variety of different expressions may be used to represent different classes of qualitative values.

With further reference to FIG. 53A, in some examples the trend parameter 4044 may be at least partially implemented according to a trend arrangement 4090, as shown in FIG. 53C, by which changes in a parameter (e.g. quantitative value) of a disease burden indicator over a time period may be tracked and expressed as a trend or other pattern. In some such examples, the time period may sometimes be referred to as a monitoring period 4091. By monitoring such changes over time, the example methods and/or example devices may facilitate assessing disease burden because these changes over time present opportunities for clinical intervention to improve outcomes or for concluding that a clinical intervention was successful.

As further shown in FIG. 53C, the trend arrangement 4090 may comprise a burden parameter 4092 by which increases (I) and/or decreases (D) in disease burden may be indicated generally, i.e. is the disease getting better or worse ? In some examples, the trend arrangement 4090 may comprise an inverse parameter 4094 by which quantitative values of the disease burden indicator may have an inverse relationship with the actual state (e.g. increasing or decreasing) of the disease burden.

In some examples, an increase in the disease burden indicator over the monitoring time period (e.g. a trend) corresponds to an increase in the actual disease burden while a decrease in the disease burden indicator (over the monitoring time period) corresponds to a decrease in the disease burden. Given the above-example trend, one example response may comprise concluding that the increase in the disease burden indicator suggests that it may be beneficial to: make increases in therapy intensity; and/or implement a different therapeutic intervention, such as a more aggressive therapeutic intervention. Another example response may comprise concluding that the decrease in the parameter of the disease burden indicator suggests that it may be beneficial to: make decreases in therapy intensity; and/or implement a different therapeutic intervention, such as a less aggressive therapeutic intervention.

However, in some examples in which quantitative values of the disease burden indicator may have an inverse relationship (per inverse parameter 4094) with the actual state (e.g. increasing or decreasing) of the disease burden, an increase in disease burden may be expressed via lower quantitative values. In some such examples, one example response may comprise concluding that the decrease in the quantitative values of the disease burden indicator suggests that it may be beneficial to: make increases in therapy intensity; and/or implement a different therapeutic intervention, such as a more aggressive therapeutic intervention. Another example response may comprise concluding that the increase in the quantitative values of the disease burden indicator suggests that it may be beneficial to: make decreases in therapy intensity; and/or implement a different therapeutic intervention, such as a less aggressive therapeutic intervention.

In some examples, a magnitude of change (e.g. small or large) in a disease burden indicator may be indicative of whether a change in therapy intensity (or use of a different type of therapy) may be beneficial. The magnitude of change also may be considered in relation to the time period (e.g. 4091) over which the change takes place, which may be indicative of the relative stability of the disease burden indicator. For instance, in some examples, the particular change in a magnitude of the disease burden indicator may occur over a long time period such that the change is considered gradual and a long term change, such that an abrupt change in therapy may be undesirable. In some examples, the particular change in the disease burden indicator may occur over a short time period such that the change may be viewed as a short term shift, which may be temporary, rather than a long term change. On the other hand, a change of high magnitude in a short time period sometimes may indicate that intervention is warranted, depending on the particular type or state of disease.

In some examples, at least some features and attributes of the class arrangement 4080 and/or trend arrangement 4090 may be displayable via a user interface (e.g. in FIG. 52D) and/or associated devices (FIG. 52E) to facilitate observing the patient’s health according to the different classes or trends of disease burden (indicator) and the particular disease burden indicator applicable for the patient in real-time or at different historical points in time.

Depending on the particular class (e.g. per class arrangement 4080) and/or trend (e.g. per arrangement 4090) identified via the disease burden indicator, an example method and/or example device may further comprise applying therapy to treat the disease burden. In some such examples in which the disease burden indicator may comprise a sleep disordered breathing (SDB) indicator, therapy may comprise applying nerve or muscle stimulation to treat the disease, such as obstructive sleep apnea, central sleep apnea, or multiple-type sleep apnea.

In some examples, the different classes of disease burden indication (e.g. per class parameter 4042, arrangement 4080) and/or trend information (e.g. per trend parameter 4044, arrangement 4090) may be used as known outputs 4070 in constructing a data model 4057 as described in association with FIG. 53A. Accordingly, the construction of data model 4057 may implemented according to the types and ways in which a clinician may utilize the disease burden indication in diagnosing, monitoring, treating, etc. the patient.

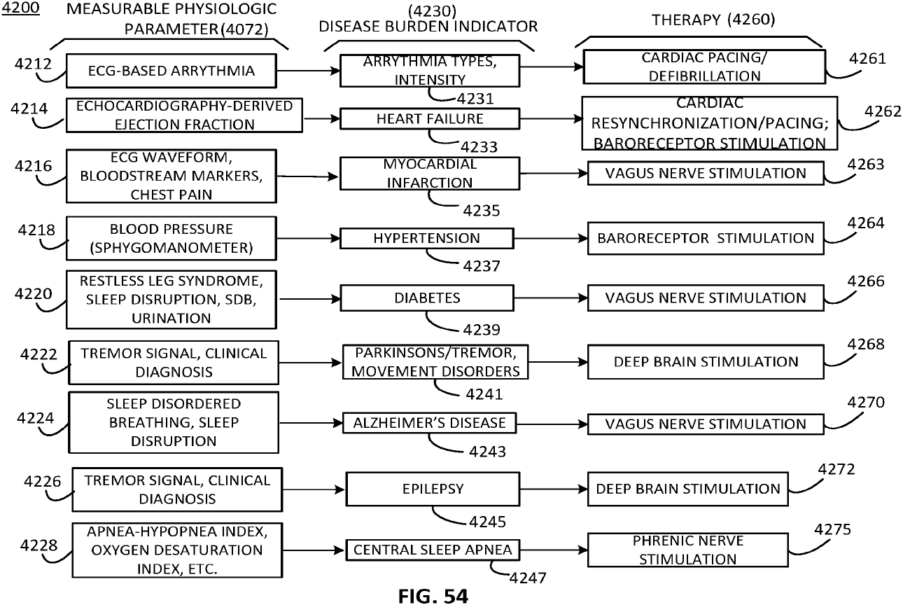

By providing such known inputs (4020) and known outputs (4070) to the constructable data model 4057, a constructed data model 4123 (FIG. 54) may be obtained. As noted elsewhere, the constructable data model 4057 (FIG. 53A) may comprise a trainable machine learning model and the constructed data model 4123 (FIG. 54) may comprise a trained machine learning model (e.g. 602 in FIG. 10A).

FIG. 54 is a block diagram 4200 schematically representing some known outputs 4070 (FIG. 53A) for use in constructing a data model (e.g. FIG. 53A) which may be expressed as a given measurable physiologic parameter 4072 (FIG. 53A). FIG. 54 also schematically represents a relationship between at least some of those measurable physiologic parameters 4072, a given disease burden indicator 4230, and/or a therapy 4260.

In some examples, the known output 4072 may be expressable as a measurable physiologic parameter (e.g. 4072). In some examples, the measurable physiologic parameter may comprise an ECG-based arrhythmia 4212 (such as may be obtained via an external ECG), which in turn may provide a disease burden indication 4230 expressed as an arrhythmia indication 4231, such as specific types of cardiac arrhythmia, intensity of cardiac arrhythmia, etc. At least some specific types of cardiac arrhythmia may comprise atrial fibrillation, ventricular fibrillation, ventricular tachycardia, bradycardia, etc. In some examples, a corresponding therapy for this arrhythmia indication 4231 may comprise cardiac therapy 4261, such as but not limited to cardiac pacing, cardiac defibrillation, and the like.

In some examples, the measurable physiologic parameter (e.g. known output 4072) comprises an ejection fraction 4214 (such as derived from echocardiography), which in turn may provide a disease burden indication 4230 expressed as an indication of heart failure 4233 (e.g. congestive heart failure). In some examples, a corresponding therapy for this heart failure indication 4233 may comprise therapy 4262, such as but not limited to cardiac pacing, baroreceptor (e.g. carotid sinus) stimulation, and the like.

In some examples, the measurable physiologic parameter (e.g. known output 4072) comprises one or more parameters 4216 (e.g. ECG waveform, bloodstream cardiac markers, chest pain, etc.), which in turn may provide a disease burden indication 4230 expressed as an indication of myocardial infarction 4235. In some examples, a corresponding therapy for this myocardial infarction indication 4235 may comprise therapy 4263, such as but not limited to vagus nerve stimulation, and the like.

In some examples, the measurable physiologic parameter (e.g. known output 4072) comprises a blood pressure (such as obtained via a sphygmomanometer), which in turn may provide a disease burden indication 4230 expressed as an indication of hypertension 4237. In some examples, a corresponding therapy for this hypertension indication 4237 may comprise therapy 4264, such as but not limited to baroreceptor (e.g. carotid sinus stimulation), and the like.

In some examples, the measurable physiologic parameter (e.g. known output 4072) comprises parameters 4220 including restless leg syndrome, sleep disruption, sleep disordered breathing, and/or frequent urination. These parameters may, in turn, provide a disease burden indication 4230 expressed as a diabetes indication 4239. In some examples, a corresponding therapy for this diabetes indication 4239 may comprise therapy 4266, such as but not limited to vagus nerve stimulation, and the like. With further reference to the diabetes indication 4239 as one example disease burden indicator, diabetes provides one non-limiting example of identifying disease from the reference point of sleep. For example, some patients a diagnosis of diabetes may include a presenting symptom or behavior which occurs during sleep, such as but not limited to a lack of sleep quality (e.g. sleep disruption). In some such examples, the sleep disruption may be due to restless leg syndrome, which is detectable at least via motion, activity, etc. sensed via an accelerometer. The restless leg syndrome, in turn, may result from neuropathic pain, which in turn may trace its roots from diabetes.

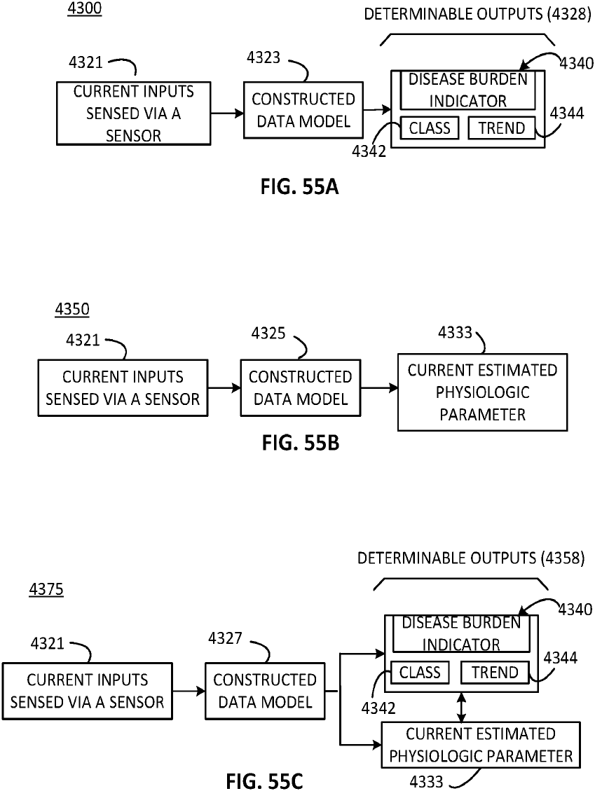

With this in mind, at least some these measurable physiologic parameters 4220 (FIG. 54) may be used as known outputs 4072 in constructing a data model 4057 (FIG. 53A) which becomes trained (i.e. constructed) to sense internally sensed (e.g. via implantable sensors) physiologic parameters (e.g. motion, activity, etc.) in order to provide a diabetes disease burden indication 4239 upon current inputs (e.g. 4321 in FIGS. 55A-55C) being fed into a constructed data model (e.g. 4323 in FIG. 55A; 4325 in FIG. 55B; 4327 in FIG. 55C). In some examples, the current inputs 4321 may comprise at least some of the inputs 4020 in FIG. 53A when currently sensed. As previously noted, in some such examples at least some of the inputs 4020 may be sensed via an implanted accelerometer(s).

With further reference to FIG. 54, in some examples, the measurable physiologic parameter (e.g. known output 4072) comprises parameters 4222 (e.g. tremor signal, clinical diagnosis, etc.), which in turn may provide a disease burden indication 4230 expressed as an indication 4241 of diseases involving tremors or irregular bodily movements (e.g. Parkinson’s, movement disorders, etc.). The movement disorders may comprise dystonia, myoclonus, ALS, and the like. In some examples, a corresponding therapy for this disease burden indication 4241 may comprise therapy 4268, such as but not limited to deep brain stimulation, and the like.

In some examples, the measurable physiologic parameter (e.g. known output 4072) comprises parameter 4224 relating to sleep disordered breathing, sleep disruption, and the like. In some such examples, an apnea-hypopnea index (AHI), oxygen desaturation index (ODI), arousal parameter, or similar indicators may act as an externally measurable physiologic parameter indicative of sleep disordered breathing and/or sleep disruption (e.g. lack of sleep quality). These physiologic parameters 4220 may, in turn, provide a disease burden indication 4230 expressed as Alzheimer’s disease 4243. In some examples, a corresponding therapy for this Alzheimer’s disease indication 4243 may comprise therapy, such as but not limited to vagus nerve stimulation 4270, and the like.

In some examples, the measurable physiologic parameter (e.g. known output 4072) comprises parameter 4226 (e.g. clinical diagnosis, etc.), which in turn may provide a disease burden indication 4230 expressed as an epilepsy indication 4245. In some examples, a corresponding therapy for this epilepsy indication 4245 may comprise therapy, such as but not limited to deep brain stimulation 4272, and the like. With regard to constructing a data model (e.g. 4057 in FIG. 53A) to identify an epilepsy indication 4245, a further known input 4020 may comprise an EEG obtained via an external sensor.

In some examples, the measurable physiologic parameter (e.g. known output 4072) comprises parameter 4228 (e.g. apnea-hypopnea index (AHI), oxygen desaturation index (ODI) etc.), which in turn may provide a disease burden indication 4230 expressed as a central sleep apnea indication 4247. In some examples, a corresponding therapy for this central sleep apnea indication 4247 may comprise therapy, such as but not limited to phrenic nerve stimulation 4275, and the like. With regard to constructing a data model (e.g. 4057 in FIG. 53A) to identify a central sleep apnea indication 4247, a further known input 4020 may comprise externally-sensed EEG, respiratory effort, nasal pressure, and the like.

With reference to FIGS. 53A-55C, it will be understood that in some examples not all of the listed inputs (e.g. 4020 in FIG. 53A) may be applicable to each listed disease burden indicator (e.g. 4230 in FIG. 54) or vice versa. It will be further understood that the listed known inputs (e.g. 4020 in FIG. 53A) are not an exhaustive list of known inputs which may help determine any single disease burden indicator (e.g. 4230 in FIGS. 54, 55A, 55C) and that the listed disease burden indicators (e.g. 4230 in FIG. 54) are not an exhaustive list of disease burden indicators which may be determined from one or more of the particular inputs 4020 in FIG. 53A. Similarly, in some examples, the list of measurable physiologic parameters 4072 in FIG. 54 may not comprise an exhaustive list of known outputs (e.g. 4070 in FIG. 53A) when constructing a data model.

It will be further understood with regard to both the construction of data model 4057 in FIG. 53A and use of a constructed data model (e.g. 4323, 4325, 4327) in FIGS. 55A-55C, the known output 4070 in FIG. 53A and the determinable output 4328 in FIG. 55A may comprise a disease burden indicator 4340 (FIG. 55A), a current estimated physiologic parameter 4333 (FIG. 55B), or both a disease burden indicator 4340 and current estimated physiologic parameter 4333 (FIG. 55B).

FIG. 55A is a diagram schematically representing an example method 4300 (and/or device) of using a constructed data model 4323 for determining a current disease burden indicator 4340. As shown in FIG. 55A, currently sensed inputs 4321 are fed into the constructed data model 4323 (e.g. trained machine learning model), which then produces a determinable output 4328, such as a current disease burden indicator 4340, which is based on the current inputs 4020. In some examples, the current inputs 4321 are obtained via an implanted accelerometer (e.g. 285 in FIG. 2, 304A, 322A in FIGS. 2B-3B) and the current inputs 4021 correspond to the types of known inputs 4020 (e.g. 4022, 4024, 4026 in FIG. 53A) obtained via the implanted accelerometer. However, in some examples, the current inputs 4321 in FIG. 55A may comprise all or just some of the inputs 4020 (FIG. 53A), whether the inputs are sensed via an accelerometer and/or via other types of sensors, elements, etc.

It will be understood that when employing data model 4323 in FIG. 55A to determine a current disease burden indicator 4340), in some examples the current inputs 4321 omit (i.e. do not include) any externally measurable known inputs 4020 (FIG. 53A) which may have been used in constructing the data model (FIG. 53A). However, in some examples, the current inputs 4321 in FIG. 55A may sometimes include some externally measurable inputs.

In some examples, the constructed data model 4323 in FIG. 55A may be constructed according to the example methods and/or devices as previously described in association with at least FIG. 53A-54 and FIG. 8-12B. Of course, it will be understood that the principles of at least some of the examples in association with FIGS. 53A-55C may be applicable to other examples of the present disclosure, such as examples in which the disease burden indicators comprises sleep disordered breathing, blood oxygenation, etc.

FIG. 55B is a diagram schematically representing an example method 4350 (and/or device) comprising at least some of substantially the same features and attributes as example method 4300 (and/or device), except with the determinable output 4328 comprising a current estimated physiologic parameter 4333.

FIG. 55C is a diagram schematically representing an example method 4375 (and/or device) comprising at least some of substantially the same features and attributes as example methods 4300, 4350 (and/or device), except with the determinable output 4328 comprising a disease burden indicator 4340 and/or a current estimated physiologic parameter 4333.

With respect to at least FIGS. 53A-55C and as previously noted elsewhere, by obtaining a current estimated physiologic parameter 4333 and/or current disease burden indicator 4340 via a constructed data model (4323, 4325, 4327), in some examples the example methods and/or example devices may provide the desired information without the use of external sensors for inputs or determining outputs. In some such examples, the use of implantable sensors (e.g. accelerometer, etc.) may enable more efficient and/or effective diagnosis, monitoring, treatment of various diseases, conditions, etc. in association with an implantable medical device.

FIG. 56A-102, and their accompanying description, provide further details regarding examples of respiratory detection, such as but not limited to via an implantable accelerometer. This respiratory detection may be employed to identify and/or treat diseases, such as but not limited to sleep disordered breathing. Moreover, this respiratory detection may be employed to provide known inputs in constructing and/or using a constructed data model to determine disease burden indication (or current estimated physiologic parameter) according to at least some of the various examples of the present disclosure.

Accordingly, with further reference to at least FIGS. 3A-3C, FIGS. 56A, 56B, 56C are diagrams which schematically represent an example method and/or example sensor 5004 which may comprise three sensing elements 322A (Y), 5062 (Z), 5064 (X) arranged orthogonally relative to each other. In some examples, the sensor 5004 (including at least sensing element 322A) comprises at least some of substantially the same features and attributes as sensor 304A previously described in association with at least FIGS. 3A-3B in which just one sensing element 322A (Y) is present. However, as shown in FIGS. 56A-56B, in addition to sensing element 322A (Y), in some examples sensor 304A also may comprise acceleration sensing element 5062 having orientation Z (Z-axis) which is perpendicular to sensing element 322A. As implanted, this Z-axis orientation is generally perpendicular to a superior-inferior (S - I) orientation of the chest wall 302A, and is generally parallel to an anterior-posterior (A-P) orientation of the chest wall 302A.

Meanwhile, as shown in FIG. 56B, in addition to comprising sensing element 322A (Y), in some examples sensor 304A also may comprise acceleration sensing element 5064, having orientation X (X-axis) which is generally perpendicular to sensing element 322A. As implanted, this X-axis orientation is generally perpendicular to a superior-inferior (S - I) orientation of the chest wall 302A, and generally perpendicular to an anterior-posterior (A-P) orientation of the chest wall 302A. In some such examples, sensing element 5064 may sense rotational movement of chest wall 302A (as represented by directional arrow B5) in a plane defined by the anterior-posterior orientation (A-P) and by the lateral-medial orientation (L-M), according to changes in an inclination angle (as represented via directional arrow B4) of sensing element 5064. Each of the respective sensing elements 5062 (Z), 5064 (X) may provide additional sensing of rotational movement of the chest wall 302A to provide further respiration information.

With further reference to FIGS. 56A-56C, in some examples, sensor 5004 may comprise all three sensing elements 322A (Y), 5062 (Z) and 5064 (X).

As schematically represented in the diagram 5250 of FIG. 56C, the sensed acceleration signal information from each of the three sensing elements 5062, 322A, 5064 of sensor 5004 may be combined to provide composite rotational change information (5252). In some examples, the composite rotational change information 5252 may sometimes be referred to as a virtual vector representing the overall rotational movement (e.g. according to at least two orthogonal axes) caused by breathing. In some such examples, the composite rotational change information 5252 corresponds to sensing the AC component of the multi-dimensional acceleration vector (e.g. a virtual vector) with respect to gravity.

In some examples, at least two of the three orthogonally-arranged sensing elements may be used to perform determination of composite rotational movement and therefore respiration information at least based on an AC component of a multi-dimensional acceleration vector produced by the n single-axis sensing elements.

In some such examples associated with FIG. 56C, the virtual vector corresponding to the composite rotational change (5252) may exhibit higher sensitivity to respiration than any single vector of a physical sensing element 322A (Y), sensing element 5062 (Z), or sensing element 5064 (X). In some such examples, the virtual vector (5252) may exhibit a higher signal-to-noise ratio (e.g. signal quality) than any single physical vector, such as single sensing element 322A (Y) or single sensing element 5062 (Z) or single sensing element 5064 (X) by virtue of combining the signals of the multiple sensing elements.

In some such examples, the virtual vector (e.g. 5252) effectively excludes non-physiologic motion of the chest wall. At least some examples of such non-physiologic motion may comprise motion of a vehicle (e.g. car, airplane, etc.) within which the patient is riding, of patient swinging in a hammock, and the like. Accordingly, determining respiration information via the virtual vector in such example methods and/or devices may produce respiration information which is generally insensitive to non-physiologic motion of the patient.

In some examples, respiration detection may be based on a sum of two of the vectors from among the three orthogonally-arranged sensing elements 322A, 5062, 5064 in FIG. 56C. In some examples, respiration detection may be based on a sum of signals from all three orthogonally-arranged sensing elements 322A, 5062, 5064 in FIG. 56C.

In some examples, respiration detection may be determined by looking independently at each of the three vectors (e.g. 322A, 5062, 5064) or from among the three vectors.

In some examples, a method and/or device may employ control portion 3000 (FIG. 52B) to select the virtual vector (e.g. 5252) or a physical vector from one of the sensing elements 322A, 5062, or 5064 for use in determine respiration information. In some such examples, the method and/or device may evaluate the robustness of the determined respiration information and automatically convert operation among the virtual vector (e.g. 5252 in FIG. 56C) and any one of the physical vectors (e.g. 322A/Y, 5062/Z, 5064/X) to consistently use the most robust, accurate signal source in determining respiration information.

In association with the examples of at least FIGS. 56A, 56B, 56C, in some examples the signal-to-noise ratio of a virtual vector and/or physical vector may be enhanced via excluding noise, such as later described in association with at least noise model parameter 7470 (FIG. 75D), method 7885 (FIG. 85), and/or method 7890 (FIG. 86).

In some examples, the above-described measuring of rotational movement (of a portion of a chest wall via acceleration sensing) per sensing element 5062 (Z-axis) may be likened to a pitch parameter, measuring rotational movement per sensing element 322A (Y-axis) may be likened to a yaw parameter, and measuring rotational movement per sensing element 5064 (X-axis) may be likened to a roll parameter. Because of variances in anatomy from patient to patient, the particular implant orientation, and/or the particular implant location (e.g. front vs. side of the chest), the pitch parameter, yaw parameter, and/or roll parameter may bear a rough or general correspondence to the ideal definition for such respective parameters in which the pitch parameter may correspond to rotational movement of the portion of the chest wall in a first plane defined by an anterior-posterior orientation and by a superior-inferior orientation of the patient’s body. Similarly, the yaw parameter may roughly or generally correspond to rotational movement of the portion of the chest wall in a second plane defined by the anterior-posterior orientation and by a lateral-medial orientation of the patients’ body. Similarly, the roll parameter may roughly or generally correspond to rotational movement of the portion of the chest wall in a third plane defined by the lateral-medial orientation and by the superior-inferior orientation of the patient’s body.

In examples in which the patient’s body position corresponds to a primary sleeping position (e.g. generally horizontal), then the magnitude of changes in the AC signal component from rotational movement (B3) sensing element 5062 (Z axis) during breathing will be negligible and the magnitude of changes in the AC signal component from rotation (arrow B4) of sensing element 5064 (X axis) during breathing may be relatively small at least compared the magnitude of changes in the AC signal component of sensing element 322A (Y-axis) during breathing (as described in association with FIGS. 3A-3C).

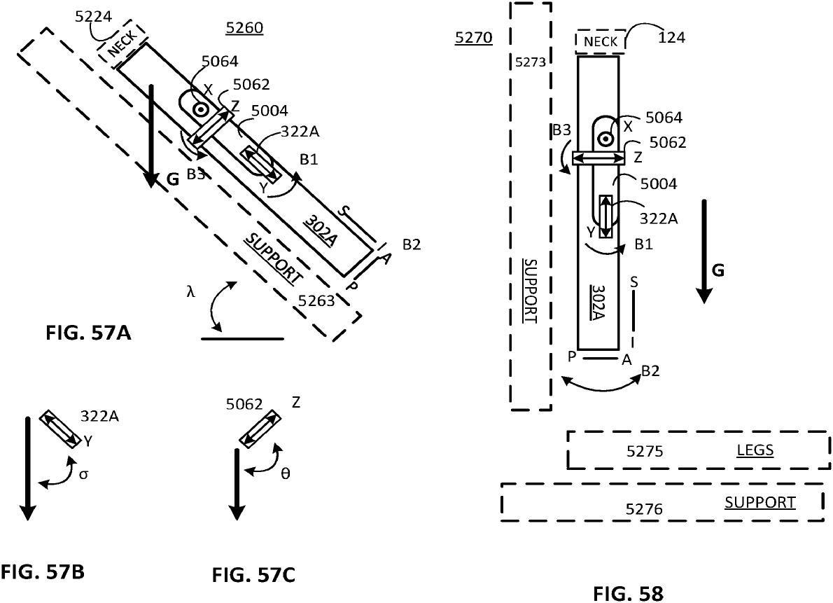

However, in some example situations the patient’s body position may correspond to a secondary or alternate sleep position, such as sitting upright against a support 5273 (e.g. ordinary chair, airplane chair, etc.) as shown in FIG. 58 or in a partially reclined position (e.g. torso is 45 degrees from horizontal) against a support 5263 (e.g. recliner chair, recliner bed, etc.) which is at angle (λ) relative to generally horizontal (e.g. floor) as shown in FIG. 57A. In some such examples, such as the partially reclined position in FIG. 57A, at least the respective sensing element 5062 (Z axis) may yield significant magnitude of changes in the AC signal component during breathing instead of and/or in addition to sensed changes in the AC signal component of sensing element 322A (Y-axis) during breathing. In this example, the sensing element 322A may comprise a first angular orientation (like YR1 in FIG. 3C for peak expiration) which is 45 degrees (σ in FIG. 57B) relative to the gravity vector G (and which is 45 degrees relative to a generally horizontal plane, which typically is a primary sleep position). While the first orientation (e.g. YR1) of the sensing element 322A may not be generally perpendicular to the gravity vector G as in FIGS. 3A-3B, at the first orientation of 45 degrees (σ in FIG. 57B) relative to the gravity vector G, the acceleration sensing element 322A still exhibits sufficient sensitivity in the AC signal component to produce meaningful measurements in changes of the inclination angle (e.g. Ω in FIG. 3B) of sensing element 322A between the first and second orientations (e.g. YR1 and YR2) during breathing to enable determining respiration information.

In this example, the sensing element 5062 may comprise a first orientation (like YR1 in FIG. 3B) which extends at an angle of 135 degrees (θ in FIG. 57C) relative to the gravity vector G (and which is 45 degrees relative to a generally horizontal plane, which typically is a primary sleep position). While the sensing element 5062 may not be generally perpendicular to the gravity vector G (as was sensing element 322A in the example of FIG. 3B), at the first orientation of 135 degrees (θ in FIG. 57C) relative to the gravity vector G, the acceleration sensing element 5062 exhibits sufficient sensitivity in the AC signal component to produce meaningful measurements in changes of the inclination angle (like Ω in FIG. 3B) of sensing element 5062 between its first orientation (peak expiration) and second orientation (peak inspiration) during breathing to enable determining respiration information.

In a manner similar to that shown in FIG. 56C, the sensed rotational movement from at least the multiple sensing elements (e.g. 5062/Z-axis and 322A/Y-axis in FIGS. 57A-57C) may be combined to yield a composite value of sensed rotational movement of sensor 5004 in order to produce sensing of a respiratory waveform while the patient is in the partially reclined position.

It will be further understood that, in some examples, the sensing element 5064 (X-axis) also may be used in addition to sensing elements 322A, 5062 (and in a manner similar to that described for sensing elements 322A, 5062 in FIGS. 57A-57C) to provide further sensing by which the determination of respiration information can be made, with the rotational sensing information being combined, similar to that shown in FIG. 56C. With this in mind and with further reference to at least the examples of FIGS. 57A-57C and 58, it will be understood that employing a three axis accelerometer (in which the three axes are orthogonally-arranged) will ensure that at least one of the three axes will have an output signal of magnitude sufficient to reliably determine respiration (e.g. based on rotational movement of the sensor in correspondence with rotational movement of a portion of the chest wall during breathing as described in various examples).

It will be understood that in some examples, the particular angle λ of reclination in FIG. 57A may be angles other than 45 degrees, and may be variable over time in some instances, depending on the type and manner of support 5263 (e.g. adjustable bed, chair). In some such examples, a determination of respiration information may be based on the particular respective sensing element(s) (e.g. 322A (Y-axis), 5062 (Z-axis), 5064 (X-axis)) having the orientation(s) closest to being generally perpendicular to the gravity vector G for the particular angle λ at a particular point in time.

Moreover, in this example arrangement of FIG. 57A-58, if and/or when the patient moves to another sleeping position, such as generally horizontal position (e.g. FIGS. 3A-3B), then the sensing element 322A (or sensing element 5064) may become the sole or primary signal source for detecting respiration in some examples.

Accordingly, example arrangements of multiple single-axis acceleration sensing elements in orthogonal relationship to each other may provide robust sensing of respiration which enables adaptability in response to a patient moving among different sleep positions within a single treatment period or among multiple, different treatment periods.

As previously noted, FIG. 58 schematic represents at least a chest wall 302A of a patient’s body in a generally vertically upright position, such as if the patient were sitting on a support 5276 with their torso against a vertical support 5273. In this example arrangement, both the acceleration sensing elements 5062 (Z-axis) and 5064 (X-axis) of sensor 5004 may have a first orientation which is generally perpendicular (or reasonably close to being generally perpendicular) to gravity vector G, whereas the acceleration sensing element 322A (Y-axis) of sensor 5004 has a general orientation which is generally parallel to gravity vector G. Accordingly, for substantially similar reasons presented with respect to at least FIGS. 3A-3B and 56A-57C, one or both of the sensing elements 5062 (Z-axis), 5064 (X-axis) may provide the most sensitive sensing elements by which respiration information determination may be performed. In particular, upon rotational movement of the patient’s chest wall 302A during breathing within a treatment period, rotational movement of Z-axis sensing element 5062 between a first orientation (e.g. like YR1 in FIG. 3B) and a second orientation (e.g. like YR2 in FIG. 3B) may be sensed as range of values of an AC signal component from which a respiratory waveform (including respiratory phase timing/details) may be determined as shown in FIG. 3C. Moreover, rotational movement of X-axis sensing element 5064 may provide similar information and may be used to determine respiration information. The respiration information may be determined solely from the Z-axis sensing element 5062, solely from the X-axis sensing element 5064, or from a combination of information sensed via both of the Z-axis sensing element 5062 and the X-axis sensing element 5064. While the Y-axis sensing element 322A would generally be expected to produce negligible or minimal respiration information (because of being parallel to the gravity vector G), in some examples, information sensed from Y-axis sensing element 322A may be combined with rotational information sensed via the sensing elements 5062, 5064.

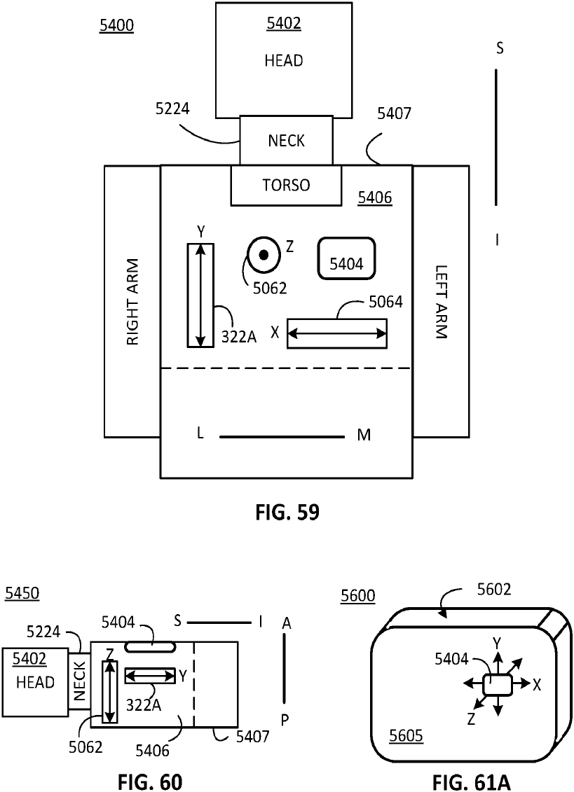

FIG. 59 is a diagram 5400 including a front view schematically representing different measurement axes of an example sensor 5404 and/or related example method. In some examples, the sensor 5404 may comprise at least some of substantially the same features and attributes as the sensors, sensing elements, and related example methods as previously described in association with FIG. 3A-58. As shown in FIG. 59, sensor 5404 is implanted within a wall of chest region 5406 of torso 5407 below a neck 5224 and head 5402. The sensor 5404 comprises multiple sensing elements 322A (Y-axis orientation), 5062 (Z-axis orientation), 5064 (X-axis orientation), which may be independent such as three separate single-axis accelerometers, or these sensing elements may be combined into a single arrangement, such as a three-axis accelerometer. FIG. 60 is diagram 5450 including a side view schematically representing the sensor 5404 of FIG. 59, highlighting the orientation of the sensing elements 322A, 5062.

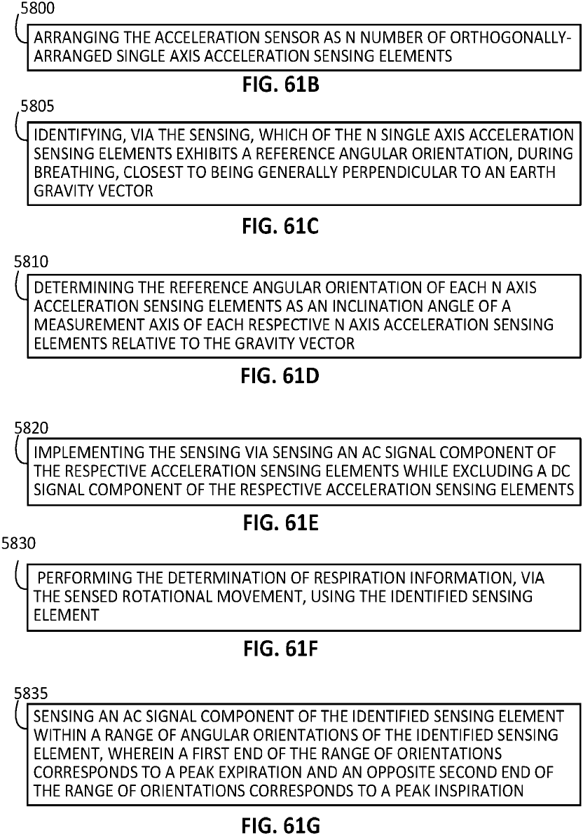

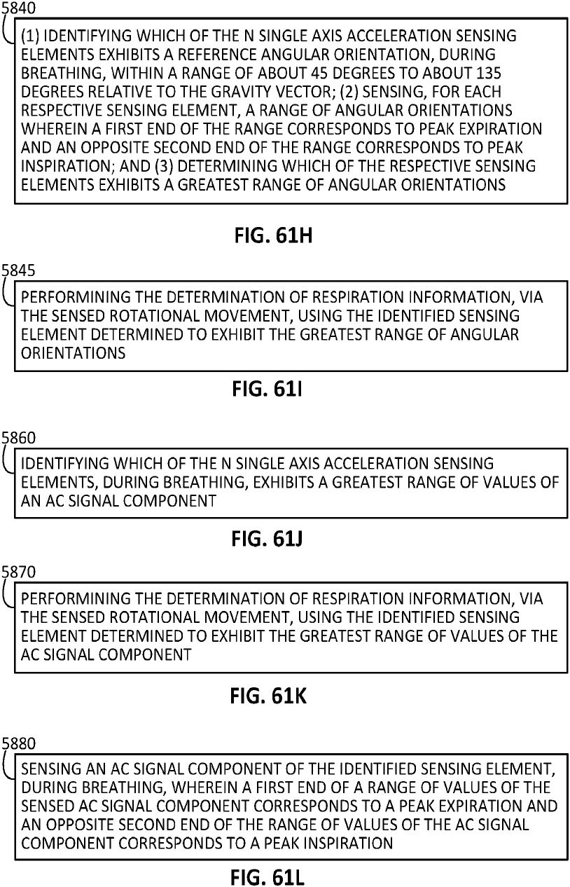

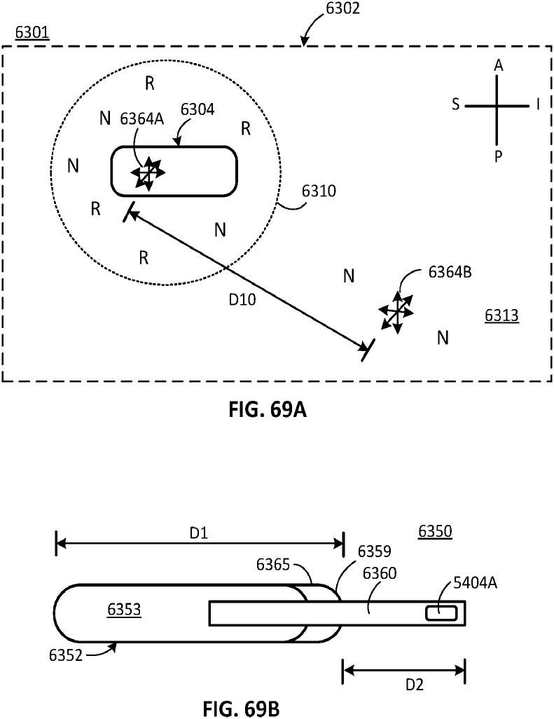

FIG. 61A is diagram 5600 including an isometric view schematically representing an implantable device 5602 comprising an accelerometer-based sensor 5404, which may comprise at least some of substantially the same features and attributes as the sensors, sensing elements, and related example methods as previously described in association with at least FIGS. 3A-3C and FIG. 56A-60. It will be understood that the sensor (and sensing elements) described in FIGS. 3A-3C and FIG. 56A-60 may be implemented as being on or within device 5602. In some examples, sensor 5404 is enclosed within a sealed housing (e.g. can) of the device 5602. However, as described further later in association with at least FIG. 69B, the sensor 5404 may be external to the housing 5605 of device 5602, whether located on the housing or extending from the housing 5605 on a lead.

With further reference to FIG. 61A, in some examples, device 5602 may comprise an implantable device, which includes circuitry and power elements to operate the sensor 5404 to sense physiologic phenomenon, such as but not limited to respiration information. In some examples, the circuitry and power may be implemented within or as part of a control portion 3000 (FIG. 52B) and/or related portions, elements, functions, parameters, engines, as further described later in association with at least FIG. 74-75E. Among other attributes, via the control portion, the device 5602 may be used to monitor and/or diagnose physiologic phenomenon, patient conditions (e.g. respiratory health, cardiac health, etc.), with one such patient condition including sleep disordered breathing (SDB). In some examples, device 5602 may comprise an implantable pulse generator (IPG), which may implement neurostimulation in association with respiration detection in order to treat sleep disordered breathing and/or other patient health conditions. In some such examples, the device 5602 may also sense translational movements of the chest wall and/or associated body tissue in order to sense, monitor, diagnose, etc. the various physiologic phenomenon, patient conditions, etc. whether the sensed translational movement is obtained instead of, or in addition to, the sensed rotational movement of the portion of the chest wall.

In some examples, the sensor 5404 may be mounted or otherwise formed on an external surface (e.g. case) 5605 of the device 5602 (e.g. IPG), or the sensor 5404 may be enclosed within an interior of the device 5602 (e.g. IPG), i.e. within the case.

With the examples of FIGS. 3A-3C and FIGS. 56A-61A in mind, it will be understood that in some examples the sensor signal which will be used to determine respiration information may be selected from among multiple sensing elements, such as but not limited to, the individual axis of the three-axis accelerometer. Accordingly, at least some example methods and/or devices as described in association with at least FIGS. 61B-61L further describe such selection.

Accordingly, as shown at 5800 in FIG. 61B, some example methods and/or devices for determining respiration information may comprise arranging the acceleration sensor as n number of orthogonally-arranged single axis acceleration sensing elements. As shown at 5805 in FIG. 61C, in some examples the method comprises identifying, via the sensing, which of the n single axis acceleration sensing elements exhibits a reference angular orientation, during breathing, closest to being generally perpendicular to the gravity vector. In some such examples, as shown at 5810 in FIG. 61D, the method comprises determining the reference angular orientation of each n axis acceleration sensing elements as an inclination angle of a measurement axis of each respective n axis acceleration sensing elements relative to the gravity vector.

In some examples associated with FIGS. 61A-61D, as shown at 5820 in FIG. 61E, the method comprises implementing the sensing via sensing a AC signal component of the respective acceleration sensing elements while excluding (or at least minimizing) a DC signal component of the respective acceleration sensing elements.

With reference to the example method in at least 5805 in FIG. 61C, the example method may comprise performing the determination of respiration information, via the sensed rotational movement, using the identified sensing element as shown at 5830 in FIG. 61F. In some such examples, the method comprises performing the determination, via the sensed rotational movement, comprises using at least two of the acceleration sensing elements.

In some such examples as previously described in FIGS. 61B-61F, one example method comprises, as shown at 5835 in FIG. 61G, determining the respiration information comprises sensing an AC signal component of the identified sensing element within a range of angular orientations of the identified sensing element, wherein a first end of the range of orientations corresponds to a peak expiration and an opposite second end of the range of orientations corresponds to a peak inspiration. In some examples, the first end of the range of orientations corresponds to the reference angular orientation.

As shown at 5840 in FIG. 61H, in some examples of determining respiration information, the method comprises: (1) identifying which of the n single axis acceleration sensing elements exhibits a reference angular orientation, during breathing, within a range of about 45 degrees to about 135 degrees relative to the gravity vector; (2) sensing, for each respective identified acceleration sensing element, a range of angular orientations relative to the gravity vector, wherein a first end of the range of orientations corresponds to a peak expiration and an opposite second end of the range of orientations corresponds to a peak inspiration; and (3) determining which of the identified acceleration sensing elements exhibits a greatest range of angular orientations. In some examples, method 5840 further comprises, as shown at 5845 in FIG. 61I, performing the determination of respiration information, via the sensed rotational movement, using the identified acceleration sensing element determined to exhibit the greatest range of angular orientations.

In some such examples, such as at 5840, the method may comprise performing the determination, via the sensed rotational movement, comprises using all of the identified acceleration sensing elements. In some examples of at least method 5800 (FIG. 61B) and the associated aspects in FIGS. 61C-61I, the variable n equals 3.

With further reference to the example method shown at 5800 in FIG. 61B, in some examples, as shown at 5860 in FIG. 61J, some example methods comprise identifying which of the n single axis acceleration sensing elements, during breathing, exhibits a greatest range of values for an AC signal component. In some such examples, as shown at 5870 in FIG. 61K, the method comprises performing the determination of respiration information, via the sensed rotational movement, using the identified acceleration sensing element determined to exhibit the greatest range of values of the AC signal component.

With regard to example methods in at least FIGS. 61J and/or 61K, the example method may comprise determining a sensing signal for each n axis acceleration sensing elements as an inclination angle of a measurement axis of each respective n axis acceleration sensing elements relative to the gravity vector, in a manner similar to that previously shown at 5810 in FIG. 61D.

With regard to example methods in at least FIGS. 61J and/or 61K, one example method (as shown at 5880 in FIG. 61L) may further comprise determining the respiration information via sensing an AC signal component of the identified sensing element during breathing, wherein a first end of a range of values of the sensed AC signal component corresponds to a peak expiration and an opposite second end of the range of values of the sensed AC signal component corresponds to a peak inspiration.

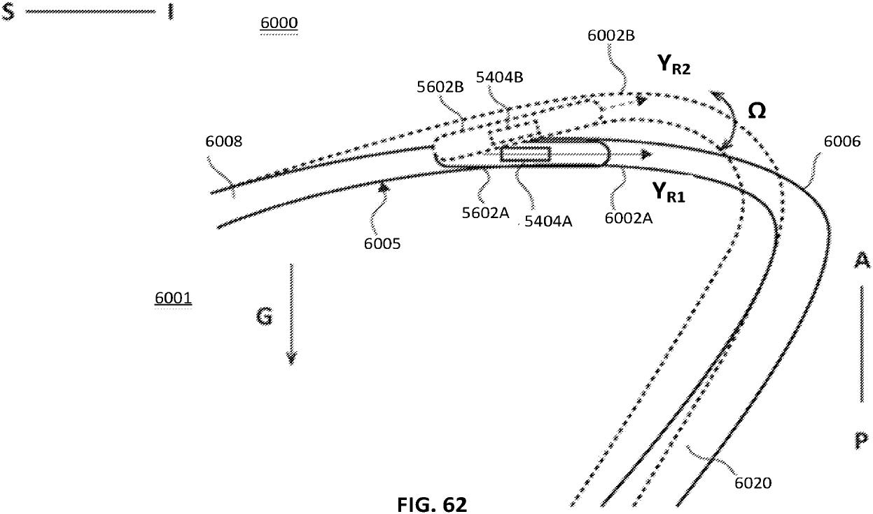

FIG. 62 is a diagram 6000 schematically representing a side view of a patient’s chest in which is implanted an example device 5602A and/or at which example method is performed. As shown in FIG. 62, device 5602A has been chronically, subcutaneously implanted to be coupled relative to a portion 6002A of a patient’s chest wall 6005 of chest 6001. In the particular example shown, the chest wall portion 6002A corresponds to an anterior portion of the rib cage/chest 6001. Meanwhile, the non-bony structures (e.g. fascia, muscle, etc.) overlying the rib cage, and within which the device 5602A may be inserted, are omitted from FIG. 62 for illustrative simplicity and clarity.

In some examples, device 5602A may comprise at least some of substantially the same features and attributes as device 5602 in FIG. 61A, with sensor 5404A comprising at least some of substantially the same features and attributes as the sensing elements described in association with FIGS. 3A-3B and FIGS. 56A-61A.

As shown in FIG. 62, during breathing, the chest wall portion 6002A (shown in solid lines) rises into the position shown in dashed lines 6002B as the rib cage expands upon inspiration and then chest wall portion 6002A falls into the position shown in solid lines as the rib cage contracts during expiration, with the cycle repeating itself with each breath. As further shown in FIG. 62, when the rib cage is in a contracted state (e.g., peak expiration), the sensor 5404 is in a first orientation (as represented by solid line indicator YR1) in a manner similar to that shown in FIGS. 3A-3B. When the rib cage in an expanded state (e.g. peak inspiration), the sensor 5404B is in a second orientation (as represented by dashed line indicator YR2) in a manner similar to that shown in FIGS. 3A-3B. In one aspect, some inferiorly-located portions of chest wall (e.g. 6002A, which expands to position shown at 6002B) exhibit significant movement whereas other more superiorly-located chest wall portions 6008 may remain relatively stationary, such that the chest wall exhibits rotational movement which is sensed by sensor 5404A and which is representative of respiratory behavior of the patient. Accordingly, by employing sensor 5404A to measure an inclination angle (Ω) during such rotational movement of the chest wall portion 6002A during breathing, a suitable respiratory information signal may be obtained.

It will be understood that the device 5602A (and sensor 5404A) is not limited to being implanted strictly at the location of the chest wall (along the superior-inferior orientation) depicted in FIG. 62, but may be closer to the superior end 6008 of the chest wall 6002A provided that a sufficient range of rotational movement of the chest wall (between inspiration and expiration) is detectable via sensor 5404A. Likewise, in some examples, the device 5602 (and sensor 5404A) may be closer to the inferior end 6006 of the chest wall.

Moreover, it will be understood that while device 5602A as shown in solid lines is depicted in a generally horizontal orientation within the FIG. 62, this representation does not limit the implantation of device 5602A to such an orientation. In addition, as previously noted, the effectiveness of the device 5602A (including sensor 5404A) to detect respiration information is not limited to having an exactly horizontal orientation but rather effectuated by the change in angular orientation (e.g. YR1 to YR2, and vice versa) of the inclination angle (Ω in FIG. 3B) of the sensor 5404A, as previously described.

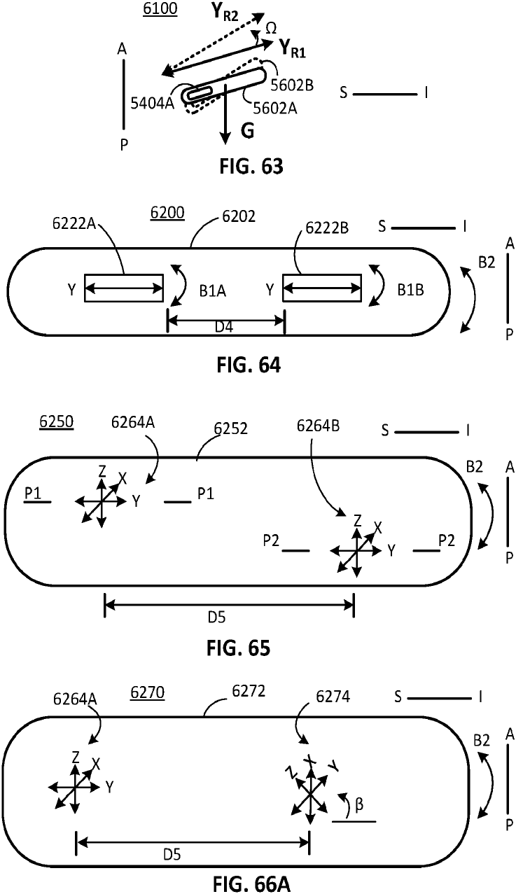

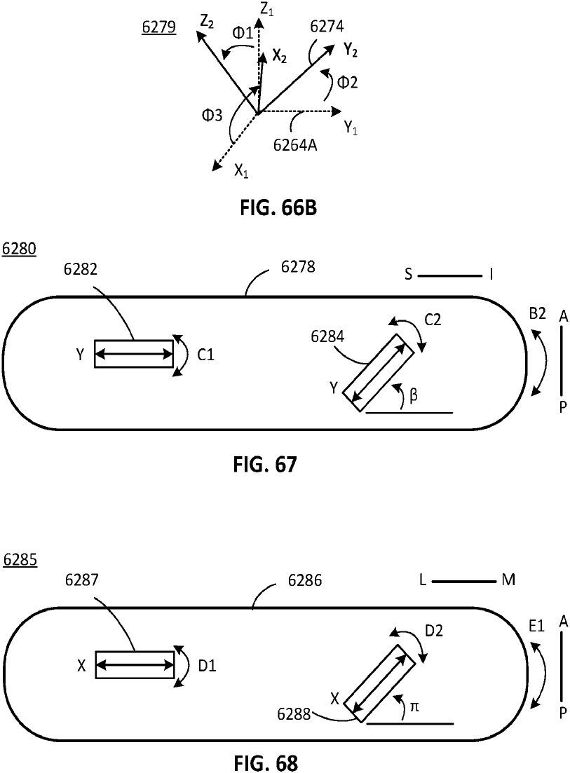

FIG. 63 is a diagram which schematically represents device 5602A (including sensor 5404A) which is deployed in a manner consistent with at least FIGS. 3A-3C and FIG. 56A-62. Among other things, FIG. 63 demonstrates that in at least some instances the device 5602A, and therefore sensor 5404A) may be implanted such that it is has an orientation YR1 which is not generally parallel to a superior-inferior orientation (S-I) of the patient’s chest (and body). Rather, in at least some examples, the orientation YR1 shown in FIG. 63 may result from the natural angle of the portion of chest wall at which the device 5602A (and sensor 5404A) is implanted. Nevertheless, with regard to at least a primary sleep position in which the patient is generally horizontal (e.g. supine, prone, side-laying) the example methods and/or example devices remain effective in detecting respiration information because the primary mechanism of obtaining the respiration information is based on observing the change in value of the AC signal component associated with the measured inclination angle (Ω) through the range of rotational movement between first angular orientation YR1 (e.g. peak expiration) and second angular orientation YR2 (e.g. peak inspiration), in some examples. Accordingly, provided that the first angular orientation YR1 at the time of measuring the signal extends at an appropriate angle relative to the gravity vector G (as extensively described in association with at least FIGS. 3A-3B and FIG. 56A-58) sufficient to obtain an AC signal component which is substantially sensitive to changes in an inclination angle (e.g. Ω in FIG. 3B) of the sensor 5404A (e.g. sensing element 322A in FIG. 3B), then suitable determination of respiration information can be made.

FIG. 64 is a diagram 6200 including a side view schematically representing an example implantable device 6202 and/or example method. In some examples, the example device 6202 (and/or example methods) may comprise at least some of substantially the same features and attributes as the sensors (e.g. 5404A), sensing elements (e.g. 322A), devices (e.g. 5602A) and related example methods as previously described in association with FIGS. 3A-3C and FIG. 56A-63, except further comprising a second sensing element 6222B within device 6202 in addition to a first sensing element 6222A (like 322A in at least FIG. 3B). The two sensing elements 6222A and 6222B are spaced apart by a distance D4 within the device 6202. In some examples, the multiple sensing elements 6222A, 6222B provide multiple sources of respiration information for redundancy and/or to provide more robust sensing.

In some such examples, each sensor 6222A, 6222B may experience slightly different rotational movement and this difference signal may be used to increase sensitivity to angular movement such as occurs during respiration while reducing sensitivity to translational movement such as occurs due to non-respiratory muscle movement, in order to better determine respiration information. For instance, in some example, because the two separate accelerometers (e.g. 6222A, 6222B) are aligned along the same axis (in at least some examples), the two output signals could be subtracted from one another for an estimate of a true gyroscopic or rotational signal of the device (as opposed to the relative projection of the gravity vector). In some instances, two low gain signals (one from each accelerometer) may be added together for greater signal magnitude, may be averaged for reduction of sensor noise, and/or may be subtracted for a common-mode rejection.

FIG. 65 is a diagram 6250 including a side view schematically representing an example implantable device 6252 and/or example method. In some examples, the example device 6252A (and/or example methods) may comprise at least some of substantially the same features and attributes as the sensors (e.g. 5404), sensing elements (e.g. 322A), devices (e.g. 5602) and related example methods as previously described in association with FIGS. 3A-3C and FIG. 56A-63, except further comprising two spaced apart, orthogonally-arranged multiple axes accelerometer sensors 6264A, 6264B which are spaced apart by a distance D5. In some examples, each sensor 6264A, 6264B comprises a three-axis accelerometer with each accelerometer having the same orientation within the device 6252, e.g. the Y-axis sensing element of accelerometer sensor 6264A is generally parallel to the Y-axis sensing element of accelerometer sensor 6264B. In some examples, the respective accelerometer sensors 6264A, 6264B are in same plane (P1), i.e. the Y-axis sensing element of accelerometer sensor 6264A extends in the same plane (P1) as the Y-axis sensing element of accelerometer sensor 6264B. In some examples, this arrangement of providing two spaced apart three-axis accelerometer sensors may provide at least some information approximately the function of a gyroscope, while consuming less power. In addition, by having two separate and independent three-axis accelerometers, the arrangement may provide more robust signal capture.

However, as further shown in FIG. 65, in some examples the respective accelerometer sensors 6264A, 6264B extend in different planes (P1 and P2) within device 6252, i.e. at least one axis sensing element (e.g. Y) of accelerometer sensor 6264A extends in a first plane (P1) which is different than a second plane (P2) in which a corresponding axis sensing element (e.g. Y) of accelerometer sensor 6264B extends. In some examples, this arrangement may enhance signal fidelity. It will be understood that in some examples another axis sensing element (e.g. X) of one accelerometer sensor 6264A also may extend in a plane different from the corresponding axis sensing element (e.g. X) of the second accelerometer sensor 6264B.

FIG. 66A is a diagram 6270 including a side view schematically representing an example implantable device 6272 and/or example method. In some examples, the example device 6272 (and/or example methods) may comprise at least some of substantially the same features and attributes as the sensors (e.g. 5404), sensing elements (e.g. 322A), devices (e.g. 5602) and related example methods as previously described in association with FIGS. 3A-3C and FIG. 56A-65, except further comprising two spaced apart, orthogonally-arranged multiple axes accelerometers 6264A, 6274 which are spaced apart by a distance D5 and which have different orientations within device 6272. In some examples, each sensor 6264A, 6274 comprises a three-axis accelerometer with each accelerometer having different orientations within the device 6272, such as the one axis sensing element (e.g. Y) of accelerometer sensor 6274 not being generally parallel to the corresponding Y- axis sensing element of accelerometer sensor 6264A, but rather the Y-axis sensing element of sensor 6274 extending at an angle (β) relative to the Y-axis sensing element of sensor 6264A. In some examples this angle may comprise about 45 degrees.

In some examples, this angle (β) (by which the orientation of second accelerometer sensor 6274 is offset relative to first accelerometer sensor 6264A) may fall within a range of about - 70 degrees to about 70 degrees and/or within a range in which the sensitivity of the AC signal component of the sensing element(s) (e.g. Y-axis sensing element, etc.) to changes in the inclination angle (e.g. Ω in FIGS. 3B, 62) remains sufficiently accurate and/or of a magnitude to reliably capture respiration information (including respiratory morphology) of the patient during breathing.

In some examples, this example arrangement provides for more robust sensing of respiratory information at least because, regardless of the particular implant angle (e.g. angle of device and sensor relative to the superior-inferior orientation of chest) and/or of the particular patient body position at the time of sensing, at least one of the three sensing axes of the first accelerometer 6264A and at least one of the three sensing axes of the second accelerometer 6274 will extend in an orientation having a sufficiently high sensitivity of an AC signal component of an acceleration signal to enable reliably and accurately measuring a change in inclination angle (Ω in FIG. 3B) of (at least) the at least one sensing axes between a first angular orientation (e.g. YR1 in FIG. 3B) and a second angular orientation (e.g. YR2 in FIG. 3B).

It will be further understood that the second three-axis accelerometer 6274 may be secured within device 5602A at an offset angle (β) relative to the secured position of first three-axis accelerometer 6264A within device 6252 for more than one axis (e.g. Y), such as being offset for two axes (e.g. Y, Z or Y, X) or three axes (e.g. Y, X, and Z), as shown in FIG. 63.

For example, FIG. 66B is a diagram 6279 juxtaposing the respective axes (Z2, Y2, X2) of the sensor 6274 (shown in solid lines) relative to the respective axes (Z1, Y1, X1) of sensor 6264A (shown in dashed lines) to schematically represent a degree (Φ1, Φ2, Φ3) by which each of the respective axes (Z2, Y2, X2) of the sensor 6274 (shown in solid lines) may be offset from the respective axes (Z1, Y1, X1) of sensor 6264A (shown in dashed lines). In some examples, angle Φ1, Φ2, Φ3 all have the same value (e.g. 45 degrees, 50 degrees, 60 degrees, etc.) while in some examples, some of the angles (e.g. Φ1 ) may have a value (e.g. 40 degrees) which is different than a value (e.g. 60 degrees) of another angle (e.g. Φ2).

FIG. 67 is diagram 6280 including a side view schematically representing an example implantable device 6278 and/or example method. In some examples, the example device 6278 (and/or example methods) may comprise at least some of substantially the same features and attributes as the sensors (e.g. 5404A), sensing elements (e.g. 322A), devices (e.g. 5602) and related example methods as previously described in association with FIGS. 3A-3C and FIGS. 56A-66B, except further comprising two spaced apart single axis accelerometer sensing elements 6282, 6284 which are spaced apart by a distance D5 (like in FIGS. 65, 66A) and which have different orientations within device 6278. In some examples, each sensing element 6282, 6284 comprises a single-axis accelerometer with each accelerometer having different orientations within the device 6282, e.g. the acceleration sensing element 6282 (e.g. Y) being not generally parallel to the acceleration sensing element 6284 (e.g. Y) but rather the sensing element 6284 extending at an angle (β) relative to the sensing element of sensor 6282. Other than comprising two single axis acceleration sensors instead of two three-axis acceleration sensors (FIG. 66A), the device 6282 may comprise at least some of substantially the same feature and attributes as device 6272 in FIGS. 66A-66B.

In a manner similar to the example of device 6278 in FIG. 67, in some examples, an implantable device 6286 as shown in FIG. 68 may comprise the same type of example arrangement to provide two single-axis acceleration sensing elements (6287, 6288) where the offset angle (π) is implemented relative to an x-axis extending in the lateral-medial orientation (L-M) of the patient’s chest.

FIG. 69A is a diagram including a top plan view schematically representing an example method 6301 (and/or device) including two separate acceleration sensors 6364A, 6364B, which are implanted within a patient’s body 6302. In some examples, both the first and second acceleration sensors 6364A, 6364B comprise at least some of substantially the same features as the acceleration sensor described in association with at least FIGS. 1A-3B, 56A-68, etc. In some examples, the respective sensors 6364A, 6364B are spaced apart by a distance D10 such that a first acceleration sensor 6364A is positioned within a region 6310 of the patient’s body 6302 in which the first acceleration sensor 6364A readily senses respiration (R) of the patient while the second acceleration sensor 6364B is implanted within the patient’s body 6307 in a region 6313 which does not readily sense the patient’s respiratory behavior (R). However, the distance D10 corresponds to a distance at which both the respective first and second acceleration sensors 6364A, 6364B are positioned in the patients’ body 6302 in a manner in which they both may experience substantially the same noise (N) which is substantially the same.

In view of this arrangement, the second signal sensed via the second acceleration sensor 6364B (which senses noise without respiratory information) can be subtracted from the first signal sensed via first acceleration sensor 6364B (which senses both respiration and noise) to produce an effective signal which represents sensed respiratory information without the noise N common to both regions 6310, 6313 of the patient’s body.

In some such examples, the sensing arrangement described in association with FIG. 69A may comprise one example implementation of subtracting or other neutralizing noise according to the noise model 7470 in FIG. 75D, the noise model 7596 in FIG. 75E, example method 7885 in FIG. 85, and/or example method 7890 in FIG. 86. In this way, more accurate and effective detection of respiratory information may be obtained, which in turn may produce more accurate, effective identification of disease burden indicators, such as but not limited to sleep disordered breathing.

FIG. 69B is a diagram 6350 including a top view schematically representing an example implantable device 6352. In some examples, device 6352 may comprise at least some of substantially the same sensing elements (e.g. 322A), devices and related example methods as previously described in association with FIGS. 3A-3C and FIG. 56A-68, except comprising placement of the acceleration-based sensor 5404A externally to the housing 6355 of the implantable device 6352. In some such examples, the sensor 5404A may comprise a portion of a lead 6360 which is coupled (e.g. electrically and mechanically) relative to the implantable device 6352 via a feedthrough portion 6353. In some examples, the lead 6360 (including sensor 5404A) may extend a distance D2 from edge 6359 of housing 6355, which is about the same as or less than a greatest dimension (e.g. D1) of the housing 6355. Via this example arrangement, the sensor 5404A (e.g. accelerometer and/or other) may be located externally from the housing 6305 of device 6302 yet still be close enough to the housing 6355 such that both the lead 6360 (including sensor 5404A) and the device 6352 may be implanted within a single (e.g. same) subcutaneous pocket, such as (but not limited to) a pocket within the pectoral region.

However, in some examples, the lead 6360 may be longer than distance D2 and be placed subcutaneously via tunneling such that the lead 6360 (and sensor 5404A) extends beyond a subcutaneous pocket in which the device 6352 is implanted.

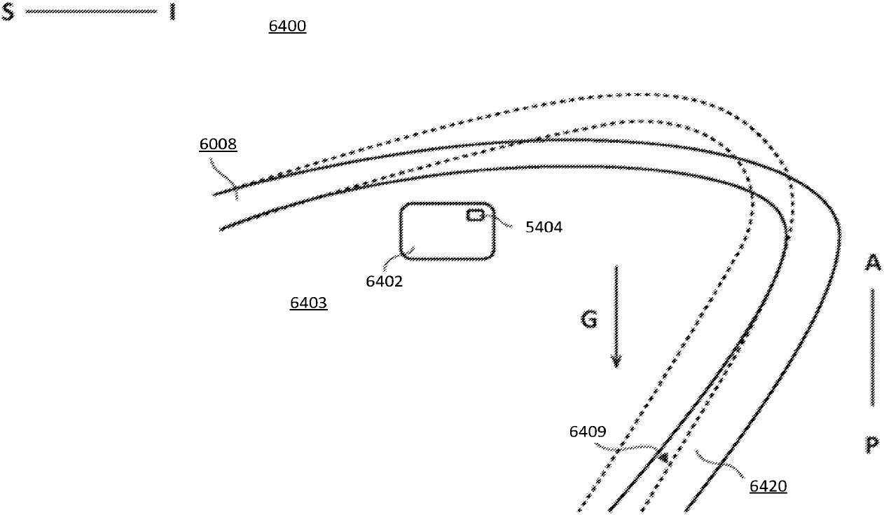

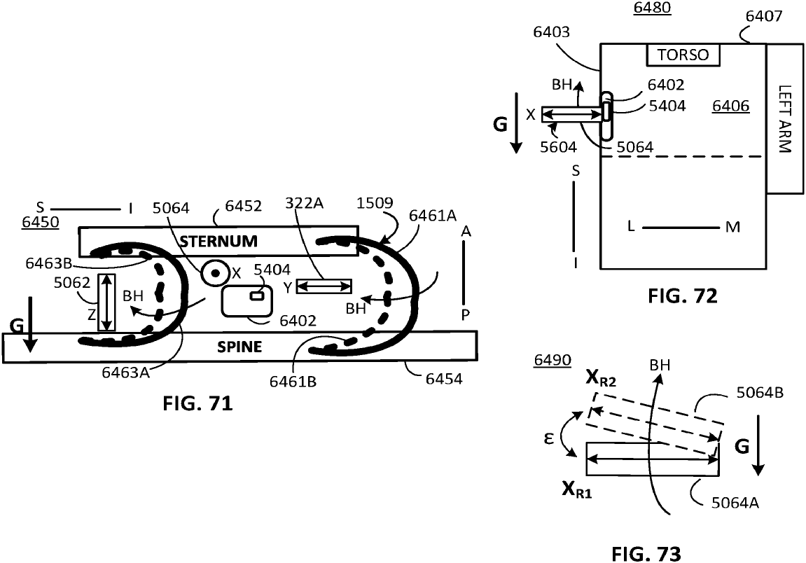

As described in association with FIGS. 70-73, in some examples an implantable device 6402 may be implanted on a side portion of the patient’s rib cage and used in an example method to detect respiration information. In some examples, the example device 6402 and/or example method may comprise at least some of substantially the same features and attributes as the sensors (e.g. 5404A), sensing elements (e.g. 322A), devices (e.g. 5602) and related example methods as previously described in association with FIGS. 3A-3C and 56A-69, except for at least device 6402 being implanted on the side portion 6403 of the patient’s rib cage 6409 as shown in FIG. 70. Among other attributes, placement of device 6402 on a side portion 6403 of the patient’s rib cage 6409 may enhance sensing a bucket-handle-type rotational movement of the rib cage during breathing (as represented via directional arrow BH in FIGS. 71-73), which may result in a significant change in an inclination angle measurable via an acceleration sensing element 5064 having a primary orientation in lateral-medial orientation (L-M) of the rib cage. In some examples, such example arrangements may be particularly useful in an example method such as FIGS. 57A-57C and 58 in which an x-axis orientation acceleration sensing element has a measurement axis aligned generally parallel to a lateral-medial orientation (L-M) of the rib cage, and the patient’s body is in a fully vertically upright position (FIG. 58) or a partially vertically upright position (FIG. 57A).

FIG. 71 is a diagram including a side view schematically representing an example device 6402 (FIGS. 70-71) mounted on a side portion 6403 (e.g. lateral portion) of a patient’s rib cage 6409. As shown in FIG. 71, during breathing the example ribs 6461A, 6463A (shown in solid lines) rotationally move from a first position (e.g. peak expiration) to a second position 6461B, 6463B (shown in dashed lines) corresponding to peak inspiration, as represented via directional arrow BH. As further shown in FIG. 71, the example ribs 6461A, 6463A extend in a curved manner between a sternum 6452 at a front of the rib cage 6409 (e.g. chest) and a spine 6454 at a back or rear of the rib cage 6409. As previously noted, in some sleeping positions the particular implant location may position at least some of the acceleration sensing elements (e.g. along a lateral-medial (L-M) orientation) to enhance sensing respiration information, including respiration morphology.

It will be understood that the ribs will return from their position (dashed lines 6461B, 6463B) at peak inspiration to the position shown in solid lines 6461A, 6463A corresponding to peak expiration, as the patient’s respiratory cycle repeats cycles of inspiration followed by expiration.

FIG. 72 is a diagram 6480 including a front view schematically representing the example device 6402 of FIGS. 70-71 implanted along a side portion 6403 of the patient’s ribcage 6409 and illustrating an orientation of rotational movement, according to a bucket-handle-type of motion (arrow BH) along the side portion 6403 of the rib cage 6409 during breathing. In some examples, as previously mentioned and as shown in FIG. 72, at least an X-axis acceleration sensing element 5064 of sensor 5404 of device 6402 may extend generally perpendicular to such bucket-handle-type (BH) of rotational movement of the side portion 6403 of rib-cage 6409.

As further shown in FIG. 72, in situations in which the patient may be sleeping in a partially upright position (FIG. 57A) or a fully upright position (FIG. 58) and sleeping, the x-axis acceleration sensing element 5064 (of a side-mounted device 6402 of FIGS. 70-72) may be the sensing element (of a three-axis accelerometer or of multiple accelerometers) which exhibits the highest sensitivity for an AC signal component in measuring an inclination angle of sensing elements during breathing. In some such example arrangements, as shown in the diagram 6490 of FIG. 73, the x-axis acceleration sensing element 5064 may move between a first orientation XR1 (shown as 5064A in solid lines) corresponding to peak expiration and a second orientation XR2 (shown as 5064B in dashed lines) corresponding to peak inspiration in order to sense an inclination angle (ε) through a range of motion during breathing, with the sensed signal being proportional to and representative of respiration morphology of the patient.

With further reference to FIGS. 70-73, in some examples, an implant location of an example device (e.g. 5602 in FIG. 62, 6402 in FIGS. 70-72) may comprise a hybrid location on a front/top of chest and on a side of chest, such as front “corner” of the rib cage. In some such example arrangements, this “corner” implant location may capture some of both a bucket-handle-type rotational movement (side of rib cage) as in FIGS. 70-73 and a rise-fall-type rotational movement on front of chest as in FIGS. 3A-3B and FIG. 56A-69.

It will be understood that in some example implementations, such rotational movement sensing (to determine respiration information) may be performed via sensing element(s) at both a front or top portion of a chest (e.g. FIG. 62) and a lateral portion of a chest (e.g. FIGS. 70-73), In some such examples, the rotational sensing information from both sensing locations may be combined to provide more robust and/or accurate respiration determination. However, some example methods and/or devices may use sensed rotational movement (caused by breathing) from just one of the sensing locations based on which sensing location produces the most robust and/or useful respiration information at a given point in time, and the particular sensing location (e.g. top/front portion or lateral portion) being used (to determine respiration information) at any particular point in time may vary.

FIG. 74 is a block diagram schematically representing example method 7300. In some examples, method 7300 may be implemented via at least some of the devices, sensors, sensing elements, etc. as described in association with FIGS. 1A-74 and 75B-102. In some examples, the example method 7300 may be at least partially implemented within, and/or via, control portion 3000 in FIG. 52B, control portion 3020 in FIG. 52C, user interface 3040 in FIG. 52D, 3050 in FIG. 52E, care engine 2900 in FIG. 52A. In some such examples, the example method may be implemented as part of (and/or via) sensing portion 2910 and/or respiration portion 2912 of care engine 2900 in FIG. 52A.

As shown at 7310 in FIG. 74, the example method 7300 comprises sensing acceleration signal(s) from a sensor(s) implanted within a patient’s body in a position, such as in the chest region, to detect respiration information. In some examples, just a single sensing element (e.g. 322A in FIG. 3B) may be used to provide just a single sensed acceleration signal or in some examples, multiple sensing elements may be used to provide separate multiple sensed acceleration signals. The multiple sensing elements may be separate from, and independent of, each other, or may be co-located as part of a single device, such as a three-axis accelerometer.

As further shown at 7314, filtering is applied separately to the sensed signal(s) (7310) to produce a respective separate inclination angle signal (7321X, 7321Y, 7321Z) for each corresponding acceleration signal (e.g. X-axis, Y-axis, Z-axis). It will be understood that if just one single-axis sensing element is employed, then just one inclination angle signal will be present at 7320. As previously described through various examples, the inclination angle signal represents the physiologic phenomenon of the patient’s breathing with a value and/or shape of the inclination angle signal varying through the different phases of a respiratory cycle (e.g. inspiratory phase, expiratory active phase, expiratory pause phase) as the patient breathes. It will be further noted that while some examples may comprise tracking inclination angle signal for multiple axes (X, Y, Z), some example methods may focus on an axis which is closest to being generally perpendicular to the gravity vector.

In some examples, in addition to applying filtering (at 7314) as described above to produce a respective separate inclination angle signal (7321X, 7321Y, 7321Z) for each corresponding acceleration signal (e.g. X-axis, Y-axis, Z-axis), the filtering may further comprise subtracting (e.g. filtering, excluding) noise from the signal to increase the signal-to-noise ratio for the respiratory features of interest. In some examples, such noise filtering may be implemented as described later in association with noise model 7470 in FIG. 75D. It will be understood that in some examples, such noise filtering may be applied in other ways and/or at other times within the example method (and/or arrangement) in FIG. 74.

As further shown at 7340 in FIG. 74, method 7300 comprises performing a feature extraction a signal-by-signal basis (7341X, 7341Y, 7341Z) to identify within each inclination angle signal (7321X, 7321Y, 7321Z) features indicative of respiration (and/or other features pertinent to respiratory detection, patient health, etc.). As shown at 7350, in some examples the method identifies at least respiratory phase information including (but not limited to) the features of an inspiratory phase 7352, an expiratory active phase 7354, and an expiratory pause phase 7356. It will be understood that each feature (e.g. phase 7352, 7354, 7356) may comprise a start (i.e. onset), an end (e.g. offset), duration, magnitude, and/or both a “start and end” of each respective feature. In some instance, a particular feature may be sometimes be referred to as a fiducial or similar terms, such as a start of a phase (e.g. inspiration) comprising a fiducial.

As shown at 7330, a confidence factor may be applied to each of the feature extraction elements (7341X, 7341Y, 7341Z), such as an X-axis confidence factor 7331X, Y-axis confidence factor 7331Y, and Z-axis confidence factor 7331Z. At least some aspects of applying a confidence factor are described later in association with at least FIG. 75A.

In some examples, upon performing feature extraction (7341X, 7341Y, 7341Z) of respiratory phase information to each inclination angle signal, the resulting extracted feature signals are combined (e.g. fused together) at 7345 to produce (i.e. determine) a composite sensed respiratory signal including respiratory phase information (7350) including inspiratory phase 7352, expiratory active phase 7354, and expiratory pause phase 7354. In some examples, the different extracted feature signals may be combined (e.g. fused) as an average of the respective features, a median of the respective features, or weighting (linear or non-linear) according to a confidence factor (e.g. 7331X, 7331Y, 7331Z). At least some aspects of the confidence factor(s) are described later in association with at least FIG. 75A. In some such examples, the composite sensed respiratory signal may correspond to the virtual vector as previously described in association with at least FIG. 56A, composite parameter 7533 in FIG. 75E, and throughout various examples of the present disclosure.

As further shown in FIG. 74, from the determined respiratory phases (7352, 7354, 7354), additional respiratory parameters 7360 may be determined. For example, an (overall) expiratory phase may comprise a sum or combination of the expiratory active phase (7354) and the expiratory pause phase (7356). In addition, a respiratory period may be determined from a sum of duration of the inspiratory phase 7352 and a duration of the (overall) expiratory phase, including both the active and pause phases 7354, 7356. Meanwhile, the respiratory rate (RR) may computed as 1/respiratory period. Additional parameters may comprise a computed I/E ratio, such as inspiratory phase duration (Ti in FIG. 3C) divided by an expiratory phase duration (T_{EA} plus T_{EP} in FIG. 3C).

In some examples, assuming a given body position or posture and excluding translational motion along the axes (e.g. X, Y, Z), some additional parameters may be determined from the extracted features (including respiratory phase information at 7350) with such additional parameters comprising: an approximation of a tidal volume as being proportional to acceleration; an approximation of respiratory flow as being proportional to a derivative of the acceleration signal with respect to time; and/or an approximation of minute ventilation as being proportional to a result of a multiplication of the computed volume and the computed respiratory rate (described above).

In some examples, determinations relating to feature extraction (7340 in FIG. 74) may further comprise the following parameters. For instance, in some examples of feature extraction, a signal midpoint may be determined, which comprises an average of previous “n” positive peak values and previous “n” negative peak values, where “n” is 1 or more. In some examples of feature extraction, a signal midpoint crossing may be determined, which comprises a sample at which the signal midpoint is crossed. In some examples, the signal midpoint crossing may involve hysteresis with a hysteresis threshold being determined by a fixed threshold, a fraction of recent “n” peak-to-peak values, a fraction of signal root-mean-square (RMS) value, and/or a dynamic threshold with linear decay or exponential decay. In some examples of feature extraction, a peak midpoint area may be determined which comprises an integral (e.g. sum) of all points from a previous signal midpoint crossing to a current signal midpoint crossing.

In some examples, determination of the expiratory active phase (7354 in FIG. 74) is at least partially based on: (1) a detected peak following Peak-Midpoint Area above mean of “n” recent Peak-Midpoint Areas, wherein the expiratory pause phase 2356 creates a relatively larger Peak-Midpoint area, which allows determination of respiratory phase in a way that is insensitive to signal inversion; (2) an absolute value of a derivative (current sample minus previous sample) above a threshold; and/or (3) an absolute value of a derivative of the signal above a threshold for a time threshold.

In some examples, determination of the expiratory pause phase (7354 in FIG. 74) is at least partially based on: (1) a previous phase detected as an expiratory active phase 7354; (2) an absolute value of derivative of the signal below a threshold; and/or (3) an absolute value of derivative of the signal below a threshold for a time threshold.

In some examples, determination of the inspiratory phase (7352 in FIG. 74) is at least partially based on: (1) a previous phase detected as expiratory pause phase; (2) an absolute value of derivative of the signal above a threshold; and/or (3) an absolute value of derivative of the signal above a threshold for a time threshold.

With further reference to FIG. 74, in some examples determining respiration information (via sensing acceleration signals to detect rotational movements of the ribcage during breathing), the example method 7300 may utilize default respiratory phase values as shown at 7390 instead of using the sensed acceleration signals 7310. For instance, in cases in which the sensed acceleration signal quality is poor (i.e. inadequate), the current respiratory phases of the patient may not be known from the current sensed acceleration signals or recent sensed acceleration signals. In some examples, the default respiratory phase values (7390) are assigned a confidence level or factor 7391, which may have a low value to ensure that extracted features (7341X, 7341Y, 7341Z) are used when the sensed acceleration signal quality is adequate. Accordingly, when the sensed acceleration signal is of sufficient quality as determined by the signal-to-noise ratio of the signal, then method 7300 may ignore the default respiratory phase values at 7390. The signal-to-noise ratio may be determined by a comparison with a typical signal morphology, a comparison with a typical signal frequency content, or by other means.

With further reference to the default respiratory phase values portion 7390 in FIG. 74, in some examples the default respiratory phase values (7390) may be determined using at least one of the following: (1) mean respiratory phase time values of the overall human population; (2) the patient’s historical or recent mean/median respiratory phase and/or phase time values; and (3) intentionally applying a longer respiratory rate or a shorter respiratory rate to decrease the chance that an appreciable number of consecutive stimulation “off” times may align with inspiration.

Accordingly, via the default respiratory phase values, some example methods may comprise substituting, upon the sensor obtaining an inadequate signal, stored respiratory information comprising historical respiration information for at least one of: the patient’s respiratory cycle information; and multiple-patient respiratory cycle information. In some such examples, the patient’s respiratory cycle information comprises a respiratory period, and an example method comprises: creating a modified respiratory period by adding a random time value to the respiratory period of the patient’s respiratory cycle information; and implementing the substituting of the stored respiratory information using the modified respiratory period. In some examples, the random time value may comprise about 0 to about 1 second. In some examples, the random time value may comprise other time periods. In some examples, adding the random time value may cause a result similar that noted above (in regard to the default respiratory phase values) by which the example method may intentionally apply a longer respiratory rate or a shorter respiratory rate to decrease the chance that an appreciable number of consecutive stimulation “off” times may align with inspiration.

In some examples, the method may comprise substituting, upon the sensor obtaining an inadequate signal, stored respiratory information comprising respiratory cycle information including at least one of: a first respiratory rate substantially faster than the patient’s average respiratory rate; and a second respiratory rate substantially slower than the patient’s average respiratory rate. In some such examples, the terms substantially faster and/or substantially slower may correspond to a difference on the order of 5 percent difference, 10 percent difference, and the like.

FIG. 75A is a block diagram schematically representing an example confidence factor portion 7400, which may be employed at 7330 in example method 7300 and/or as part of (or via) control portion 3000 in FIG. 52B. It will be understood that all or just some of the factors (e.g. different combinations or a single factor) in confidence factor portion 7400 may be applied at 7330 in method 7300 in FIG. 74. In some examples, a confidence factor may be implemented as an estimated probability of correctness.

As shown in FIG. 75A, in some examples confidence factor portion 7400 comprises a first factor portion 7410 comprising a signal-to-noise ratio parameter 7412, a threshold parameter 7414, and a recent history parameter 7416. Accordingly, in some examples, via signal-to-noise ratio information (parameter 7412), a confidence level may be determined for each extracted feature (at 7340 in FIG. 74) and/or for each inclination angle signal (at 7320 in FIG. 74). In some examples, at 7414 method 7300 comprises the confidence comprising an amount by which a value (e.g. of a feature, of the inclination signal, etc.) exceeds a threshold. Stated differently, if a value of the inclination signal such as for a particular axis (e.g. Y-axis in FIG. 3B) exceeds a threshold by a significant amount, then the method can apply a high value confidence factor to the Y-axis feature extraction (7341Y in FIG. 74) such that determination of the respiratory phase information (7350 in FIG. 74) may depend primarily on the Y-axis inclination signal (7321Y in FIG. 74) as compared to other axes (e.g. X or Z) inclination signals, if present. In some examples, the confidence factor may be applied per recent history parameter 7416 according to a difference between a current value of an extracted feature and a mean value of “n” recent extracted features.

In some examples, each of the confidence parameters in first factor portion 7410 may be applied quantitatively according to a look-up table, multiplication factor (e.g. 1.5, 2x, etc.), and the like.

In some examples, confidence factor portion 7400 may comprise a second factor portion 7420 by which confidence in a value of a particular extracted feature (7341X, 7341Y, 7341Z) may be increased or decreased based on posture (7422) at the time of sensing, heart rate (7424), and/or sleep stage (7426). As further shown in third factor portion 7430 of FIG. 75A, such confidence factors in second factor portion 7420 may be weighted and/or calibrated according to particular patient-based factors, such as patient preferences (e.g. feedback) 7432, clinician input 7434, and/or other information such sleep study information. Further parameters which may comprise part of second confidence factor portion 7420 may include sensed body temperature, time of day, etc.

In some examples, the various parameters, etc. of the respective first, second, and third portions of confidence factor portion 7400 may be used together in different combinations and/or organized in different groupings (or no groupings) than shown in FIG. 75A.

FIG. 75B is a block diagram schematically representing an example feature extraction portion 7450, which may comprise functions, settings, etc. which may act as part of the implementation of the feature extraction at 7340 in method 7300 of FIG. 74. As shown via parameter 7452 at 7450 in FIG. 75B, in some examples a threshold factor may be applied by a user or clinician to adjust thresholds used in performing feature extraction of the inspiratory phase 7352 (e.g. inhalation threshold), of the expiratory active phase 7354 (e.g. exhalation threshold), and/or of the expiratory pause phase 7356 (e.g. exhalation threshold). As shown via parameter 7454 at 7450 in FIG. 75B, in some examples a sensitivity factor may be applied by a user or clinician to adjust thresholds used in performing feature extraction of the inspiratory phase 7352 (e.g. inhalation sensitivity), of the expiratory active phase 7354 (e.g. exhalation sensitivity), and/or of the expiratory pause phase 7356 (e.g. exhalation sensitivity). In some examples, the sensitivity factor may comprise an invert function to adjust thresholds using in a peak-midpoint calculation of the expiratory active phase 7354.

In some examples, in determining the respiratory phase information (7390) example method 7300 also may comprise predicting an inspiratory phase (e.g. 7352 in FIG. 74), as shown at 7460 in FIG. 75C. The prediction of the inspiratory phase may be used to increase a likelihood of implementing actions (e.g. start of stimulation, etc.) which are to be synchronized with a start of the inspiratory phase 7352. Stated differently, predicting the inspiratory phase 7352 as at 7460 in FIG. 75C may decrease a chance that detection of a start of the inspiratory phase might be missed. Moreover, in some example methods and/or example devices, electrical stimulation of a nerve (e.g. hypoglossal nerve) may be initiated prior to a start of inspiration to ensure that the upper airway is open prior to the pressure applied on the upper airway once the actual inspiratory phase commences. In addition, starting electrical stimulation prior to the actual inspiratory phase also may provide some assurance in cases in which prediction of the inspiratory phase may be incorrect or may experience an insufficient signal-to-noise ratio. In some such examples, example methods and/or devices may initiate the stimulation a predetermined period of time prior to an onset of the inspiratory phase. In some examples, the predetermined period of time has a duration less than a duration of the expiratory pause according to an average duration of an expiratory pause phase, according to a duration of the preceding expiratory pause phase, etc. In some examples, the predetermined period of time may comprise an absolute amount of time (e.g. start 0.5 seconds) and in some examples, the predetermined period of time may comprise a relative amount of time, such as 10% of the preceding respiratory period. As mentioned in association with other examples regarding synchronization, in some examples the predetermined period of time may be about 200 milliseconds, or 300 milliseconds.

In some examples, the inspiratory phase prediction function (7460) in FIG. 75C may comprise predicting a start of the inspiratory phase via timing based on: (1) an expiratory active phase 7354 of the most recent (e.g. immediately preceding) respiratory cycle; (2) an expiratory pause phase 7356 of the most recent (e.g. immediately preceding) respiratory cycle; and/or (3) an inspiratory phase of one or more previous respiratory cycles and/or the respiratory rate of one or more previous respiratory cycles. In some examples, in determining the timing (of the inspiratory phase and/or respiratory rate of previous respiratory cycles), the method may utilize a mean value, a median value, linear extrapolation, and/or non-linear extrapolation of the respective inspiratory phase or respiratory rate.

With further reference to inspiratory phase prediction 7460 in FIG. 75C, in some examples, determining the timing (of the inspiratory phase and/or respiratory rate of previous respiratory cycles), the use of values from previous respiratory cycles may also enhance an accuracy of feature extraction (7340 in FIG. 74). For instance, accuracy of timing peak detection may be enhanced by using data before and after the peak. In another instance, using values from previous respiratory cycles may make an example method (of detecting respiration) less susceptible to a noisy signal during a particular respiratory cycle, patient limb movements, bed partner movements, etc.

In some examples, a method may increase accuracy of determining respiration from a sensed acceleration signal (of rotational movement at a portion of a chest wall) by removing noise from the sensed signal according to a noise model, which is shown in association with at least noise model 7470 in FIG. 75D.

In some such examples, the method comprises constructing the noise model from identifying characteristics (e.g. signal morphology, frequency content, etc.) within the sensed signal which are caused by and/or associated with conditions, phenomenon, etc. other than respiration-related behavior of the patient (and/or cardiac-related behavior, etc.) and which are considered noise relative to the signal of interest regarding patient respiration. In just one example, one source of noise (which may form at least part of a noise model) may comprise movement, behavior, etc. from another person (i.e. partner) sleeping in the same bed, which may be picked up by the sensed signal for the patient. In some instances, such motion may sometimes be referred to as non-patient-physiologic motion. Other sources of noise, which form at least part of a noise model, may comprise additional/other non-patient-physiologic motion, such as but not limited to motion of a vehicle in which the patient is present such as when the patient is traveling a car, airplane, spaceship, etc. Other types of non-patient-physiologic motion which may be considered as noise (and which form at least part of a noise model) may comprise movement of a patient support surface, such as a hammock, swings, etc. Another type of noise, which may form at least part of the noise model, may comprise a physical position of the patient such as being in a very tall building in motion due to wind, a location experiencing vibration or movement such that the motion of the patient may affect the sensed acceleration signal and otherwise hinder accurate determination of respiration information per the type of rotational sensing in the examples of the present disclosure.

By constructing a noise model from these non-patient characteristics, and subtracting the noise model from the sensed acceleration signal of the patient, a more accurate sensed respiration signal may be determined. In some instances, the subtraction may be performed by filtering the noise and/or by excluding sensor element signals including such noise.

In some examples, such noise may be filtered or excluded from the sensed acceleration signals (of rotational movement of a respiratory body portion, such as a chest wall) without use of a formal noise model.

In some examples, at least some of the features and attributes of use of a noise model, which may increase a signal-to-noise ratio of the signal of interest (respiration information), may be implemented at least partially within or via filtering 7314 in method 7300 as shown in FIG. 74.

In some examples, the prediction of the inspiratory phase (e.g. 7460 in FIG. 75C) also may be performed according to cross-referencing (e.g. similarity) the inspiratory phase of a previous respiratory cycle relative to stored reference morphology of the inspiratory phase.

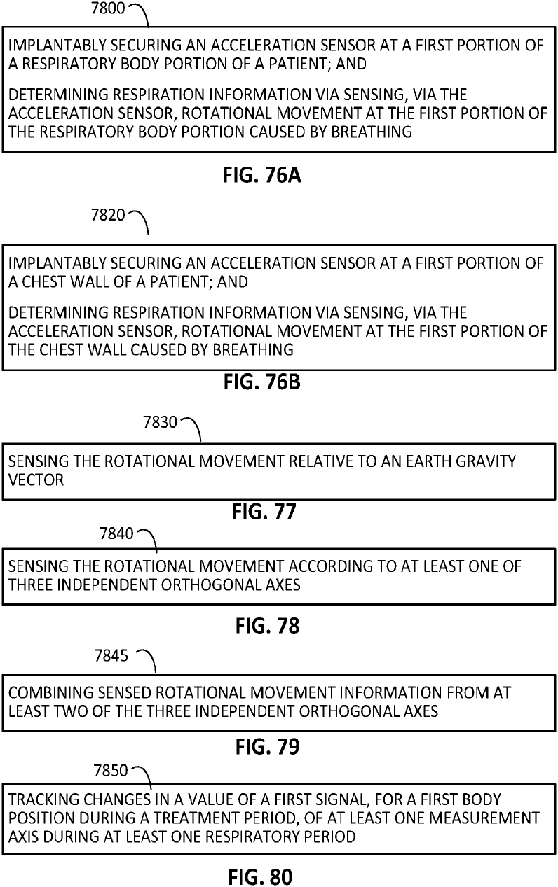

FIG. 75E is a block diagram schematically representing an example care engine 7500. In some examples, the care engine 7500 may form part of a control portion 3000, as previously described in association with at least FIG. 52B, such as but not limited to comprising at least part of the instructions 3011 and/or information 3012. In some examples, the care engine 7500 may be used to implement at least some of the various example devices and/or example methods of the present disclosure as previously described in association with FIG. 1-75D and/or as later described in association with FIG. 76A-102. In some examples, the care engine 7500 (FIG. 75E) and/or control portion 3000 (FIG. 52B) may form part of, and/or be in communication with, a pulse generator (e.g. 2833 in FIGS. 50-51).

As shown in FIG. 75E, in some examples the care engine 7500 comprises a sensing portion 7510, a respiration portion 7580, a sleep disordered breathing (SDB) parameters portion 7600, and/or a stimulation portion 7700. In some examples, the care engine 7500 may comprise one example implementation of care engine 2900 in FIG. 52A. In some such examples, the sensing portion 7510 (FIG. 75E) may comprise one example implementation of the sensing engine 2910 (FIG. 52A), the respiration portion 7580 (FIG. 75E) may comprise one example implementation of the respiration engine 2912 (FIG. 52A), the sleep disordered breathing (SDB) parameters portion 7600 (FIG. 75E) may comprise one example implementation of the SDB parameters engine 2916 (FIG. 52A), and/or the stimulation portion 7700 (FIG. 75E) may comprise one example implementation of the stimulation engine 2918 (FIG. 52A).

In one aspect, at least the sensing portion 7510 of care engine 7500 in FIG. 75E directs the sensing of information, and/or receives, tracks, and/or evaluates sensed information obtained via one or more of the sensors, sensing elements, sensing modalities, etc. as described in association with at least FIGS. 1A-75D and FIG. 76A-102, with care engine 7500 employing such information to determine respiration information, among other actions, functions, etc. as further described below.

In some examples, the sensing portion 7510 may comprise an ECG parameter 7520 to direct ECG sensing, obtain sensed ECG information, etc. to obtain cardiac information and/or some respiration information, which may be used together with respiration information determined via sensing according to the examples in FIGS. 1A-75D and FIG. 76A-102. In some examples, the ECG information is sensed via at least some of the sensing electrodes (e.g. 2812, 2820, 2830, etc.) as previously described in association with at least FIGS. 50-51.

In some examples, the sensing portion 7510 may comprise an accelerometer portion 7530. In some examples, the accelerometer portion 7530 directs acceleration-based sensing, obtains/receives acceleration signal information, etc. to obtain at least respiration information and/or other information (cardiac, posture, etc.). In some examples, such acceleration sensing may be implemented according to at least some of substantially the same features and attributes as described in Dieken et al., ACCELEROMETER-BASED SENSING FOR SLEEP DISORDERED BREATHING (SDB) CARE, published as U.S. 2019-0160282 on May 30, 2019, and which is incorporated by reference herein in its entirety.

In some examples, the acceleration sensing may be used to determine and/or receive inclination information (parameter 7532 in FIG. 75E), such as the changes in inclination angle of the acceleration sensing elements, which is indicative of rotational movement of the patient’s chest wall, which in turn provides respiration information, as extensively described throughout examples of the present disclosure. As noted elsewhere, sensing of rotational movement (to determine respiration information) is not limited solely to the chest (e.g. chest wall) but may comprise other or additional respiratory body portions, such as but not limited to the abdomen (e.g. abdominal wall).

As represented via composite parameter 7533 in FIG. 75E, in some examples, the rotational movement information from at least two of three acceleration sensing elements (e.g. 322A/Y, 5062/Z, 5064/X) may be combined to produce composite rotational movement information (5252), such as previously described in association with at least FIG. 56C. In some instances, the rotational movement information from the combined acceleration sensing signals may sometimes be referred to as a virtual vector, e.g. a virtual rotational movement vector. Via such examples, at least two of the three orthogonally-arranged sensing elements may be used to perform determination of the respiration information at least based on an AC component of a multi-dimensional acceleration vector produced by the orthogonally-arranged, single-axis sensing elements.

In some examples, the sensing portion 7510 in FIG. 75E may comprise a posture parameter 7547 to direct sensing, received sensed information, etc. regarding posture, which also may comprise sensing of body position, activity, etc. of the patient. Among other uses, in one example implementation, the posture information may support posture parameter 7422 in confidence factor portion 7400 in FIG. 75A and/or in application of confidence factors at 7330 of example method in FIG. 74.

This sensed posture information may be indicative of respiration information in some instances. However, in some example methods and/or devices, via sensing portion 7510, respiration information may be determined without using posture information or body position information. Instead, respiration information may be determined by sensing a change in value of the inclination angle of one or more acceleration sensing elements as the sensing elements move in synchrony with the rotational movements of the chest during breathing, as described throughout examples of the present disclosure. This sensing of rotational movement does not depend on, or involve, determining a posture of the patient.

Nevertheless, as described elsewhere herein, in some examples posture may be considered as one of several parameters when determining respiration information. For instance, sensing an upright posture typically is associated with a wakeful state, such as standing or walking. However, as noted elsewhere, a person could be in an upright sitting position (FIG. 58) and still be in a sleep state (e.g. sleeping a chair). Conversely, sensing a supine or lateral decubitus (i.e. laying on a side) posture typically is considered a sleeping body position or posture. However, a patient might be in such a position without being asleep. Accordingly, posture may be just one parameter used in determining respiration information when in a sleeping body position during a treatment period. Moreover, sensing posture may not be limited to sensing a static posture but extend to sensing simple changes in posture (or body position), which may be indicative of a sleep state/sleep stage at least because certain changes in posture (e.g. from supine to upright) are mostly likely indicative of a wake state. Similarly, more complex or frequent changes in posture and/or body position may be further indicative of a wake state, whereas maintaining a single stable posture for an extended period time may be indicative of a sleep state.

Among other types and/or ways of sensing information, via the accelerometer portion 7530 in FIG. 75E, the accelerometer sensor(s) described herein (and/or other accelerometers) may be employed to sense or obtain ballistocardiograph (BCG) sensing 7535, seismocardiograph (SCG) sensing 7536, and/or accelerocardiograph (ACG) sensing 7538. This sensed information may be used to at least partially determine or confirm respiration information, with such sensed information including heart rate and/or heart rate variability. Among other implementations, such heart rate and/or heart rate variability information may be used as part of implementing heart rate parameter 7424 in confidence factor portion 7400 in FIG. 75A and/or at confidence portion 7330 in FIG. 74.

In some examples, the sensing portion 7510 may comprise an impedance sensing parameter 7550, which may direct sensing of and/or received sensed information regarding transthoracic impedance or other bioimpedance of the patient. In some examples, the impedance sensor 7550 may use a plurality of sensing elements (e.g. electrodes) spaced apart from each other across a portion of the patient’s body, such as electrodes 2820, 2830, 2812, surface of device 2833 (e.g. IPG), etc. in FIGS. 50-51. In some such examples, one of the sensing elements may be mounted on or form part of an external surface (e.g. case) of an implantable pulse generator (IPG) or other implantable sensing monitor, which other sensing elements (e.g. electrodes 2820, 2830 in FIG. 50) may be located at a spaced distance from the sensing element of the IPG or sensing monitor. In at least some such examples, the impedance sensing arrangement integrates all the motion/change of the body (e.g. such as respiratory effort, cardiac motion, etc.) between the sense electrodes (including the case of the IPG when present). Some examples implementations of the impedance measurement circuit will include separate drive and measure electrodes to control for electrode to tissue access impedance at the driving nodes.

With further reference to FIG. 75E, in some examples the sensing portion 7510 may comprise a pressure sensing parameter 7552, which senses respiratory information, such as but not limited to respiratory cyclical information. In some such examples, the respiratory pressure sensor may comprise at least some of substantially the same features and attributes as described in Ni et al., METHOD AND APPARATUS FOR SENSING RESPIRATORY PRESSURE IN AN IMPLANTABLE STIMULATION SYSTEM, published as US 2011/0152706 on Jun. 23, 2011, and which is incorporated herein by reference in its entirety. In some examples, the pressure sensor 7552 may be located in direct or indirect continuity with respiratory organs or tissue supporting the respiratory organs in order to sense respiratory information. In some examples, one of the sensors 2820, 2830, etc. in FIGS. 50-51 may comprise a pressure sensor.

In some examples and as shown in FIG. 75E, sensing portion 7510 may comprise an acoustic sensing parameter 7554 to direct sensing of, and/or receive sensed acoustic information, such as but not limited to cardiac information (including heart sounds), respiratory information, snoring, etc.

In some examples, the sensing portion 7510 of care engine 7500 (FIG. 75E) comprises other parameter 7560 to direct sensing of, and/or receive, track, evaluate, etc. sensed information other than the previously described information sensed via the sensing portion 7510.

In some examples, one sensing modality within sensing portion 7510 may be implemented via another sensing modality within sensing portion 7510.

In some examples, sensing portion 7510 of care engine 7500 may comprise a history parameter 7562 by which a history of sensed physiologic information is maintained, and which may be used via comparison parameter 7564 to compare recent sensed physiologic information with older sensed physiologic information.

As shown in FIG. 75E, in some examples, care engine 7500 may comprise a respiration portion 7580. In at least some examples, in general terms respiration portion 7580 may direct determining respiration information, including sensing of, and/or receiving, tracking, and/or evaluating respiratory morphology, including phase information, general patterns and/or specific fiducials within a respiratory signal. In some examples, the respiration portion 7580 may operate in cooperation with, or as part of sensing portion 7510 in FIG. 75E, which particularly includes (among other things) obtaining or sensing acceleration signal information to sense rotational movement of a patient’s chest. Accordingly, in some examples the respiration portion 7580 comprises a feature extraction portion 7581 to determine respiratory morphology (including phase information) from the sensed acceleration signals regarding rotational movement of the chest wall. In some examples, the feature extraction portion 7581 may be implemented via at least some of the features and attributes as the previously described examples in FIG. 74-75D. With this in mind, as further shown in FIG. 75E, at least some aspects of such respiratory morphology determined, monitored, received, etc. via respiration portion 7580 may comprise inspiration phase morphology (parameter 7582), expiration active phase morphology (parameter 7583), and/or expiratory pause phase morphology (parameter 7584). In some examples, the respective inspiration morphology parameter 7582, expiratory active morphology parameter 7583, and/or expiratory pause morphology parameter 7584 may comprise amplitude, duration, peak (7587), onset (7588), and/or offset (7590) of the respective inspiratory and/or expiratory phases of the patient’s respiratory cycle. In some examples, the detected respiration morphology may comprise transition morphology (7592) such as an inspiration-to-expiration transition and/or an expiration-to-inspiration transition.

In some examples, as further shown in FIG. 75E, the respiration portion 7580 may comprise a confidence parameter 7585 to apply a selectable confidence factor (e.g. level) to different aspects of a filtered, sensed acceleration signal in order to determine the specific respiratory phase information (e.g. inspiration, expiratory active, expiratory pause). In some examples, the confidence parameter 7585 may be implemented, at least in part, via the confidence factor portion 7400 in FIG. 75A and/or as at 7330 in FIG. 74.

In some examples, as further shown in FIG. 75E, the respiration portion 7580 may comprise a default parameter 7586 to use default respiratory phase information in place of a sensed acceleration signal when the sensed signal quality is poor. In some examples, the default parameter 7586 may be implemented, at least in part, via the default respiratory phase portion 7390 in FIG. 74.

In some examples, as further shown in FIG. 75E, the respiration portion 7580 may comprise a slope inversion parameter 7594 to enhance tracking of the phases (e.g. inspiratory, etc.) of the determined respiration information regardless of whether the signal may be inverted relative to a default positive slope, as previously described in various examples of the present disclosure such that the respiration information may be reliably determined regardless of the patient’s rotation in space and/or relative to the gravity vector (in at least some examples). In this regard, it will be noted that the determination of and/or use of the respiration information does not depend on which polarity the signal exhibits, but rather depends, at least partially, on the morphology of the respective phases (e.g. inspiratory, expiratory active, expiratory pause).

In some examples, as shown in FIG. 75E, the respiration portion 7580 may comprise a noise parameter 7596 by which noise is filtered or extracted from the acceleration signal to increase the signal-to-noise ratio for the rotational movement information. In some such examples, the noise parameter 7596 may be implemented via use of a noise model, such as but not limited to the example noise model 7470 in FIG. 75D. In some such examples, the noise parameter 7596 may be implemented in association with at least some aspects of the feature extraction, as previously described in association with at least FIGS. 74 and 75B.

As further shown in FIG. 75E, in some examples the care engine 7500 comprises a SDB parameters portion 7600 to direct sensing of, and/or receive, track, evaluate, etc. parameters particularly associated with sleep disordered breathing (SDB) care. For instance, in some examples, the SDB parameters portion 7600 may comprise a sleep quality portion 7610 to sense and/or track sleep quality of the patient in particular relation to the sleep disordered breathing behavior of the patient. Accordingly, in some examples the sleep quality portion 7610 comprises an arousals parameter 7612 to sense and/or track arousals caused by sleep disordered breathing (SDB) events with the number, frequency, duration, etc. of such arousals being indicative of sleep quality (or lack thereof).

In some examples, the sleep quality portion 7610 comprises a state parameter 7614 to sense and/or track the occurrence of various sleep states (including sleep stages) of a patient during a treatment period or over a longer period of time.

In some examples, the SDB parameters portion 7600 comprises an AHI parameter 7630 to sense and/or track apnea-hypopnea index (AHI) information, which may be indicative of the patient’s sleep quality. In some examples, the AHI information is obtained via a sensing element, such as one or more of the various sensing types, modalities, etc., which may be implemented as described in various examples of the present disclosure.

As further shown in FIG. 75E, in some examples care engine 7500 comprises a stimulation portion 7700 to control stimulation of target tissues, such as but not limited to an upper airway patency nerve (e.g. hypoglossal nerve) and/or a phrenic nerve, to treat sleep disordered breathing (SDB) behavior. In some examples, the stimulation portion 7700 comprises a closed loop parameter 7710 to deliver stimulation therapy in a closed loop manner such that the delivered stimulation is in response to and/or based on sensed patient physiologic information.

In some examples, the closed loop parameter 7710 may be implemented using the sensed information to control the particular timing of the stimulation according to respiratory information, in which the stimulation pulses are triggered by or synchronized with specific portions (e.g. inspiratory phase) of the patient’s respiratory cycle(s). In some such examples and as previously described, this respiratory information may be determined via the sensors, sensing elements, devices, sensing portions (e.g. 7510) as previously described in association with at least FIGS. 1A-75E and FIG. 76A-102.

In some examples in which the sensed physiologic information enables determining at least respiratory phase information, the closed loop parameter 7710 may be implemented to initiate, maintain, pause, adjust, and/or terminate stimulation therapy based on (at least) the determined respiratory phase information (7390).

In some examples, the stimulation is started prior to an onset of the inspiratory phase (7352 in FIG. 74) and the stimulation is stopped exactly at the end of the inspiratory phase or stopped just after the end of the inspiratory phase.

As further shown in FIG. 75E, in some examples the stimulation portion 7700 comprises an open loop parameter 7725 by which stimulation therapy is applied without a feedback loop of sensed physiologic information. In some such examples, in an open loop mode the stimulation therapy is applied during a treatment period without (e.g. independent of) information sensed regarding the patient’s sleep quality, sleep state, respiratory phase, AHI, etc. In some such examples, in an open loop mode the stimulation therapy is applied during a treatment period without (i.e. independent of) particular knowledge of the patient’s respiratory cycle information.

However, in some such examples, some sensory feedback may be utilized to determine, in general, whether the patient should receive stimulation based on a severity of sleep apnea behavior.

As further shown in FIG. 75E, in some examples the stimulation portion 7700 comprises an auto-titration parameter 7720 by which an intensity of stimulation therapy can be automatically titrated (i.e. adjusted) to be more intense (e.g. higher amplitude, greater frequency, and/or greater pulse width) or to be less intense within a treatment period.

In some such examples and as previously described, such auto-titration may be implemented based on sleep quality, which may be obtained via sensed physiologic information, in some examples. It will be understood that such examples may be employed with synchronizing stimulation to sensed respiratory information (i.e. closed loop stimulation) or may be employed without synchronizing stimulation to sensed respiratory information (i.e. open loop stimulation).

In some examples, at least some aspects of the auto-titration parameter 2920 may comprise, and/or may be implemented, via at least some of substantially the same features and attributes as described in Christopherson et al. US 8,938,299, SYSTEM FOR TREATING SLEEP DISORDERED BREATHING, issued Jan. 20, 2015, and which is hereby incorporated by reference in its entirety.

In some examples, as further shown in FIG. 75E, the stimulation portion 7700 of care engine 7500 may comprise an “off period” function 7730 by which a user or clinician may adjust the time that stimulation will remain off and which may be expressed as a percentage of the previous “on period.” In some examples, the “off period” for stimulation coincides with the expiratory active phase 7354 (FIG. 74). However, in some examples, once enabled by the user or clinician, the “off period” (i.e. no stimulation) setting is implemented regardless of detected phases (e.g. 7352, 7354, 7356 in FIG. 74).

In some examples, as further shown in FIG. 74, the stimulation portion 7700 of care engine 7500 may comprise a “maximum stimulation” function 7735 which may be used by a patient or clinician to adjust a maximum time for an “on period” of stimulation for a given stimulation cycle, after which an “off period” takes place. The “on period” may extend for a selectable, predetermined period of time. In some examples, the “on period” for stimulation coincides with the inspiratory phase 7352 (FIG. 74). In some such examples, once enabled by the user or clinician, the “on period” of stimulation is implemented regardless of detected phases (e.g. 7352, 7354, 7356 in FIG. 74).

FIG. 76A-101 are a series of block diagrams and/or flow diagrams schematically representing various example methods. In some examples, the various methods in FIG. 76A-101 may be implemented via at least some of the sensors, sensing element, respiration determination elements, stimulation elements, power/control elements (e.g. pulse generators), devices, user interfaces, instructions, information, engines, functions, actions, and/or methods, as previously described in association with FIG. 1-75E. In some examples, the various methods in FIG. 76A-101 may be implemented via elements other than those previously described in association with FIG. 1-75E.

In some examples, one or more of the example methods in FIG. 76A-101 may be employed together in various combinations. In some examples, one or more of the example methods in FIG. 76A-101 may be employed as part of, and/or together with, the example methods and devices previously described in association with FIG. 1-75E.

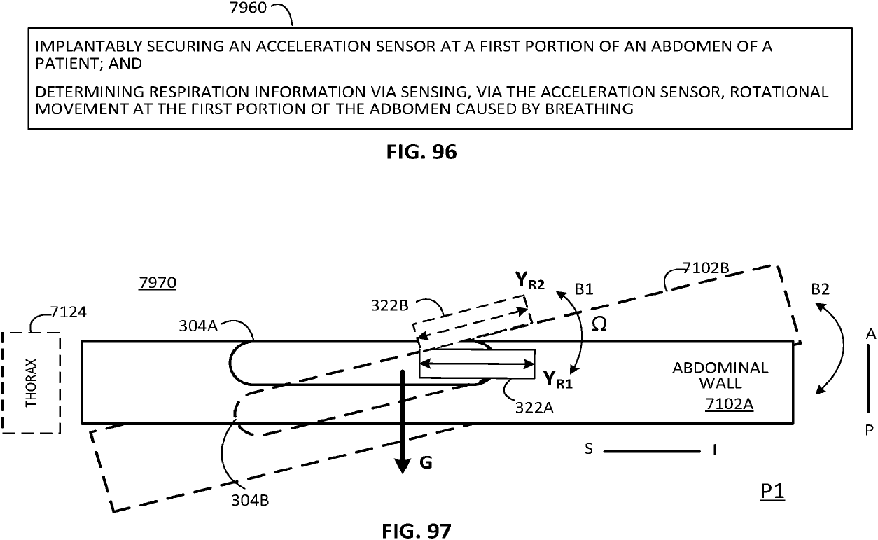

As shown at 7800 in FIG. 76A, some example methods comprise implantably securing an acceleration sensor at a first portion of a respiratory body portion of a patient; and determining respiration information via sensing, via the acceleration sensor, rotational movement at the first portion of the respiratory body portion caused by breathing. In some examples, the respiratory body portion may comprise a chest (e.g. thorax), such as but not limited to, a chest wall, such as described in association with at least FIG. 1A-95. In some examples, the respiratory body portion may comprise an abdomen, such as but not limited to, an abdominal wall, such as described in association with at least FIGS. 96-102 and throughout examples of the present disclosure (e.g. FIG. 1A-95). It will be understood that the respiratory body portion is not necessarily limited to the chest and/or abdomen but in some examples may comprise any other body portion of a patient which exhibits rotational movement caused by breathing and from which sensing of respiration information may be obtained, such as but not limited to, respiration morphology.

As shown at 7820 in FIG. 76B, some example methods comprise implantably securing an acceleration sensor at a first portion of a chest wall of a patient; and determining respiration information via sensing, via the acceleration sensor, rotational movement at the first portion of the chest wall caused by breathing.

As shown at 7830 in FIG. 77, some example methods comprise sensing the rotational movement relative to an earth gravitational field (e.g. gravity vector G).

As shown at 7840 in FIG. 78, some example methods comprise sensing the rotational movement according to at least one of three independent orthogonal axes.

As shown at 7845 in FIG. 79, some example methods comprise combining sensed rotational movement from at least two of the three independent orthogonal axes. Via such combining, the example method may produce composite rotational information (e.g. FIG. 56C) for determining respiration information. In some examples, such combining also may be implemented according to the previously described example methods to perform determination of the composite rotational movement and therefore respiration information at least based on an AC component of a multi-dimensional acceleration vector produced by the n single-axis sensing elements.

As shown at 7850 in FIG. 80, some example methods comprise tracking changes in a value of a first signal, for a first body position during a treatment period, of at least one measurement axis during at least one respiratory period.

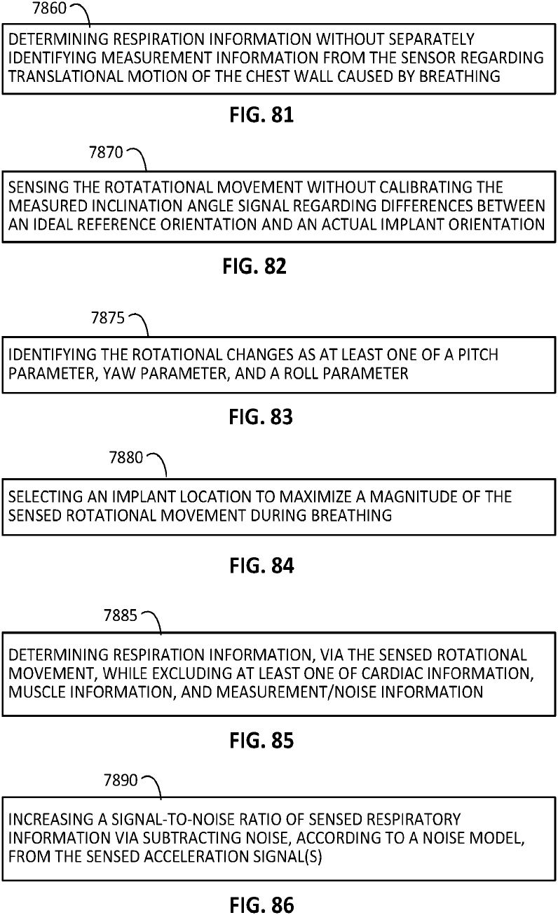

As shown at 7860 in FIG. 81, some example methods comprise determining respiration information without separately identifying measurement information from the sensor regarding translational motion of the chest wall. Via this arrangement, in some example methods/devices, determining the respiration information per acceleration sensing (of the rotational movement at the portion of the chest wall) according to a greatest range of angular orientations (or greatest range of values of the AC signal component) may be performed without directly considering translational motion in determining the respiration information. In some examples, a magnitude of an AC signal component corresponding to rotational movement (of a portion of the chest wall) may be substantially greater than a magnitude of an AC signal component corresponding to translation movement (of the portion of the chest wall). In some examples, at least in this context, the term “substantially greater than” comprises a difference which is 50 percent greater, 100 percent greater, 150 greater, and the like. In some examples, at least in this context, the term “substantially greater than” comprises at least one order of magnitude difference. Accordingly, in at least some such examples, even if some translation movement is sensed, the sensed rotational movement dominates the AC signal component when measuring the inclination angle of the acceleration sensor during rotational movement of the portion of the chest wall during breathing.

As shown at 7870 in FIG. 82, some example methods comprise sensing the rotational movement without calibrating the measured inclination angle regarding differences between an ideal reference orientation and an actual implant orientation.

As shown at 7875 in FIG. 83, some example methods comprise identifying the rotational movement as at least one of a pitch parameter, yaw parameter, and a roll parameter.

As shown at 7880 in FIG. 84, some example methods comprise selecting an implant location to maximize a magnitude of the sensed rotational movement during breathing. In some examples, the implant location, implant orientation, etc. may be selected to ensure a sufficiently high degree of the sensed rotational movement during breathing to accurately and/or reliably determine respiration information (e.g. respiration morphology) even if, and/or when, the sensed rotational movement may not be a maximum obtainable value.

As shown at 7885 in FIG. 85, some example methods comprise determining respiration information, via the sensed rotational movement, while excluding at least one of cardiac noise, muscle noise, and measurement noise. In particular, a sensed acceleration signal is filtered to recover low-frequency respiration signal information while rejecting cardiac noise, measurement noise, and muscle noise. This filtering may employ linear filters, such as low pass filters, high pass filters, band pass filters, and/or may employ non-linear filters, such as median filters and Kalman filters.

As shown at 7890 in FIG. 86, some example methods comprise increasing a signal-to-noise ratio of sensed respiratory information via building a noise model and subtracting the noise model from the sensed acceleration signal. In some examples, the noise model may comprise at least some of substantially the same features and attributes as the noise model previously described at 2470 in FIG. 24E, and which may be used (in some examples) as part of enhancing determination of respiration information in the example method (and/or arrangement) 2300 in FIG. 24A. As previously described, in some examples the noise model may be built via identifying characteristics (e.g. morphology, frequency content, etc.) within sensed acceleration signals of the patient which are caused by various types of activities, positions, environments, etc. which are unrelated to determining respiration information but which may otherwise affect a magnitude and/or direction of the sensed acceleration signal. Once built, the noise model may be subtracted from the sensed acceleration signals, thereby increasing a signal-to-noise ratio of the respiratory features (e.g. morphology) within the sensed acceleration signal(s).

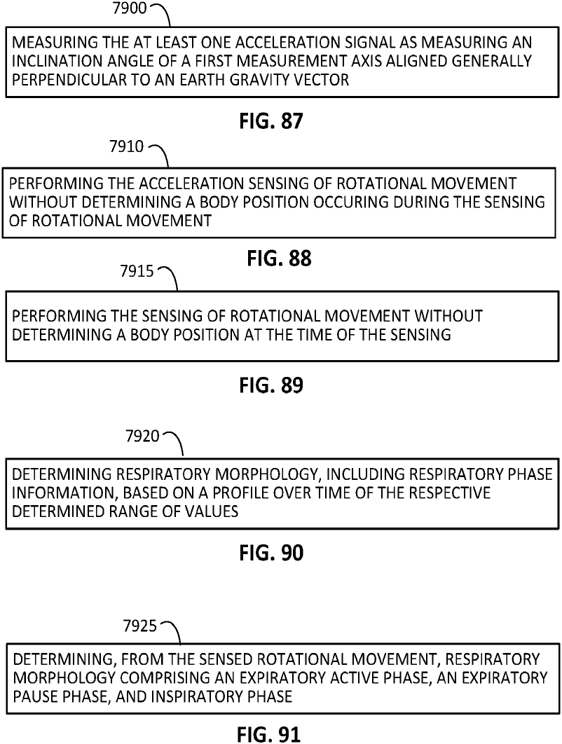

As shown at 7900 in FIG. 87, some example methods comprise measuring the at least one acceleration signal as measuring an inclination angle of a first measurement axis aligned generally perpendicular to an earth gravity vector.

As shown at 7910 in FIG. 88, some example methods comprise performing the acceleration sensing of rotational movement without determining a body position occurring during (e.g. at the time of) the sensing of rotational movement. For example, the sensing may be performed during each of several different sleeping body positions, without determining each different sleeping body position at the time of the sensing.

As shown at 7915 in FIG. 89, some example methods comprise performing the sensing of rotational movement (of a portion of chest wall), during each of several different sleeping body positions, without determining each respective different sleeping body position at the time of sensing of the rotational movement.

As shown at 7920 in FIG. 90, some example methods comprise determining respiratory morphology, including respiratory phase information, based on a profile over time of the respective determined range of values.

As shown at 7925 in FIG. 91, some example methods comprise determining, from the sensed rotational movement, respiratory morphology comprising an inspiratory phase, an expiratory active phase, and an expiratory pause phase.

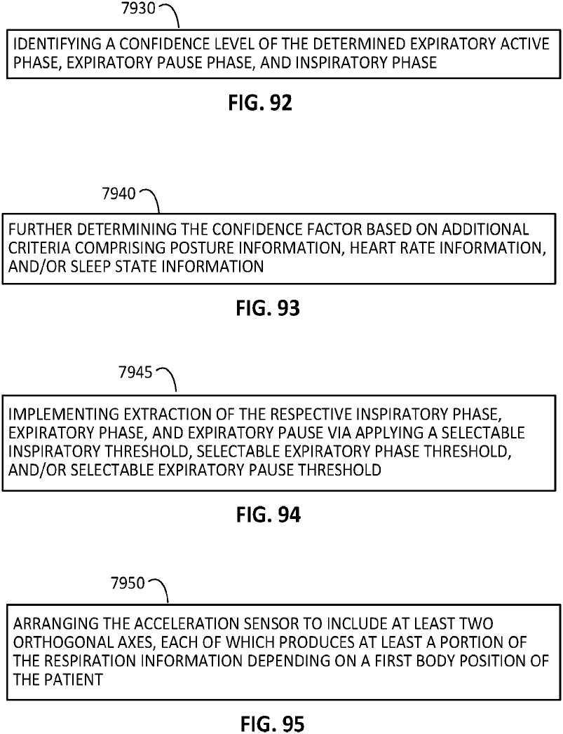

As shown at 7930 in FIG. 92, some example method comprise identifying a confidence factor for the determined inspiratory phase, an expiratory active phase, and an expiratory pause phase.

As shown at 7940 in FIG. 93, some example methods comprise further determining the confidence factor based on additional criteria comprising posture information, heart rate information and/or sleep state information.

As shown at 7945 in FIG. 94, some example methods comprise implementing extraction of the respective inspiratory phase, expiratory active phase, and expiratory pause phase via applying a selectable inspiratory threshold, selectable expiratory active phase threshold, and/or selectable expiratory pause phase threshold.

As shown at 7950 in FIG. 95, some example methods comprises arranging the acceleration sensor to include at least two orthogonal axes, each of which produces at least a portion of the respiration information from the sensed rotational movement depending on a first body position of the patient.

Examples described in association with at least FIGS. 96-102 address determining respiration information via sensing at a respiratory body portion other than the chest, such as but not limited to the abdomen. In some examples, such determination of respiration information may employ at least some of substantially the same features and attributes as previously described in association with FIGS. 1-95, except being applied in the context of the abdomen instead of the chest.

With this in mind, it be further understood that in some examples, sensing in both the chest region and the abdominal region may be performed to determine respiration information and/or to treat sleep disordered breathing. Sensing at the abdomen and sensing at the chest may be performed simultaneously, alternatively, or dependent on the particular physiologic conditions encountered, such as whether central sleep apnea is present, obstructive sleep apnea is present, or whether a multi-type sleep apnea (e.g. both aspects of central and obstructive sleep apnea) is present.

As shown at 7960 in FIG. 96, some example methods comprise implantably securing an acceleration sensor at a first portion of an abdomen of a patient; and determining respiration information via sensing, via the acceleration sensor, rotational movement at the first portion of the abdomen caused by breathing. In some such examples, the abdomen comprises an abdominal wall, which may comprise at least one of an anterior abdominal wall, a lateral abdominal wall, and a posterior abdominal wall, or combinations thereof. In some such examples, in addition to acceleration sensing, at least some of the forms of sensing as previously described in association with at least sensing portion 7510 in FIG. 75E may be used to determine respiration information.

FIG. 97 is a diagram 7970, including a side view, schematically representing an example method and/or example sensor 304A. As shown in FIG. 97, in some examples the sensor 304A may comprise a sensing element 322A, which is arranged to measure an inclination angle (Ω) upon rotational movement of the sensing element 322A caused by breathing. In some examples, the method and/or example sensor 304A in FIG. 97 may comprise at least some of substantially the same features and attributes as the example method and/or example sensor 304A as previously described in association with at least FIGS. 3A-3B, except for being implantably secured at the abdomen to sense rotational movement at the abdomen which is indicative of respiratory information.

The sensor 304A, may be secured on top of, or to, muscle layer(s) of the abdominal wall 7102A, while in some examples, sensor 304A may be secured subcutaneously without being secured on top of the muscle layer(s) of abdominal wall 7102A or without secure to the muscle layer(s) of abdominal wall. In some such examples, the sensor 304A may be secured to non-bony anatomy at the abdomen.

As represented in FIG. 97, upon rotational movement of at least a portion of the abdominal wall 7102A during breathing, the sensing element 322A may rotationally move between a first angular orientation YR1 (shown in solid lines) and a second angular orientation YR2 (shown in dashed lines). In some such examples, the first angular orientation YR1 (shown in solid lines) of sensing element 322A may correspond to a peak expiration of a respiratory cycle (e.g. abdominal wall in collapsed state) and the second angular orientation YR2 (shown in dashed lines) of sensing element 322A may correspond to a peak inspiration of the respiratory cycle (e.g. abdominal wall in expanded state). In a manner similar to that previously described in association with at least FIGS. 3A-3C and FIG. 56A-95, it will be understood that sensing element 322A moves through a range of angular orientations (between at least the first angular orientation YR1 and second angular orientation YR2) and that the respective first and second angular orientations YR1, YR2 generally represent ends of the range and are not fixed positions.

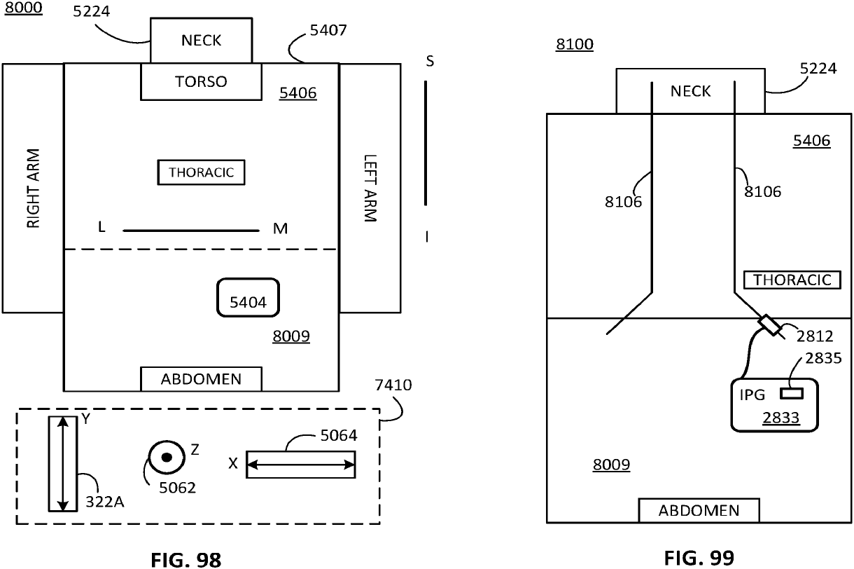

With reference to at least FIG. 97, it will understood that the sensing element 322A moves with at least a portion of the abdominal wall 7102A as depicted in dashed lines. Accordingly, sensing element 322A does not move relative to the abdominal wall 7102A, but rather the sensing element 322A rotationally moves along with (e.g. in synchrony with) the rotational movement of at least the portion of the abdominal wall 7102A (in which the sensor 304A, including sensing element 322A), is implanted) during breathing. As represented in dashed lines 7410 in FIG. 98, the sensor 304A may comprise a sensing element 322A (Y-axis), a sensing element 5062 (Z-axis), and/or a sensing element 5064 (X-axis) having at least some of the features and attributes, as previously described in association with at least FIGS. 3A-3C and FIG. 56A-95.

It will be further understood that the example methods and/or example devices described in FIGS. 96-98 may be implemented, at least in part, according to any one or all of the various examples described in association with FIGS. 1-95, except for the method and/or device in FIGS. 96-98 being applied to sense respiration information via rotational movement of the abdomen caused by breathing instead of via rotational movement of the chest caused by breathing. Accordingly, in some examples, the sensing element 322A comprises an accelerometer, which may comprise a single axis accelerometer in some examples or which may comprise a multiple-axis accelerometer in some examples. Via the accelerometer, the sensing element 322A can determine absolute rotation of sensor 304A (and therefore rotation of the portion of the abdominal wall 7102A) with respect to gravity (e.g. earth gravity vector G), rather than instantaneous changes in rotation. In some examples, element 322A may comprise a single axis accelerometer to measure (at least) a value of, and changes in the value of, the above-noted inclination angle (Ω) associated with movement of at least a portion the abdominal wall 7102A caused by breathing. It will be understood that the use of sensing element 322A may comprise at least some of substantially the same features and attributes of sensing and determining respiration information (such as via sensing rotational movement) as described in association with at least FIGS. 3A-3B and FIG. 56A-95.

In some examples, as shown in FIG. 98, a sensor 5404 (such as in at least FIG. 59-61A) may be implanted in the abdomen 8009 to sense rotational movement at the abdomen to determine respiration information in a manner similar to that previously described in association with at least FIGS. 3A-3C and FIG. 56A-95 (except for the abdomen instead of the chest).

In some examples, the respiration information sensed at the abdomen 8009 may be used in an example method to stimulate a breathing-related nerve, such as an upper-airway-patency-related nerve (e.g. hypoglossal nerve) to treat obstructive sleep apnea, to stimulate a phrenic nerve to treat central sleep apnea, or to stimulate both such nerves to treat multiple-type sleep apnea.

Accordingly, as shown in FIG. 99, in some examples, an acceleration sensor (e.g. 5404 in at least FIG. 59-61A) may be supported by or otherwise associated with an implantable pulse generator (IPG) 2833 (FIGS. 50-51) subcutaneously implanted in the abdomen 8009. The sensor 5404 may be implemented as an accelerometer 2835, as previously described in association with at least FIGS. 50-51. Accordingly, via the accelerometer 2835, example methods and/or devices may determine respiration information. In some example methods and/or devices, a stimulation electrode 2812 is implantable in the abdomen 8009 and supported by the IPG 2833 to be coupled in some manner relative to the phrenic nerve to stimulate the phrenic nerve 8106, such as at an abdominal location. The stimulation electrode 2812 may be a cuff electrode, a paddle electrode, a transvenously deliverable electrode, etc. In some such examples, the stimulation electrode(s) may comprise the sole stimulation elements of the example methods/devices, such that no stimulation electrode is provided to stimulate an upper-airway-patency-related nerve. The example methods and/or devices for such acceleration sensing and/or stimulation in association with FIG. 99 may comprise at least some of substantially the same features and attributes as previously described in association with at least FIGS. 3A-3C and FIG. 56A-98. In some examples in which the acceleration sensor is implanted within the abdominal region to determine respiration information, other sensing modalities may be implanted in the abdominal region as well and/or may be implanted elsewhere, such as in the head-and-neck region and/or in the thoracic region (e.g. pectoral region) as previously described in association with at least FIGS. 3A-3B and FIG. 56A-99. For instance, some example methods and/or devices may employ an abdominally-implanted acceleration sensor (to at least partially determine respiration information) and cardiac-related sensors (e.g. impedance, ECG, etc.) in the thoracic region 5406.

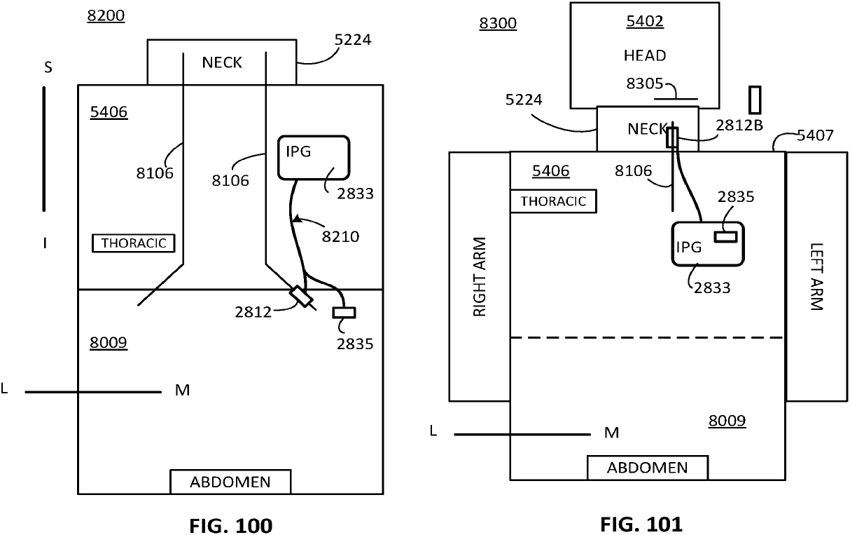

As shown in FIG. 100, in some examples an acceleration sensor (e.g. accelerometer 2835 or single acceleration sensing element) may be implanted in the abdominal region 8009 and a stimulation electrode 2812 may be implanted to be coupled to the phrenic nerve 8106 to stimulate the phrenic nerve. This example may comprise at least some of substantially the same features and attributes as in FIG. 99, except with IPG 2833 being implanted in a thoracic region such as the pectoral region with a single lead 8210 extending from the IPG 2833 to support the accelerometer 2835 and the stimulation electrode 2812.

As shown in the diagram 8300 of FIG. 101, in some examples an acceleration sensor (e.g. accelerometer 2835) may be implanted in a thoracic (e.g. pectoral region) and a stimulation electrode 2812B may be coupled relative to a phrenic nerve 8106 in a head-and-neck region (5402, 5224) or a thoracic region (5406) of the patient’s body.

As shown in the diagram 8310 of FIG. 102, in some example methods and/or example devices, both a pulse generator 2833 and associated stimulation electrode 2812 for stimulating the phrenic nerve 8106 may be located in a head-and-neck region, such as when the pulse generator 2833 and stimulation electrode 2812 together take the form of an example microstimulator 8310. In some such examples, one stimulation electrode 2812A of the microstimulator 8310 may be implanted in a head-and-neck region to stimulate an upper-airway-patency-related nerve 2805 (e.g. hypoglossal nerve) and another separate stimulation electrode 2812B of the microstimulator 8310 may be implanted in the head-and-neck region to stimulate the phrenic nerve 8106. In some such examples, both nerves may be stimulated (although not necessarily simultaneously) in a method of treating multi-type sleep apnea. In some examples, the microstimulator 8310 in FIG. 102 may comprise at least some of substantially the same features and attributes as the microstimulator 2819B previously described in association with at least FIG. 51.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method comprising:
sensing physiologic information via a sensor; and
identifying, via the sensed physiologic information, a disease burden indicator.

2-136. (canceled)
